SEQUENCE LISTING (7 sequences)

SEQ ID NO: 1 - DNA, 487 bp, synthetic construct
  source 1..487: mol_type = other DNA
gacagttactctcactcgtgttcttctctaacaacagagttttacgatccgaagaccttcttcactcacgcggcgttgctccatcagacttgcgtccattgtggaagattccctactgctgcctcccgtaggagtttgggccgtgtctcagtcccaatgtggccgatcaacctctcagttcggctacgtatcatcaccttggtaggccgttaccccaccaactagttaatacgccgcgggtccatctaaaagcgatagcagaaccatctttcatctaaggatcatgcgatccttagagatatacggtattagcacctgtttccaagtgttatccccttcttttaggcaggttacccacgtgttactcacccgtccgccactcaacttcttacggtgaatgcaagcattcggtgtaagaaagtttcgttcgacttgcatgtattaggcacgccgccagcgttcgtcctgagccaggntcaaactctan

SEQ ID NO: 2 - DNA, 1575 bp, synthetic construct
  source 1..1575: mol_type = other DNA
tagaaaggaggtgatccagccgcaggttctcctacggctaccttgttacgacttcaccccaatcatctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcgtccccaccttcctccggtttgtcaccggcagtctcgccagagtgcccaacttaatgctggcaactgacaacaagggttgcgctcgttgcgggacttaacccaacatctcacgacacgagctgacgacagccatgcaccacctgtcactttgtccccgaagggaaagcctaatctcttaggtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggaatgcttattgcgttagctgcggcactgaagggcggaaaccctccaacacctagcattcatcgtttacggcgtggactaccagggtatctaatcctgtttgctacccacgctttcgaacctcagcgtcagttacagaccagagagccgctttcgccactggtgttcttccatatatctacgcatttcaccgctacacatggagttccactctcctcttctgcactcaagtcttccagtttccaatgcactactccggttaagccgaaggctttcacatcagacttaaaagaccgcctgcgttccctttacgcccaataaatccggacaacgcttgccacctacgtattaccgcggctgctggcacgtagttagccgtgacttgctggttagataccgtcatcgaatgaacagttactctcactcgtgttcttctctaacaacagagttttacgatccgaagaccttcttcactcacgcggcgttgctccatcagacttgcgtccattgtggaagattccctactgctgcctcccgtaggagtttgggccgtgtctcagtcccaatgtggccgatcaacctctcagttcggctacgtatcatcaccttggtaggccgttaccccaccaactagttaatacgccgcgggtccatctaaaagcgatagcagaaccatctttcatctaaggatcatgcgatccttagagatatacggtattagcacctgtttccaagtgttatccccttcttttaggcaggttacccacgtgttactcacccgtccgccactcaacttcttacggtgaatgcaagcattcggtgtaagaaagtttcgttcgacttgcatgtattaggcacgccgccagcgttcgtcctgagccaggatcaaactctcattttaaagtttgtga

SEQ ID NO: 3 - DNA, 306 bp, synthetic construct
  source 1..306: mol_type = other DNA
accttatgacaattctcatcactcgtgttcttctctaacaacagagttttacgatccgaagaccttcttcactcacgcggcgttgctccatcagacttgcgtccattgtggaagattccctactgctgcctcccgtaggagtttgggccgtgtctcagtcccaatgtggccgatcaacctctcaggtcggctacgtatcatcaccttggtaggccgttaccccaccaactagttaatacgccgcggggacctctaaaagcgatagcagaaacctctttcttctaaggaacccgcgatcctcaaaag

SEQ ID NO: 4 - DNA, 10 bp, synthetic construct
  source 1..10: mol_type = other DNA
acgcaggcac

SEQ ID NO: 5 - DNA, 10 bp, synthetic construct
  source 1..10: mol_type = other DNA
aacgcgcaac

SEQ ID NO: 6 - DNA, 10 bp, synthetic construct
  source 1..10: mol_type = other DNA
ccgcagccaa

SEQ ID NO: 7 - DNA, 10 bp, synthetic construct
  source 1..10: mol_type = other DNA
aacgggcaga

(19) United States
(12) Patent Application Publication
(10) Pub. No.: US 2026/0250626 A1
(43) Pub. Date: Aug. 27, 2026

(54) LACTOBACILLUS SALIVARIUS STRAINS AND USES THEREOF

(71) Applicant: CHEMO RESEARCH, S.L., Madrid (ES)

(72) Inventors: José Miguel RIZO MARTÍNEZ, Madrid (ES); Pedro Antonio REGIDOR BRANDAU, Madrid (ES); Rocío GUTIÉRREZ HIDALGO, Madrid (ES); Loreto MUÑOZ OTAEGUI, Madrid (ES); Juan Miguel RODRÍGUEZ GÓMEZ, Madrid (ES); Belén ORGAZ MARTÍN, Madrid (ES); Leónides FERNÁNDEZ ÁLVAREZ, Madrid (ES)

(21) Appl. No.: 18/993,953

(22) PCT Filed: Jul. 14, 2023
(86) PCT No.: PCT/EP2023/069670

§ 371 Date: Jan. 13, 2025

(30) Foreign Application Priority Data
Jul. 15, 2022 (EP) 22382679.3

(51) Int. Cl. C12N 1/205; A23C 9/123; A61K 8/99; A61K 35/00; A61K 35/747; A61P 15/08; A61P 19/06; A61P 31/14; A61Q 19/00; C12R 1/225

(52) U.S. Cl. CPC C12N 1/205; A23C 9/1234; A61K 8/99; A61K 35/747; A61P 15/08; A61P 19/06; A61P 31/14; A61Q 19/00; A23V 2400/181; A61K 2035/115; C12R 2001/225

(57) ABSTRACT

The present invention provides new isolated strains of the microorganism Ligilactobacillus salivarius, compositions comprising it and uses thereof in the prevention and/or treatment of infertility and infection diseases as well as non-therapeutic applications.

### TECHNICAL FIELD OF THE INVENTION

The present invention provides new isolated strains of the species Ligilactobacillus salivarius, compositions comprising it and uses thereof in the prevention and/or treatment of inflammation, infertility and infection diseases.

### BACKGROUND OF THE INVENTION

It is becoming more and more apparent that human microbiota complex interactions with its host have several and important associations within the host health, in particular with the human reproductive system.

Recent work has shed light in the relation between microbiota and the reproductive human system, indicating the existence of a physiologic microbiota associated with the mucosal surfaces both the female and male reproductive systems. Furthermore, vaginal disbiosis due to microbiota imbalance has been associated with intraamniotic infection, premature birth, spontaneous abortion, infertility, and neonatal sepsis, one of the major causes of neonatal mortality and morbidity.

The newly acquired knowledge has help to explain some of previously unknown cause recurrent abortions, preterm delivery and neonatal sepsis as well as provided with new tools in the field of fertility and assisted reproduction. One field that is actively being researched is the search for adequate strains of bacteria, which can colonize the female reproductive system leading to an improvement of the necessary conditions for reproduction. The vaginal microbiota of healthy women is usually characterized by the dominance of the genus Lactobacillus species and, particularly, of those belonging to the species L. crispatus, L. gasseri, L. iners and L. jensenii, which may account for even higher than 90% of the vaginal microbiota. Several factors may lead to an alteration of the composition of the vaginal microbiota. Any change involving a significant decrease of the lactobacilli population and an increase in the concentration of strict anaerobes is considered as a risk for developing bacterial vaginosis (BV). In addition, any deviation from a low diversity and Lactobacillus-dominated vaginal microbiome is frequently associated with negative gynecological and obstetric outcomes, including infertility. Similarly, bacteriome studies focused on endometrial samples have shown that a low percentage of Lactobacillus sequences is a common signature among infertile women while the contrary is associated with a higher implantation success. Consequently, there is a growing awareness of the importance of the microbiota of the female genital tract for reproductive success.

Therefore, there is a current need for alternative tools to provide improvements and help in the treatment of conditions afflicting the female genital tract and causing reproduction problems.

### SUMMARY OF THE INVENTION

The inventors of the present invention have found new strains of Ligilactobacillus salivarius, which are shown to have beneficial effects in the improvement of fertility as well as in the reduction of repeated spontaneous abortions.

A first aspect of the present invention is an isolated strain of Ligilactobacillus salivarius capable of metabolizing circulating estrogens wherein the strain is the strain deposited at the “Colección Española de Cultivos Tipo (CECT)” under the accession number CECT 30632 also “MP98” or “ES 27” strain) or a variant thereof, or the isolated strain of Ligilactobacillus salivarius deposited at the “Colección Española de Cultivos Tipo (CECT)” under the accession number CECT 30641 also “ES 43” strain) or a variant thereof.

A following aspect is a bacterial culture comprising at least one of the strains according to the invention.

Another aspect relates to a pharmaceutical composition comprising a therapeutic effective dose of the isolated strain according to the invention or the bacterial culture according to the invention and an acceptable adjuvant.

One more aspect relates to the isolated strain according to the invention, the bacterial culture according to the invention or a pharmaceutical composition comprising a therapeutic effective dose of the isolated strain according to the invention or the bacterial culture according to the invention and an acceptable adjuvant for use in the prevention and/or treatment of a disease associated with high levels of estrogens.

Yet another aspect relates to the isolated strain according to the invention, the bacterial culture according to the invention a pharmaceutical composition comprising a therapeutic effective dose of the isolated strain according to the invention or the bacterial culture according to invention and an acceptable adjuvant for use in the prevention and/or treatment of an infectious disease or an inflammatory disease.

One more aspect of the present invention relates to the isolated strain according to the invention, the bacterial culture according to the invention or a pharmaceutical composition comprising a therapeutic effective dose of the isolated strain according to the invention or the bacterial culture according to invention and an acceptable adjuvant for use in the prevention and/or treatment of a disease or syndrome selected from hyperuricemia, high cholesterol, high triglycerides and oxidative stress.

Yet another aspect relates to the isolated strain according to the invention, the bacterial culture according to the invention or a pharmaceutical composition comprising a therapeutic effective dose of the isolated strain according to the invention or the bacterial culture according to invention and an acceptable adjuvant for use in the prevention and/or treatment of primary or secondary infertility.

Another aspect of the present invention relates to the isolated strain according to the invention, the bacterial culture according to the invention or a pharmaceutical composition comprising a therapeutic effective dose of the isolated strain according to the invention or the bacterial culture according to invention and an acceptable adjuvant for use in increasing the rate of reproductive success in a patient who has suffered from recurrent pregnancy loss.

A further aspect relates to the isolated strain according to the invention, the bacterial culture according to the invention, or a pharmaceutical composition comprising a therapeutic effective dose of the isolated strain according to the invention or the bacterial culture according to invention and an acceptable adjuvant for use in the prevention of miscarriage in the first trimester of pregnancy.

In another aspect, the invention relates to the use of the isolated strain according to the invention, or the bacterial culture according to the invention as a probiotic.

A further aspect relates to a food product comprising the isolated strain according to the invention or the bacterial culture according to the invention and an appropriate amount of an edible ingredient.

Additionally, the invention relates to a nutraceutical comprising the isolated strain according to the invention or the bacterial culture according to the invention.

The invention, also relates to several non-therapeutic methods. In particular, the invention relates to a non-therapeutic method for the regulation of the epithelial mucosa pH in a subject comprising the administration of the isolated strain according to the invention, the bacterial culture according to the invention, the food product according to the invention or the nutraceutical according to the invention to the subject. In another aspect, the invention relates to a non-therapeutic method for the regulation of the microbiota cervicovaginal in a subject comprising the administration of the isolated strain according to the invention, the bacterial culture according to the invention, the food product according to the invention or the nutraceutical according to the invention to the subject.

In another aspect, the invention relates to a non-therapeutic method for promoting an increase in the content of vaginal Lactobacillus species in a subject comprising the administration of the isolated strain according to the invention, the bacterial culture according to the invention, the food product according to the invention or the nutraceutical according to the invention to the subject.

In another aspect, the invention relates to a non-therapeutic method for promoting an increase in soluble immune factors in the internal mucosal of a subject comprising the administration of the isolated strain according to the invention, the bacterial culture according to the invention, the food product according to the invention or the nutraceutical according to the invention to the subject.

In another aspect, the invention relates to a non-therapeutic method for improving the mental well-being of a subject comprising the administration of the isolated strain according to the invention, the bacterial culture according to the invention, edible product according to the invention or the nutraceutical according to the invention to the subject.

In another aspect, the invention relates to a non-therapeutic method for regulating the circulating estrogen levels in a subject comprising the administration of the isolated strain according to the invention, the bacterial culture according to the invention, edible product according to the invention or the nutraceutical according to the invention to the subject.

In another aspect, the invention relates to a non-therapeutic method for reducing the levels of circulating uric acid in a subject comprising the administration of the isolated strain according to the invention, the bacterial culture according to the invention, edible product according to a the invention or the nutraceutical according to the invention to the subject.

In another aspect, the invention relates to a non-therapeutic method for increasing the performance of activities of daily living in a subject comprising the administration of the isolated strain according to the invention, the bacterial culture according to the invention, edible product according to the invention or the nutraceutical according to the invention to the subject.

In another aspect, the invention relates to a non-therapeutic method for increasing the nutritional state of a subject comprising the administration of the isolated strain according to the invention, the bacterial culture according to the invention, edible product according to the invention or the nutraceutical according to the invention to the subject.

In another aspect, the invention relates to a non-therapeutic method for regulating the circulating levels of total cholesterol of a subject comprising the administration of the isolated strain according to the invention, the bacterial culture according to the invention, edible product according to the invention or the nutraceutical according to the invention to the subject.

In another aspect, the invention relates to a non-therapeutic method for regulating the circulating levels of triglycerides of a subject comprising the administration of the isolated strain according to the invention, the bacterial culture according to the invention, edible product according to the invention or the nutraceutical according to the invention to the subject.

The invention also relates to a medical device comprising an effective amount of the isolated strain according to the invention or the bacterial culture according to the invention as an active ingredient.

The invention also relates to a cosmetic composition having an effect of reducing vaginal discharge comprising the isolated strain according to the invention or the bacterial culture according to the invention.

### BRIEF DESCRIPTION OF THE FIGURES

FIG. 1. Dominant lactobacilli species (when lactobacilli could be isolated) in CVL samples of healthy fertile women (C), women with a history of repetitive abortion (RA) and women with infertility of unknown origin (INF).

FIG. 2. Comparison of selected baseline demographic characteristics (age, weight, and height) and vaginal parameters (pH, Nugent score, TGF-β 1, TGF-β 2, and VEGF concentrations, and viable Lactobacillus counts in CVL samples) of women with repetitive abortion (RA) and women with infertility of unknown origin (INF) at recruitment. For each boxplot, the line and the cross within the box represent the median and mean, respectively. The bottom and top boundaries of each box indicate the first and third quartiles (the 25th and 75th percentiles), respectively. The whiskers represent the lowest and highest values within the 1.5 interquartile range (IQR) and the dots outside the rectangles are suspected outliers (>1.5×IQR). One-way ANOVA tests were used to compare both groups.

FIG. 3. Changes in vaginal parameters (pH, Nugent scorre, TGF-β 1, TGF-β 2, and VEGF concentrations, viable Lactobacillus counts and L. salivarius copies in CVL samples) in women with a history of reproductive failure, because either of recurrent miscarriage (RA group) or infertility (INF groups), after the probiotic intervention with L. salivarius MP98 according to their outcome (pregnancy versus no pregnancy).

FIG. 4. Box-plots showing MRS bacterial concentration (log 10 CFU/container) (A) and pH (B) values of the dairy products over time. The boxes represent the values of the interquartile ranges, with the median represented as a line. Outliers are represented as dots.

FIG. 5. Estrone metabolization measured by HPLC/MS in the different strains selected.

FIG. 6. Estriol metabolization measured by HPLC/MS in the different strains selected.

FIG. 7. 17B-estradiol metabolization measured by HPLC/MS in the different strains selected

FIG. 8. Global assessment of estrone metabolization rate.

FIG. 9. Global assessment of estriol metabolization rate.

FIG. 10. Global assessment of 17β-estradiol metabolization rate FIG. 11. Global assessment of estrone conjugation rate.

FIG. 12. Global assessment of estriol conjugation rate.

FIG. 13. Global assessment of 17β-estradiol conjugation rate.

FIG. 14. Metabolization of E1G (A) and E2G (B) by adding exogenous E1G and E2G to the culture by ES27 and ES43 for 48 h at 37° C.

### DETAILED DESCRIPTION OF THE INVENTION

The authors of the present invention have isolated strains of Ligilactobacillus salivarius, which have been deposited at the “Colección Española de Cultivos Tipo (CECT)” under accession numbers CECT 30632 and CECT 30641. The strains have estrogen metabolizing capabilities and co-aggregative and antimicrobial activity against a wide spectrum of vaginal pathogens while at the same time persevering the growth of vaginal native lactobacilli. The strains can therefore modulate the vaginal ecosystem and lead to better fertility outcomes.

### Isolated Strain and Bacterial Culture

A first aspect of the present invention refers to an isolated strain of Ligilactobacillus salivarius, from here onwards the strain of the invention, capable of metabolizing circulating estrogens wherein said strain is the strain deposited at the “Colección Española de Cultivos Tipo (CECT)” under the accession number CECT 30632 or a variant thereof, or the strain deposited at the “Colección Española de Cultivos Tipo (CECT)” under the accession number CECT 30641 or a variant thereof.

In a preferred embodiment, the isolated strain of Ligilactobacillus salivarius capable of metabolizing circulating estrogens is the strain deposited at the “Colección Española de Cultivos Tipo (CECT)” under the accession number CECT 30632 (also “MP 98” or “ES 27” strain or a variant thereof. In another preferred embodiment, the isolated strain of Ligilactobacillus salivarius capable of metabolizing circulating estrogens is the strain deposited at the “Colección Española de Cultivos Tipo (CECT)” under the accession number CECT 30641 (also “ES 43”) or a variant thereof.

As used herein the term “isolated strain” refers to a bacterial strain that has been removed from its natural milieu. The term “isolated” does not necessarily reflect the extent to which the microbe has been purified. In contrast, the phrase “substantially pure culture” refers to a microbial culture that contains substantially no other microbes other than the desired strain or strains, and substantially free of other contaminants, which can include microbial contaminants as well as unwanted chemical contaminants.

Methods for the isolation of are widely known in the art. One such method consists in spreading samples obtained from the tissue of interest, e.g. a sample of the vaginal mucosa, onto selective agar plates followed by incubation in aerobic or anaerobic conditions as to isolate bacteria. Follow isolation, bacteria can be identified by 16S rDNA sequencing (Kullen et al., J. Appl. Microbiol. 2000; 89:511-518).

The term “Ligilactobacillus salivarius” or “Lactobacillus salivarius” as used herein refers to an isolated strain of the species Ligilactobacillus salivarius having identifying characteristics that render the strain suitable for use with the present invention. Exemplary non-limiting identifying characteristics can include circulating estrogen metabolizing activity, antimicrobial activity against vaginal and/or cervical pathogens, ability to co-aggregate with vaginal and cervical pathogens, adhesion to vaginal epithelial cells and mucus, high α-amylase activity, and/or high production of lactic acid production. A particular strain suitable for use with the present invention can exhibit one or more identifying characteristics.

The strain of the invention is capable of metabolizing circulating estrogens, such as estrone, estriol and 17-β-estradiol. The term “estrogen” as used herein refers to a category of sex hormone responsible for the development and regulation of the female reproductive system and secondary sex characteristics. The term is meant to encapsulate all three major endogenous estrogens that have estrogenic hormonal activity: estrone, estradiol, and estriol. In a particular embodiment of the strain of the invention, the estrogens are selected from the group consisting of estriol estrona, 17β-estradiol, 2-hidroxi-3-metileter, 2-metox-estradiol, 4-metoxi-17β-estradiol, 4-metoxiestrona, 16α-hidroxiestrona, 2-hidroxiestrona, 4-hidroxiestrona and 17-epiestriol. In a more particular embodiment, the estrogens are selected from the group consisting of estrone, estriol, 17 β-estradiol or any combination thereof. In a more preferred embodiment, the estrogens are selected form the group consisting of estrone and estriol, estrone and 17 β-estradiol, estriol and 17 β-estradiol, and estrone, estriol and 17 β-estradiol. “Capable of metabolizing circulating estrogens”, as used herein relates to the ablity to degradate or transform circulating estrogens.

The term “circulating estrogens” refers to estrogens that can be detected in biological fluids such as the blood and urine.

Estrogen levels can be measured, directly or indirectly, in blood serum or plasma utilizing a variety of assays such as an enzyme linked immunosorbent assay (ELISA) or a Western blot analysis or by HPLC/MS).

The term “vaginal and/or cervical pathogen” refers to a microorganism, mainly a fungus or a bacterium, capable of colonizing the mucosa of the vagina and/or the cervix, potentially capable of producing a disease, such as vaginitis, vaginosis, trichomoniasis, candidiasis, or lower urinary tract infections. The term “vaginal pathogen” includes both microorganisms of external origin and microorganisms that, although they may be present in a healthy vaginal mucosa, can give rise to disease when there is an imbalance in the microbiota of the vagina, including a decrease in certain microorganisms that have a beneficial or protective effect, and/or an increase in the potentially pathogenic microorganism. Illustrative, non-limiting examples of vaginal pathogens include Candida albicans, Candida glabrata, Candida tropicalis, Gardnerella vaginalis, Streptococcus agalactiae, Ureaplasma urealyticum, Trichomonas vaginalis, Neisseria gonorrhoeae, Chlamydia trachomatis, Mycoplasma hominis and Prevotella spp. In a particular embodiment, the vaginal pathogen is selected from the group consisting of Gardnerella vaginalis (G. vaginalis MP14, G. vaginalis MP17, G. vaginalis MP20, G. vaginalis MP24, G. vaginalis MP29), Streptococcus agalactias (S. agalactiae MP07, S. agalactiae MP12, S. agalactiae MP46), Candida albicans (C. albicans MP09, C. albicans MP18, C. albicans MP31), Candida glabrata (C. glabrata MP33, C. glabrata MP37), Candida parapsilosis (C. parapsilosis MP36, C. parapsilosis MP48), and Ureaplasma urealyticum (U. urealyticum MP39, U. urealyticum MP57).

The term “antimicrobial activity” as used herein refers to the ability to kill or inhibit the growth of a microorganism, in this case the vaginal pathogen. Antimicrobial activity includes bactericidal activity (ability to kill bacteria), bacteriostatic activity (ability to inhibit the growth of bacteria), fungicidal activity (ability to kill fungi), and fungistatic activity (ability to inhibit the growth of fungi). In a particular embodiment, the strain of the invention has antimicrobial activity against a microorganism selected from the group consisting of: Streptococcus agalactiae, Gardnerella vaginalis, Ureaplasma urealyticum, Escherichia coli, Escherichia coli OBR 01, Klebsiella pneumoniae, Klebsiella pneumoniae K12 4, Klebsiella pneumoniae Kp5, Enterococcus faecalis, Enterococcus faecalis OEA1, Enterococcus faecium, Enterococcus faecium EIPO, Candida albicans, Candida glabrata, Candida parapsilosis and any combination thereof. The antimicrobial activity can be determined by several methods known in the literature such as the overlay method for both bacteria and fungi (Magnusson et al., Appl. Environ. Microbiol. 2001, 67, 1-5, Martín et al., Int. J. Food Microbiol. 2006, 112, 35-43) used in the examples of the present description. In a particular embodiment of the strain of the invention or the variant thereof, the isolated strain of the invention has antimicrobial activity against a vaginal and/or cervical pathogen selected from G. vaginalis, Streptococcus agalactiae, Candida albicans, Candida glabrata, Candida parapsilosis, Ureaplasma urealyticum, Escherichia coli, Escherichia coli OBR-01, Klebsiella pneumoniae, Klebsiella pneumoniae K12-4, Klebsiella pneumoniae Kp5, Enterococcus faecalis, Enterococcus faecalis OEA1, Enterococcus faecium, Enterococcus faecium EIPO and any combination thereof.

The term “ability to co-aggregate” refers to the ability of the isolated strain of the invention to adhere to other microorganisms of different species and can be assessed as described in Boris et al. (Boris et al., Infect. Immun. 1998, 66, 1985-1989). In a more particular embodiment, the isolated strain of the invention has the ability to co-aggregate with a vaginal and/or cervical pathogen selected from G. vaginalis, Streptococcus agalactiae, Candida albicans, Candida glabrata, Candida parapsilosis, Ureaplasma urealyticum, Escherichia coli, Escherichia coli OBR-01, Klebsiella pneumoniae, Klebsiella pneumoniae K12-4, Klebsiella pneumoniae Kp5, Enterococcus faecalis, Enterococcus faecalis OEA1, Enterococcus faecium, Enterococcus faecium EIPO and any combination thereof.

The term “adhesion” refers to the ability of the isolated strain of the invention to bind to host tissues, in this case, vaginal epithelial cells and mucus. In a particular embodiment, the isolated strain of the invention has a high adhesion to vaginal epithelial cells and mucus. The level of adhesion to vaginal epithelial cells and mucus can be determine by methods previously described (Boris et al., Infect. Immun. 1998, 66, 1985-1989; Cohen et al., Methods Enzymol. 1995, 253, 309-314). It is considered that the adhesion to vaginal epithelial cells and mucus of the isolated strain of the invention is high when it is at least 90% of the adhesion of that of Lactobacillus salivarius CECT 9145 strain when it is determined under the same experimental conditions, more particularly, at least 91%, at least one 92%, at least 93%, at least 94%, at least 95%, at least 96%, at least 97%, at least 98%, at least 99%, 100% or higher.

The term “α-amylase activity”, as used here, refers to the enzymatic activity identified by number EC 3.2.1.1 capable of hydrolyzing glycosidic bonds along any point of the carbohydrate chain, breaking them down to generate maltotriose and maltose from amylose and maltose, glucose and dextrin from amylopectin. The α-amylase activity of a bacterium is considered high when it is greater than 0.50 U/mL when it is determined by the method of Narita et al. (2006), Appl. Environ. Microbiol. 72:269-275. In a particular embodiment of the isolated strain of the invention, the isolates strain has a high α-amylase activity of at least 0.50 U/mL at 16 hours.

The term “lactic acid production”, as used herein, refers to the ability of the strain to produce lactic acid, whose official nomenclature is 2-hydroxypropanoic acid or α-hydroxypropanoic acid, in either of its two L or D isomers. Lactic acid production can be determined by any known test, for example, by the method described by Martin et al. (Martín et al. 2006. Int. J. Food Microbiol. 112:35-43), which is based on the quantification of both enantiomers of lactic acid by means of an enzymatic kit in the supernatant of a culture in MRS (Man, Rogosa and Sharpe) broth (see Example section for details). Lactic acid production of a bacterium is considered high when it is greater than 8 mg/ml in culture supernatants after incubation under optimal growth conditions. In a particular embodiment of the isolates strain of the invention, the isolates strain has a lactic acid production of at least 8 mg/ml. In a preferred embodiment, the lactic acid is L-lactic acid, which corresponds to the compound having the CAS number 79-33-4.

In a particular embodiment of the isolates strain of the invention, the isolated strain is a variant of the isolated strain of the invention, which maintains at least one of the characteristics selected from a group consisting of: circulating estrogen metabolizing activity, antimicrobial activity, ability to co-aggregate, adhesion, high α-amylase activity, lactic acid production, or any combination thereof. The term “maintains”, as used herein, refers to the mutant showing an activity of 100% or greater or of at least 99%, at least 98%, at least 97%, at least 96%, at least 95%, at least 94%, at least 93%, at least 92%, at least 91%, at least 90%, at least 85%, at least 80%, at least 75%, at least 70%, at least 60% or at least 50% of the activity of the isolated strain of the invention from which it is derived, when this property is measured by adequate and quantitative assays.

In a particular embodiment, the variant of the isolated strain of the invention maintains the antimicrobial activity against vaginal and/or cervical pathogens of the isolated strain of the invention. In a more particular embodiment, the variant of the isolates strain of the invention maintains the antimicrobial activity against a vaginal and/or cervical pathogen selected from G. vaginalis, Streptococcus agalactiae, Candida albicans, Candida glabrata, Candida parapsilosis, Ureaplasma urealyticum, Escherichia coli, Escherichia coli OBR-01, Klebsiella pneumoniae, Klebsiella pneumoniae K12-4, Klebsiella pneumoniae Kp5, Enterococcus faecalis, Enterococcus faecalis OEA1, Enterococcus faecium, Enterococcus faecium EIPO and any combination thereof. In a particular embodiment the variant of the isolated strain of the invention maintains 100% or more, or at least 99%, at least 98%, at least 97%, at least 96%, at least 95%, at least 94%, at least 93%, at least 92%, at least one 91%, at least 90%, at least 85%, at least 80%, at least 75%, at least 70%, at least 60% or at least 50% of the antimicrobial activity of the isolated strain of the invention on the vaginal and/or cervical pathogen, preferably a pathogen selected from the group consisting of G. vaginalis, Streptococcus agalactiae, Candida albicans, Candida glabrata, Candida parapsilosis, Ureaplasma urealyticum, Escherichia coli, Escherichia coli OBR-01, Klebsiella pneumoniae, Klebsiella pneumoniae K12-4, Klebsiella pneumoniae Kp5, Enterococcus faecalis, Enterococcus faecalis OEA1, Enterococcus faecium, Enterococcus faecium EIPO and any combination thereof, when said activity is determined by the overlay test mentioned herein.

In a particular embodiment, the variant of the isolated strain of the invention maintains the ability to co-aggregate with vaginal and/or cervical pathogens of the isolated strain of the invention. In a more particular embodiment, the variant maintains the ability of co-aggregating with a vaginal and/or cervical pathogen selected from the group consisting of G. vaginalis, Streptococcus agalactiae, Candida albicans, Candida glabrata, Candida parapsilosis, Ureaplasma urealyticum, Escherichia coli, Escherichia coli OBR-01, Klebsiella pneumoniae, Klebsiella pneumoniae K12-4, Klebsiella pneumoniae Kp5, Enterococcus faecalis, Enterococcus faecalis OEA1, Enterococcus faecium, Enterococcus faecium EIPO and any combination thereof. In a particular embodiment, the variant maintains the ability to co-aggregate with vaginal and/or cervical pathogens of the isolates strain of the invention when said activity is determined by the co-aggregation assay described by Boris et al. (Infect. Immun. 1998, 66, 1985-1989). In a particular embodiment, the variant maintains 100% or more, or at least 99%, at least 98%, at least 97%, at least 96%, at least 95%, at least 94%, at least 93%, at least 92%, at least 91%, at least 90%, at least 85%, at least 80%, at least 75%, at least 70%, at least less 60% or at least 50% of the co-aggregating capacity of the isolated strain of the invention on the vaginal and/or cervical pathogen, preferably a pathogen selected from the group consisting of G. vaginalis, Streptococcus agalactiae, Candida albicans, Candida glabrata, Candida parapsilosis, Ureaplasma urealyticum, Escherichia coli, Escherichia coli OBR-01, Klebsiella pneumoniae, Klebsiella pneumoniae K12-4, Klebsiella pneumoniae Kp5, Enterococcus faecalis, Enterococcus faecalis OEA1, Enterococcus faecium, Enterococcus faecium EIPO and any combination thereof, when said activity is determined by the aforementioned co-aggregation assay.

In a particular embodiment, the variant of the isolated strain of the invention maintains the high adhesion to cells and mucus of the vaginal epithelium of the isolated strain of the invention. Adhesion to cells of the vaginal epithelium can be determined by any known adhesion assay, for example, the assay described by Boris et al. (Infect. Immun. 1998, 66, 1985-1989) while adhesion to the mucus can be determined by the assay described by Cohen et al., (Methods Enzymol. 1995, 253, 309-314) which is used in the examples herein employing vaginal epithelial cells and mucus, respectively. In a particular embodiment, the variant maintains the high adhesion to cells of the vaginal epithelium of the isolated strain of the invention when said activity is determined by the co-aggregation assays described by Boris et al. (supra). In a particular embodiment, the variant maintains 100% or more, or at least 99%, at least 98%, at least 97%, at least 96%, at least 95%, at least 94%, at least 93%, at least 92%, at least 91%, at least 90%, at least 85%, at least 80%, at least 75%, at least 70%, at least less 60% or at least 50% of the adhesion of the isolated strain of the invention to cells of the vaginal epithelium, when said activity is determined by the aforementioned adhesion test. In a particular embodiment, the variant maintains the high adhesion to mucus of the isolated strain of the invention when said activity is determined by the co-aggregation assays described by Cohen et al. (supra). In a particular embodiment, the variant maintains 100% or more, or at least 99%, at least 98%, at least 97%, at least 96%, at least 95%, at least 94%, at least 93%, at least 92%, at least 91%, at least 90%, at least 85%, at least 80%, at least 75%, at least 70%, at least less 60% or at least 50% of the adhesion of the isolated strain of the invention to mucus, when said activity is determined by the aforementioned adhesion to the mucus test.

As described in the examples herein, the isolated strain of the invention has an adhesion to vaginal epithelial cells of at least about 343±51 adherent lactobacilli in 20 random microscopic fields when adhesion is determined by the test described by Boris et al. In a particular embodiment the variant of the isolated strain of the invention presents an adhesion of at least about 333±56 adherent lactobacilli in 20 random microscopic fields, or higher when measured by the aforementioned essay.

As described in the examples herein, the isolated strain of the invention has a mean adhesion to mucus of about 11.3±1.6 as determined by the test described by Cohen et al (supra). In a particular embodiment the variant of the isolated strain of the invention presents an adhesion to mucus of at least about 11.3±1.6 when measured by that same essay.

In a particular embodiment, the variant of the isolated strain of the invention maintains the high α-amylase activity of the isolated strain of the invention. The α-amylase activity can be determined by any known assay, as for example, by the method described by Narita et al. (Narita et al. Appl Environ Microbiol. 2006; 72 (1): 269-275) used in the examples, which is based on the determination of the activity on the substrate 2-chloro-4-nitrophenyl 65-azido-65-deoxy-β-maltopentaosideas. In a particular embodiment, the variant maintains the high α-amylase activity of the isolated strain of the invention when said activity is determined by the assay described by Narita et al. (supra). In a particular embodiment, the variant maintains 100% or more, or at least 99%, at least 98%, at least 97%, at least 96%, at least 95%, at least 94%, at least 93%, at least 92%, at least 91%, at least 90%, at least 85%, at least 80%, at least 75%, at least 70%, at least less 60% or at least 50% of the α-amylase activity of the isolated strain of the invention, when said activity is determined by the aforementioned assay. As described in the examples herein, the isolated strain of the invention has an α-amylase activity of 0.80-0.84 U/mL at 16 hours, when said activity is determined by the assay described by Narita et al. In a particular embodiment, the variant of the isolated strain of the invention exhibits an α-amylase activity of about 0. 0.80-0.84 U/ml at 16 hours or higher, when measured by the same assay.

In a particular embodiment, the variant of the isolated strain of the invention maintains the high production of lactic acid of the isolated strain of the invention. In a particular embodiment, the variant of the isolated strain of the invention maintains the high production of lactic acid, in particular of L-lactic acid of the isolated strain of the invention, under aerobic and/or anaerobic conditions. In a particular embodiment, the variant maintains the high production of lactic acid of the isolated strain of the invention when said activity is determined by the assay described in the Examples section. In a particular embodiment, the variant maintains 100% or more, or at least 99%, at least 98%, at least 97%, at least 96%, at least 95%, at least 94%, at least 93%, at least 92%, at least 91%, at least 90%, at least 85%, at least 80%, at least 75%, at least 70%, at least less 60% or at least 50% of the lactic acid production of the isolated strain of the invention, when said production is determined by the aforementioned test. As described in the examples herein, the isolated strain of the invention is capable of producing 10.29±0.53 mg/ml when said production is determined by the test described in the Example section. In a particular embodiment the variant of the isolated strain of the invention is capable of producing an amount of L-lactic acid of at least about 11 mg/ml at both aerobic and anaerobic conditions, or higher when measured by the aforementioned test.

In a particular embodiment, the variant of the isolated strain of the invention is an isogenic mutant, that is, a strain that has a genome that only differs from the L. salivarius CECT 30632 or CECT 30641 strain by the presence of a mutation.

In another particular embodiment, the degree of identity between a variant of the isolated strain of the invention and the parental strain is determined as the average nucleotide identity (ANI), which detects the conservation of the DNA of the central genome (Konstantinidis K and Tiedje J M, 2005, Proc. Natl. Acad. Sci. USA 102:2567-2592). In some embodiments, the ANI between the variant of the isolated strain of the invention and the L. salivarius CECT 30632 or CECT 30641 is about 95%, about 96%, about 97%, about 98%, about 99%, about 99.1%, about 99.5%, of about 99.6%, of about 99.7%, of about 99.8%, of about 99.9%, of about 99.99%, of about 99.999%, of about 99.9999%, of about 99.99999%, about 99.999999% or more but less than 100%.

In another embodiment, the degree of relationship between the variant of the isolated strain of the invention and the L. salivarius CECT 30632 or CECT 30641 is determined as the tetranucleotide signature frequency correlation coefficient, which is based on oligonucleotide frequencies (Bohlin J. et al. 2008, BMC Genomics, 9:104). In some embodiments, the tetranucleotide signature frequency correlation coefficient between the mutant and L. salivarius CECT 30632 or CECT 30641 is approximately 0.99, 0.999, 0.9999, 0.99999, 0.999999, 0.999999 or more, but less than 1.

In another embodiment, the degree of relationship between the variant of the isolated strain of the invention and the L. salivarius CECT 30632 or CECT 30641 is determined as the degree of similarity obtained by analyzing the genomes of the parent and the variant strain by pulsed field gel electrophoresis (PFGE) using one or more endonucleases of restriction. The degree of similarity obtained by PFGE can be measured by the coefficient of similarity of dice. In some embodiments, the given similarity coefficient between the variant of the isolated strain of the invention and the L. salivarius CECT 30632 or CECT 30641 is about 95%, about 96%, about 97%, about 98%, about 99%, about 99, 1%, of about 99.5%, of about 99.6%, of about 99.7%, of about 99.8%, of about 99.9%, of about 99.99%, of about 99.999%, from about 99.9999%, about 99.99999%, about 99.999999% or more but less than 100%.

In another embodiment, a strain is considered a variant of a given parental strain when both strains have the same ribotype, as obtained using any of the methods known in the art and described, for example, by Bouchet et al. (Clin. Microbiol. Rev., 2008, 21:262-273).

In another embodiment, the degree of relationship between the variant of the isolated strain of the invention and the L. salivarius CECT 30632 or CECT 30641 is the Pearson correlation coefficient obtained by comparing the genetic profiles of both strains obtained by repetitive PCR based on extragenic palindromic elements (REP-PCR) (see, for example, Chou and Wang, Int J Food Microbiol. 2006, 110:135-48). In some embodiments, the Pearson correlation coefficient obtained by comparing the REP-PCR profiles of the mutant and the isolated strain of the invention is approximately 0.99, 0.999, 0.9999, 0.99999, 0.999999, 0.999999 or more, but less than 1.

In another embodiment, the degree of relationship between the variant of the isolated strain of the invention and the L. salivarius CECT 30632 or CECT 30641 is the bonding distance obtained by comparing the genetic profiles of both strains obtained by Multilocus sequence sequencing (MLST) (see, for example, Maiden, M C, 1998, Proc. Nati. Acad Sci. USA 95:3140-3145). In some embodiments, the MLST binding distance of the mutant and L. salivarius CECT 30632 or CECT 30641 is about 0.99, 0.999, 0.9999, 0.99999, 0.999999, 0.999999 or more but less than 1.

In another embodiment, the degree of relationship between the variant of the isolated strain of the invention and the L. salivarius CECT 30632 or CECT 30641 is the distance of the variant to the isolated strain of the invention obtained by determining the dendogram of the PCR fingerprinting profiles obtained by random amplification of polymorphic DNA (RAPD), wherein the dendogram distances are calculated using the unweighted pair group method with arithmetic mean (see for example Sharm et al., Food Sci. Biotechnol. 2016 25(6), 1651-1655). In a particular embodiment of the isolated strain of the invention the variant of the isolated strain of the invention has a dendrogram distance to the isolated strain of the invention of less than 10, less than 9, less than 8, less than 7, less than 6, less than 5, less than 4, less than 3, less than 2, less than 1, less than 1, less than 0.9, less than 0.8, less than 0.7, less than 0.6, less than 0.5, less than 0.4, less than 0.3, less than 0.2, less than 0.1, less than 0.1, but more than 0.

In a particular embodiment, the degree of relationship between the variant of the isolated strain of the invention and the L. salivarius CECT 30632 or CECT 30641 is determined by PCR fingerprinting profiles obtained by RAPD, wherein the profiles are obtained through a PCR amplification reaction using a generic primer followed by gel electrophoresis of the PCR amplification product. The term “PCR amplification reaction” as used herein refers to the process of performing a copy of a region of nucleic acids, wherein said copy is multiplied exponentially using oligonucleotides, known usually as “primers”, which are complementary and hybridize with the extremes of said region being amplified. In a particular embodiment the amplification reaction is performed in a thermocycler using the following conditions:
Initial denaturation of 3 minutes at 96° C.,
First amplification phase comprising at least 10 cycles of the following steps:
   Denaturation at 94° C. for 30 seconds;
   Annealing at 30° C. for 30 seconds; and
   Extension at 72° C. for 60 seconds;
Second amplification phase comprising at least 20 cycles of the following steps:
   Denaturation at 94° C. for 30 seconds;
   Annealing at 30° C. for 60 seconds; and
   Extension at 72° C. for 85 seconds;

The term “generic primers” as used herein refers to primers which are shorter, between 8 to 12 base pairs of random sequence, which serve as both forward and reverse primer, and are usually able to amplify fragments at least 5 genomic sites simultaneously.

In a particular embodiment the generic primer is selected from SEQ ID NO: 4, SEQ ID NO: 5, SEQ ID NO: 6 and SEQ ID NO: 7, preferably SEQ ID NO: 4

| SEQ ID NO: | Sequence |
|---|---|
| 4 | ACGCAGGCAC |
| 5 | AACGCGCAAC |
| 6 | CCGCAGCCAA |
| 7 | AACGGGCAGA |

Once the PCR amplification product is obtained the PCR profile is created by running the amplification product in a gel electrophoresis. In a particular embodiment, the electrophoresis is carried out in a 2% agarose gel for 90 minutes at 90 volts.

The isolated strain of the invention L. salivarius CECT 30632 is herein described equally and interchangeably with the acronym “MP98” or “ES27”, while the isolated strain of the invention L. salivarius CECT 30641 is herein describe equally and interchangeably with the acronym “ES43”

In another particular embodiment, the variant of the isolated strain of the invention has a genome that shares a sequence identity of at least 90%, 91%, 92%, 93%, 94%, 95%, 96%, 97%, 98% or 99% with the genome of the L. salivarius CECT 30632. In another particular embodiment, the variant of the isolated strain of the invention has a genome that shares a sequence identity of at least 90%, 91%, 92%, 93%, 94%, 95%, 96%, 97%, 98% or 99% with the genome of the L. salivarius CECT 30641. In another particular embodiment, the variant of the of the L. salivarius CECT 30632 is characterized by the presence of a 16S rRNA gene that has at least 97%, at least 98%, at least 99%, at least 99.5% or at least 99.7%, or at least 99.8%, or at least 99.9% sequence identity with SEQ ID NO: 1

SEQ ID NO: 1
GACAGTTACTCTCACTCGTGTTCTTCTCTAACAACAGAGTTTTACGATCCGAAGAC
CTTCTTCACTCACGCGGCGTTGCTCCATCAGACTTGCGTCCATTGTGGAAGATTC
CCTACTGCTGCCTCCCGTAGGAGTTTGGGCCGTGTCTCAGTCCCAATGTGGCCG
ATCAACCTCTCAGTTCGGCTACGTATCATCACCTTGGTAGGCCGTTACCCCACCA
ACTAGTTAATACGCCGCGGGTCCATCTAAAAGCGATAGCAGAACCATCTTTCATCT
AAGGATCATGCGATCCTTAGAGATATACGGTATTAGCACCTGTTTCCAAGTGTTAT
CCCCTTCTTTTAGGCAGGTTACCCACGTGTTACTCACCCGTCCGCCACTCAACTTC
TTACGGTGAATGCAAGCATTCGGTGTAAGAAAGTTTCGTTCGACTTGCATGTATTA
GGCACGCCGCCAGCGTTCGTCCTGAGCCAGGNTCAAACTCTAN

In another particular embodiment, the variant of the of the L. salivarius CECT 30632 is characterized by the presence of a 16S rRNA gene that has at least 97%, at least 98%, at least 99%, at least 99.5% or at least 99.7%, or at least 99.8% or at least 99.9% sequence identity with SEQ ID NO: 2

SEQ ID NO. 2
TAGAAAGGAGGTGATCCAGCCGCAGGTTCTCCTACGGCTACCTTGTTACGACTTC
ACCCCAATCATCTGTCCCACCTTAGACGGCTGGCTCCTTGCGGTTACCCCACCGG
CTTTGGGTGTTACAAACTCTCATGGTGTGACGGGCGGTGTGTACAAGGCCCGGG
AACGTATTCACCGCGACATGCTGATTCGCGATTACTAGCGATTCCGACTTCATGTA
GGCGAGTTGCAGCCTACAATCCGAACTGAGAACGGCTTTAAGAGATTAGCTAAAC
CTCGCGGTCTCGCGACTCGTTGTACCGTCCATTGTAGCACGTGTGTAGCCCAGGT
CATAAGGGGCATGATGACTTGACGTCGTCCCCACCTTCCTCCGGTTTGTCACCGG
CAGTCTCGCCAGAGTGCCCAACTTAATGCTGGCAACTGACAACAAGGGTTGCGCT
CGTTGCGGGACTTAACCCAACATCTCACGACACGAGCTGACGACAGCCATGCAC
CACCTGTCACTTTGTCCCCGAAGGGAAAGCCTAATCTCTTAGGTGGTCAAAGGAT
GTCAAGACCTGGTAAGGTTCTTCGCGTTGCTTCGAATTAAACCACATGCTCCACC
GCTTGTGCGGGCCCCCGTCAATTCCTTTGAGTTTCAACCTTGCGGTCGTACTCCC
CAGGCGGAATGCTTATTGCGTTAGCTGCGGCACTGAAGGGCGGAAACCCTCCAA
CACCTAGCATTCATCGTTTACGGCGTGGACTACCAGGGTATCTAATCCTGTTTGCT
ACCCACGCTTTCGAACCTCAGCGTCAGTTACAGACCAGAGAGCCGCTTTCGCCAC
TGGTGTTCTTCCATATATCTACGCATTTCACCGCTACACATGGAGTTCCACTCTCC
TCTTCTGCACTCAAGTCTTCCAGTTTCCAATGCACTACTCCGGTTAAGCCGAAGGC
TTTCACATCAGACTTAAAAGACCGCCTGCGTTCCCTTTACGCCCAATAAATCCGGA
CAACGCTTGCCACCTACGTATTACCGCGGCTGCTGGCACGTAGTTAGCCGTGACT
TGCTGGTTAGATACCGTCATCGAATGAACAGTTACTCTCACTCGTGTTCTTCTCTA
ACAACAGAGTTTTACGATCCGAAGACCTTCTTCACTCACGCGGCGTTGCTCCATC
AGACTTGCGTCCATTGTGGAAGATTCCCTACTGCTGCCTCCCGTAGGAGTTTGGG
CCGTGTCTCAGTCCCAATGTGGCCGATCAACCTCTCAGTTCGGCTACGTATCATC
ACCTTGGTAGGCCGTTACCCCACCAACTAGTTAATACGCCGCGGGTCCATCTAAA
AGCGATAGCAGAACCATCTTTCATCTAAGGATCATGCGATCCTTAGAGATATACGG
TATTAGCACCTGTTTCCAAGTGTTATCCCCTTCTTTTAGGCAGGTTACCCACGTGT
TACTCACCCGTCCGCCACTCAACTTCTTACGGTGAATGCAAGCATTCGGTGTAAG
AAAGTTTCGTTCGACTTGCATGTATTAGGCACGCCGCCAGCGTTCGTCCTGAGCC
AGGATCAAACTCTCATTTTAAAGTTTGTGA

In a preferred embodiment, the sequence identity is determined throughout the whole length of the SEQ ID NO: 1 or 2. In another preferred embodiment, the sequence identity is determined throughout the whole length of the 16S rRNA gene of the variant.

In another particular embodiment, the variant of the L. salivarius CECT30641 is characterized by the presence of a 16S rRNA gene that has at least 97%, at least 98%, at least 99%, at least 99.5% or at least 99.7%, or at least 99.8% or at least 99.9% sequence identity with SEQ ID NO: 3.

SEQ ID NO: 3
ACCTTATGACAATTCTCATCACTCGTGTTCTTCTCTAACAACAGAGTTTTACGATCC
GAAGACCTTCTTCACTCACGCGGCGTTGCTCCATCAGACTTGCGTCCATTGTGGA
AGATTCCCTACTGCTGCCTCCCGTAGGAGTTTGGGCCGTGTCTCAGTCCCAATGT
GGCCGATCAACCTCTCAGGTCGGCTACGTATCATCACCTTGGTAGGCCGTTACCC
CACCAACTAGTTAATACGCCGCGGGGACCTCTAAAAGCGATAGCAGAAACCTCTT
TCTTCTAAGGAACCCGCGATCCTCAAAAG

In a preferred embodiment, the sequence identity is determined throughout the whole length of the SEQ ID NO: 3. In another preferred embodiment, the sequence identity is determined throughout the whole length of the 16S rRNA gene of the variant.

The terms “identity”, “identical” or “percent identity” in the context of two or more amino acid or nucleotide sequences, refer to two or more sequences or subsequences that are the same or have a specified percentage of nucleotide or amino acid residues that are the same, when compared and aligned (introducing gaps, if necessary) for maximum correspondence, not considering any conservative substitutions as part of the sequence identity. The percent identity can be measured using sequence comparison software or algorithms or by visual inspection. Various algorithms and software are known in the art that can be used to obtain alignments of amino acid or nucleotide sequences. Publicly available software programs can be used to align sequences. Appropriate parameters for maximal alignment by particular alignment software can be determined by one skilled in the art. In certain embodiments, the default parameters of the alignment software are used. In certain embodiments, the percentage identity “X” of a first nucleotide sequence to a second nucleotide sequence is calculated as 100×(Y/Z), where Y is the number of nucleotide residues scored as identical matches in the alignment of the first and second sequences (as aligned by visual inspection or a particular sequence alignment program) and Z is the total number of residues in the second sequence. If the second sequence is longer than the first sequence, then the global alignment taken the entirety of both sequences into consideration is used, therefore all letters and null in each sequence must be aligned. In this case, the same formula as above can be used but using as Z value the length of the region wherein the first and second sequence overlaps, said region having a length, which is substantially the same as the length of the first sequence.

For instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

The isolated strain of the invention, according to the invention can be provided as a culture. As such, another aspect of the present invention relates to a bacterial culture comprising the isolated strain of the invention, from here onwards the bacterial culture of the invention.

The term “bacterial culture” in the context of the present invention refers to a population of living bacterial cells. Several types of culture and culture conditions are part of the expert general knowledge, which would have any difficulty in setting up and cultivating the strain of the invention. Several types of culture are known in the art, such as liquid culture or broth culture, agar plate cultures, agar-based dipsticks and stab cultures. The cultures are grown according to their requirements. In a particular embodiment of the bacterial culture of the invention, the bacterial culture is grown on MRS agar medium. In another particular embodiment of the MRS agar is supplemented with L-cysteine or horse blood, preferably from about 1.5 g/L to about 3.5 g/L of L-cysteine or from about 2.5% weight per volume (w/v) to about 7.5% w/v horse blood. In another particular embodiment of the bacterial culture of the invention, the bacterial culture is incubated at a temperature from about 30° C. to about 42° C., preferably 37° C., during from about 24 h to about 72 h, preferably 48 h. In a particular embodiment of the bacterial culture of the invention, the culture is incubated in anaerobic conditions, wherein the incubation environment comprises from about 80% to about 90% nitrogen, 5% to about 15% hydrogen and 1% to about 10% carbon dioxide, wherein the sum of the gas percentages is 100%.

The bacterial culture of the invention can be preserved and presented in a solution, suspension, powder, lyophilized, etc. In a particular embodiment of the bacterial culture of the invention, the bacterial culture is lyophilized.

As used herein, the term “freeze dried/lyophilized” refers to a laboratory method where live microbes in aqueous suspension are rapidly frozen to <50° C., and then the majority of the frozen water content is forced to sublime under vacuum conditions, allowing this water to be efficiently removed in the gaseous phase. Protocols for the lyophilization of bacterial cultures are known in the art such as, without limitation, the protocol disclosed by (Shekh et al., Ann Microbiol 2020, 70, 16). In a particular embodiment of the culture of the invention, the bacterial culture is lyophilized in the presence of wall materials selected from a group consisting of: fructooligosaccharide, inulin, lactulose, sucrose, fructooligosaccharide and skim milk, inulin and skim milk, lactulose and skim milk, sucrose and skim milk and skim milk. In a more particular embodiment of the bacterial culture of the invention, the bacterial culture is lyophilized in the presence of skim milk.

The term “wall material” as used herein refers to the material or substance used in the process of the lyophilization that is used to encapsulate or trap the microorganism to be encapsulated (in the present case the isolated strain of the invention). This material is also called the encapsulation material, cover membrane, shell, vehicle, or external phase matrix.

### Compositions of the Invention

All the terms, definitions, and embodiments previous disclosed in relation to previous aspects are equally valid for the present aspects and their embodiments.

The isolated strain of the invention and the bacterial culture of the invention can be provided without any other components or in compositions that are directed to certain purposes. As such, another aspect of the present invention relates to a pharmaceutical composition, from here onwards the pharmaceutical composition of the invention, comprising a therapeutic effective dose of the isolated strain of the invention or the bacterial culture of the invention and an acceptable adjuvant.

The expression “therapeutic effective dose” as used herein refers to an amount of an active agent (i.e., an ingredient such as a bacterial strain, in the present invention is the strain L. salivarius CECT 30632 or a variant thereof or the strain L. salivarius CECT 30641 or a variant thereof) high enough to deliver the desired benefit, either the treatment or prevention of the illness, but low enough to avoid serious side effects within the scope of medical judgment. The particular dose of compound administered according to this invention will of course be determined by the particular circumstances surrounding the case, including the compound administered, the route of administration, the particular condition being treated, and the similar considerations. In a particular embodiment of the pharmaceutical composition of the invention the therapeutic effective dose from about 8 log 10 colony forming units (CFU) to about 10 log 10 CFU, preferably 9 log 10 CFU.

The expression “colony forming units” as used herein refers to an estimation of the number of bacteria or fungal cells in a sample that are viable, able to multiply via binary fission under the controlled conditions. The number of viable cells present in a sample can be multiplied by a diluting factor to obtain CFU/mL. In addition, the weight of sample can be measured to obtain a measurement in mg/mL.

The pharmaceutical composition can be formulated as a solution, a suspension, a tablet, a cream, etc. In a particular embodiment, the pharmaceutical composition of the invention is formulated for oral, vaginal, intramammary, rectal, anal, intravenous, intraarterial, parenteral, intracranial, topical, respiratory or nasal administration.

In a more particular embodiment, the pharmaceutical composition of the invention can be formulated as a topical composition. Said topical pharmaceutical compositions can be adapted to apply to the skin and mucosa in the form of: a non-ionic vesicular dispersion, emulsion, cream, lotion, gel, aerosol, cream-gel, gel-cream, suspension, dispersion, powder, solid stick, wipe, poultice, foam, spray, oil, ointment, fluid, soap, sanitary towel, ovule, pessary tampon, vaginal suppository or any other form that is known in the art of cosmetics and pharmacy. In a particular embodiment, the pharmaceutical composition of the invention is formulated as a gel and administered vaginally or cervically by means of a cannula. In another particular embodiment, the pharmaceutical composition of the invention is fomrulated as a vaginal suppository or ovule for vaginal administation. In a more particular embodiment, the pharmaceutical composition of the invention is for genital administration, particularly to the vulva, vagina or cervix.

The term “adjuvant” as used herein refers to any substance that enhances the response of a given substance. In the present invention, said term refers to any substance that enhances the effects of the pharmaceutical composition of the invention; it can refer to any adjuvant known by the person skilled in the art.

In a particular embodiment of the pharmaceutical composition of the invention, the composition further comprises a component selected from a group consisting of: prebiotic, vitamin, mineral, metal, oligoelement, plant-derived component, fungal-derived component, carotenoid, anti-oxidant, or any combination thereof.

As used herein, the term “prebiotic” includes substances or compounds that beneficially affect the host mammal by selectively promoting the growth and/or activity of one or more probiotic bacteria in the gastrointestinal tract of the host mammal, thus maintaining normal health or improving health of the host. Typically, prebiotics are carbohydrates, (such as oligosaccharides), but the term “prebiotic” as used herein does not preclude non-carbohydrates (e.g., polyphenols).

Non-limiting examples of prebiotics suitable for use in the pharmaceutical composition of the invention include inulin, psyllium, fructo-oligosaccharides, oligofructose, galacto-oligosaccharides, isomalto-oligosaccharides xylo-oligosaccharides, soy-oligosaccharides, gluco-oligosaccharides, mannan-oligosaccharides, arabinogalactan, arabinxylan, lactosucrose, gluconannan, lactulose, polydextrose, oligodextran, gentioligosaccharide, pectic oligosaccharide, xanthan gum, gum arabic, hemicellulose, resistant starch and its derivatives, and mixtures and/or combinations thereof.

The isolated strain of the invention can, as described in the Examples section below, improve the vaginal microbiota and regulate its parameters. Therefore, another aspect of the present invention relates to a cosmetic composition having an effect of reducing vaginal discharge comprising the isolated strain of the invention or the bacterial culture of the invention.

The term “cosmetic composition” as used herein refers to substances or preparations intended to come into contact with various surface parts of the human body, in particular the epidermis, nails and lips, and mucosa membranes, solely or mainly for cleaning them, making them fragrant, changing their appearance, protect them, keep them in good shape, or correct body odor. Cosmetic compositions are intended for non-therapeutic use.

The term “vaginal discharge” as used herein refers to the secretion of fluid by the glands inside the vagina and cervix. Excess vaginal discharge can occur as a result of arousal, ovulation, infections, hormone imbalance or microbiota imbalance. Normal vaginal discharge ranges in color from clear or milky to white. The consistency of vaginal discharge also varies from thin and watery to thick and sticky. Generally, healthy vaginal discharge should be relatively odorless. Elevated vaginal discharge can be associated with an unpleasant odor. The expression “reducing vaginal discharge” refers to the effect caused by the cosmetic composition of reducing the amount of vaginal fluid secreted due to correction of the microbiota imbalance and improvement of the vaginal ecosystem environmental parameters, such as pH, hormonal levels, etc.

### Medical Uses of the Invention

All the terms, definitions, and embodiments previous disclosed in relation to previous aspects are equally valid for the following aspects and their embodiments.

The role of estrogens in the development and function of the female reproductive tract and the effects on non-reproductive organs (e.g. gut, brain, bones, mammary glands) and tissues (e.g. adipose tissue) are kwnon. Estrogens participate, directly or indirectly, in several human pathologies including infectious, autoimmune, metabolic, oncologic and degenerative diseases.

As previously mentioned the isolated strain of the invention has specific characteristics, which make it useful for the prevention and/or treatment of certain medical conditions. Therefore, another aspect of the present invention relates to the isolated strain of the invention or the bacterial culture of the invention for use as a medicament or for use in medicine.

In an aspect the invention relates to the isolated strain according to the invention, the bacterial culture according to the invention or the pharmaceutical composition according to the invention for use in the prevention and/or treatment of a disease associated with high levels of estrogens.

Alternatively, the invention relates to a method for preventing and/or treating a a disease associated with high levels of estrogens which comprises administering the isolated strain according to the invention, the bacterial culture according to the invention or the pharmaceutical composition according to the invention to a subject in need thereof.

In an aspect the invention relates to the isolated strain according to the invention, the bacterial culture according to the invention or the pharmaceutical composition according to the invention for use in the prevention and/or treatment of a disease associated with altered levels of estrogens. In a preferred embodiment, the altered levels of estrogens are high levels. In a nother preferred embodiment, the altered levels are low levels of estrogens.

Alternatively, the invention relates to a method for preventing and/or treating a a disease associated with altered levels of estrogens which comprises administering the isolated strain according to the invention, the bacterial culture according to the invention or the pharmaceutical composition according to the invention to a subject in need thereof. In a preferred embodiment, the altered levels of estrogens are high levels. In a nother preferred embodiment, the altered levels are low levels of estrogens.

The term “prevention”, as used herein, relates to the capacity to prevent, minimize, or hinder the onset or development of a disease or condition before its onset.

As used herein, the terms “treat”, “treatment”, “treatment”, or “amelioration”. The term refers to therapeutic treatment, the purpose of which is to reverse, reduce, suppress, delay or stop the progression or severity of the condition associated with the disease or disorder. The term “treatment” includes reducing or alleviating at least one adverse effect or condition of a condition, a disease or disorder, such as an infection. Treatment is usually “effective” when one or more symptoms or clinical markers are reduced. Alternatively, treatment is “effective” if disease progression is delayed or halted. That is, “treatment” includes not only the improvement of symptoms or markers, but also the interruption of at least a condition that indicates the progression or worsening of symptoms that would be expected in the absence of treatment. The beneficial or desirable clinical outcome, whether detectable or not, is a reduction in one or more symptoms, a reduction in the extent of the disease, a stable (ie, not aggravated) condition of the disease. These include, but are not limited to, delayed or slowed progression, amelioration or alleviation of the disease state, and remission (partial or total). The term “treatment” of a disease also includes providing relief from symptoms or side effects of the disease (including symptomatic treatment).

The term “subject” or “patient”, as used herein, refers to all animals classified as mammals and includes, but is not restricted to, domestic and farm animals, primates and humans, e.g., human beings, non-human primates, cows, horses, pigs, sheep, goats, dogs, cats or rodents. Preferably, the subject is a male or female human of any age or race, more preferably a female.

“Altered levels of estrogens”, as used herein relates to the level of a particular estrogen which is at least 5%, 10%, 20%, 30%, 40% or more higher or at least least 5%, 10%, 20%, 30%, 40% or more lower than the normal level of said estrogen.

“Disease associated with high levels of estrogen”, as used herein relates to any disease or disorder caused by estrogen imbalance. Estrogen levels vary among individuals and they also fluctuate during the menstrual cycle and over a female's lifetime. The normal levels for each estrogen are widely known. For example normal levels of estradiol (E2) for menstruating women range from 15 to 350 picograms per milliliter (pg/mL). “High leves of estrogen”, as used herein relate to the level of a particular estrogen which is at least 5%, 10%, 20%, 30%, 40% or more higher than the normal level of said estrogen.

In a preferred embodiment, the disease associated with high levels of estrogen is selected from a group consisting of endometriosis, infertility, gynecomastia, erectile dysfunction, fibrocystic breast disease, hypoactive sexual desire disorder, menorrhagia, premenstrual syndrome, obesity, menopause associated hair loss and sleeping disorder.

In a preferred embodiment the disease is endometriosis. “Endometriosis”, as used herein relates to a condition wherein tissue similar to the lining of the uterus grows in other places within your abdomen and pelvic area.

In another preferred embodiment, the disease is estrogen-dependent cancer.

“Estrogen-dependent cancer”, as used herein relates to a tumor expressing estrogen receptors and/or the development of which can be stimulated by estrogens

In a preferred embodiment, the estrogen dependent cancer is seslected from the group consisting of breast cancer, ovarian cancer an endometrial cancer. In a more preferred embodiment, the cancer is breast cancer, more particularly estrogen receptor (ER) positive breast cancer.

In another aspect, the present invention relates to the isolated strain of the invention, the bacterial culture of the invention or the pharmaceutical composition of the invention for use in the prevention and/or treatment of an infectious disease, from here onwards the infectious treatment use of the invention.

Alternatively, the invention relates to a method for the prevention and/or treatment of an infectious disease which comprises administering the isolated strain of the invention, the bacterial culture of the invention or the pharmaceutical composition of the invention to a subject in need thereof.

The term “infectious disease” as used herein refers to the invasion of an organism's body tissues by disease-causing agents, their multiplication, and the reaction of host tissues to the infectious agents and the toxins they produce. An infectious disease, also known as a transmissible disease or communicable disease, is an illness resulting from an infection. Depending on where or which tissue is infected the disease can take up several names. In a particular embodiment of the infectious treatment use of the invention, the infection disease is selected from a group consisting of: urinary tract infection (infection in any part of your urinary system-kidneys, ureters, bladder and urethra), gynecological infection (infections affecting the vagina and genital tract), oral thrush (infection of the lining of the mouth), throat thrush (infection of the esophagus lining), skin and soft tissue infection, sepsis (generalized host infection), neonatal sepsis (generalized host infection in preterm babies), early onset sepsis (generalized host infection in the early weeks of babies), bacteremia (presence of bacteria in the blood), fungemia (presence of fungi in the blood), meningitis (infection of the protective membranes of the brain and spinal cord), neonatal meningitis (infection of the protective membranes of the brain and spinal cord in preterm babies), osteomyelitis (infection of the bone), mastitis (infection of the breast), endometritis (infection of the inner lining of the uterus), endocarditis (infection of the inner layer of the heart), pulmonary infection (infection of the lungs), pneumonia (infection of the one or more sections of the lobes of the lungs), bronchopneumonia (pneumonia that causes inflammation of the alveoli), bronchitis (infection of the bronchi of the lungs), sinusitis (infection of the spaces inside the nose and head-sinuses), tonsillitis (infection of the tonsils) and laryngitis (infection of the voice box-larynx). In a preferred embodiment, the infection is a vaginal infection.

Infections can be caused by a wide range of pathogens, most prominently bacteria, viruses and fungus. Said microorganisms can be foreign to the host or a pre-existent microorganism in the host, which explores a temporary opportunity to infect the host. In a particular embodiment of the infectious treatment use of the invention, the infectious disease is a fungal disease, a bacterial disease and/or a virus infection.

In a more particular embodiment of the infectious treatment use of the invention the fungal infection is caused by a fungus selected from a group consisting of: Candida albicans, Candida glabrata, Candida parapsilosis or any combination thereof. Infections caused by Candida species are also called candidiasis.

As used herein the term “candidiasis” refers to a fungal infection due to any type of Candida (a genus of yeasts). When it affects the mouth, it is commonly called thrush. Signs and symptoms include white patches on the tongue or other areas of the mouth and throat. Other symptoms may include soreness and problems swallowing. When it affects the vagina, it may be referred to as a yeast infection or vaginal thrush. Signs and symptoms include genital itching, burning, and sometimes a white “cottage cheese-like” discharge from the vagina. Yeast infections of the penis are less common and typically present with an itchy rash. Very rarely, yeast infections may become invasive, spreading to other parts of the body. This may result in fevers along with other symptoms depending on the parts involved. More than 20 species of Candida can cause infection with Candida albicans being the most common. The second most common cause of candidiasis is C. glabrata, which normally infects the urogenital track. C. glabrata is innately highly resistance to antifungal agents, specifically the azoles, which make it of specially concern in immunocompromised patients. Likewise, C. parapsilosis has become a significant cause of sepsis and of wound and tissue infections in immunocompromised people, which make it an important target for new methods of treatment.

In another particular embodiment of the infectious treatment use of the invention the infection is an urinary tract infection and the infection is caused by an uropathogen.

The term “uropathogen” as used herein refers to pathogens, usually gram-negative bacteria, which have evolved to being capable of causing disease of the urinary tract. Uropathogens differ from other pathogens in term of the virulence factors and pathogenic mechanisms that allow them to colonize and infect the urinary tract. An example of such differences is the presence of the enzyme urease, which hydrolyzes urea to ammonia and carbon dioxide. In a preferred embodiment the uropathogen is selected from a group consisting of: Actinotignum schaalii, Aerococcus sanguinicola, Aerococcus urinae, Alloscardovia omnicolens, Candida albicans, Candida parapsilosis, Citrobacter freundii, Citrobacter koseri, Corynebacterium riegelii, Corynebacterium urealyticum, Enterobacter aerogenes, Enterococcus faecalis, Escherichia coli, Klebsiella pneumoniae, Morganella morganii, Oligella urethralis, Proteus mirabilis, Pseudomonas aerugmosa, Serratia marcescens, Staphylococcus aureus, Staphylococcus lugdunensis, Streptococcus agalactiae and any combination thereof.

In another particular embodiment of the infectious treatment use of the invention the bacterial infection is caused by a bacteria selected from the group consisting of: Streptococcus agalactiae, Gardnerella vaginalis, Ureaplasma urealyticum, Escherichia coli, Escherichia coli OBR-01, Klebsiella pneumoniae, Klebsiella pneumoniae K12-4, Klebsiella pneumoniae Kp5, Enterococcus faecalis, Enterococcus faecalis OEA1, Enterococcus faecium, Enterococcus faecium EIPO and any combination thereof.

The term “Streptococcus agalactiae” (also known as group B streptococcus or GBS) is a gram-positive coccus with a tendency to form chains (as reflected by the genus name Streptococcus). It is a beta-hemolytic, catalase-negative, and facultative anaerobe. S. agalactiae is the most common human pathogen of streptococci belonging to group B of the Rebecca Lancefield classification of streptococci. In general, GBS is a harmless commensal bacterium being part of the human microbiota colonizing the gastrointestinal and genitourinary tract of up to 30% of healthy human adults (asymptomatic carriers). Nevertheless, GBS can cause severe invasive infections especially in newborns, the elderly, and people with compromised immune systems. S. agalactiae is also a common veterinary pathogen, because it can cause bovine mastitis (inflammation of the udder) in dairy cows.

The term “Gardnerella vaginalis” in the context of the present invention refers to a Gram-variable-staining facultative anaerobic bacteria belonging to the Gardnerella genus of which Gardnerella vaginalis is the only species. G. vaginalis is involved, together with many other bacteria, mostly anaerobic, in bacterial vaginosis in some women as a result of a disruption in the normal vaginal microflora.

The term “Ureaplasma urealyticum” as used herein refers to a bacterium belonging to the genus Ureaplasma and the family Mycoplasmataceae. This bacterium is commonly found in the urogenital tracts of human beings, but overgrowth can lead to infections that cause the patient discomfort. Unlike most bacteria, Ureaplasma urealyticum lacks a cell wall making it unique in physiology and medical treatment. Ureaplasma urealyticum can cause urethritis and may cause bacterial vaginosis. Infection can occur in extragenital sites. A common symptom associated with these infections is the “fishy” smell that is created due to the production of ammonia and related compounds (e.g. trimethylamine or TMA) by the hydrolysis or urea.

The term “Escherichia coli” as used herein refers to a Gram-negative, facultative anaerobic, rod-shaped, coliform bacterium of the genus Escherichia that is commonly found in the lower intestine of warm-blooded organisms. Most E. coli strains are harmless, but some serotypes (EPEC, ETEC etc.) can cause serious food poisoning in their hosts.

The term “Klebsiella pneumoniae” as used herein refers to a Gram-negative, non-motile, encapsulated, lactose-fermenting, facultative anaerobic, rod-shaped bacterium. Usually found in the normal microbiota of the mouth, skin and intestines, it can infect the lung tissue and cause destructive changes if aspirated. The term “Enterococcus faecalis” in the context of the present invention relates to a Gram-positive, commensal bacterium inhabiting the gastrointestinal and genitourinary tracts of humans. E. faecalis can be an opportunistic pathogen, especially in nosocomial environments.

The term “Enterococcus faecium” in the present context refers to a Gram-positive, gamma-hemolytic or non-hemolytic bacterium in the genus Enterococcus It can be commensal (innocuous, coexisting organism) in the gastrointestinal and genitourinary tracts of humans and animals, but it may also be pathogenic, causing diseases such as neonatal meningitis or endocarditis.

In a particular embodiment of the infectious treatment use of the invention, the infection is a viral infection, in particular coronavirus disease 2019 (COVID-19).

The term “coronavirus disease 2019” or its acronym “COVID-19” as used herein refer to the illness caused by infection with the severe acute respiratory syndrome coronavirus 2 (SARS-COV-2), which causes several symptoms, including fever, cough, headache, fatigue, breathing difficulties, loss of smell, and loss of taste.

In another aspect, the present invention relates to the isolated strain of the invention, the bacterial culture of the invention or the pharmaceutical composition of the invention for use in the prevention and/or treatment of an inflammatory disease, from here onwards the inflammatory use of the invention.

Alternatively, the invention relates to a method for preventing and/or treating an inflammatory disease, which comprises administering the isolated strain of the invention, the bacterial culture of the invention or the pharmaceutical composition of the invention to a subject in need thereof.

The term “inflammatory disease” as used herein refers to lesions caused by a defensive reaction or an inflammatory reaction of a living body against harmful influence of circumstances (such as physical, chemical and biological circumstarices) having signs of redness, heat, pain, swelling and loss of function. The inflammatory diseases includes conjunctivitis, iritis, uveitis, central retinitis, external otitis, acute suppurative otitis media, mastoiditis, labyrinthitis, chronic rhinitis, acute rhinitis, sinusitis, pharyngitis, tonsillitio, chronic bronchitis, acute bronchilotis, lobar pneumonia, bronchopneumonia, primary atypical pneumonia, dry pleurisy, wet pleurisy, mediastinitis, acute rheumatic endocarditis, bacterial endocarditis, thrombophlebitis, polyarteritis, acute nephritis, chronic nephritis, cystitis, paranephlitis, stomatitis, esophagitis, acute gastritis, chronic gastritis, ulcertive colitis, acute appendicitis, chronic hepatitis, acute hepatitis, cholangiolitic hepatitis, cholecystitis, chronic pancreatitis, acute pancreatitis, chronic peritonitis, acute peritonitis, thyroiditis, contact dermatitis, acute hemorrhagic encephalitis, purulent meningitis, optic neuromyelitis, alcohlic polyneuritis, diabetic polyneuritis, polymyositis, myositis ossificans, degenerative arthritis, gout, rheumatoid arthritis, periarthritis scapulohumeralis, osteitis deformans, etc.

In a particular embodiment of the inflammatory use of the invention, the inflammatory disease is arthritis and/or gout.

The term “arthritis” in the present context refers to a group of related disorders of the joints which are characterized by pain, inflammation and stiffness. Certain types of arthritis are due to specific infectious agents, but no specific cause is known for the most common type, known as rheumatoid arthritis. While this is primarily an inflammatory disease of the joints, it is sometimes accompanied by pedal edema, that it, an accumulation of fluid in the tissues of the foot. Treatment of this disease involves the administration of agents, which have been found effective in reducing the pain and the inflammation. Such agents have come to be generally known as anti-inflammatory agents.

The term “gout” as used herein refers to a crystal-associated arthropathy caused by monosodium urate (MSU) deposition, and is directly related to hyperuricemia caused by purine metabolism disorders, or/and hyperuricemia caused by decreased uric acid excretion. Gout may present several symptoms, the most common of which is acute gouty arthritis, and the typical attack is characterized by sudden red, swollen and heat pain in the involved joints. The most common involved joints are the first metatarsophalangeal joints, the ankle, knee and tarsal joints. Long-term gout may lead to tophus deposition, joint destruction, loss of function, and disappearance of diapause, culminating into chronic tophaceous gout, urate nephropathy and renal insufficiency.

Treatments for patients affected by hyperuricemia causing gout are based on modulating the activity of key enzymes involved in metabolism and excretion of uric acid. There are two main classes: uricostatic drugs (e.g. allopurinol and febuxostat), the most frequently used, that through a competitive inhibition of xantine oxidase reduce uric acid production, and uricosuric drugs (e.g. sulphinpyrazone, probeneceid and benzbromarone) which increase urinary uric acid excretion by blocking renal tubular reabsorption of urate.

The number of patients in gout therapy achieving serum urate levels <6 mg/dl (<0.35 mmol/L) is in the range of 20-40% for allopurinol and 45-67% for febuxostat. Uricosuric drugs (URAT1 inhibitors) are usually used as an option when the patients are intolerant to allopurinol

Another aspect of the present invention relates to the isolated strain of the invention, the bacterial culture of the invention or the pharmaceutical composition of the invention for use in the prevention and/or treatment of disease or syndrome selected from hyperuricemia or associated syndromes, high cholesterol, high triglycerides and oxidative stress.

Alternatively, the invention relates to a method for preventing and/or treating a disease or syndrome selected from hyperuricemia or associated syndromes, high cholesterol, high triglycerides and oxidative stress, which comprises administering the isolated strain of the invention, the bacterial culture of the invention or the pharmaceutical composition of the invention to a subject in need thereof.

The term “hyperuricemia” as used herein refers to disease, disorder or condition characterized by elevated (i.e. above normal) levels serum uric acid. Suitably examples include, but are not limited to gout, urate nephropathy, chronic kidney disease, hypertension, and kidney stones. Preferably, the hyperuricemia or related disorder” is selected from the group consisting of urate nephropathy, chronic kidney disease, hypertension, and kidney stones. Hyperuricemia is defined as a serum uric acid level higher than 7.0 mg/dl in men and 6.0 mg/dl in women, measured at 37° C. and neutral pH. Although there is a considerable regional variation, hyperuricemia is patent in all populations where it has been studied. Some factors have an important role on the development of hyperuricemia such as sex, age, genetics, race or ethnicity. Sex seems to be an important non-modifiable risk factor as females have lower serum UA level than males. The explanation behind this fact is the oestrogen's enhancement of renal tubular urate excretion leading to the reduced risk of hyperuricemia and incident gout in pre-menopausal females. However, growing evidence suggest a substantial increase of hyperuricemia also among women.

Different causes leads to hyperuricemia, being the most important the overproduction of uric acid, a dysfunction of its excretion by kidney disease, or both. However, there is a long-held association of hyperuricemia with some dietary factors. Among them, alcohol and high meat or seafood intake can be considered the most classical factors. Most recently, an association with diets containing high-purine vegetables has also been reported. Other dietary factors that contribute to hyperuricemia besides purine-rich foods are fructose/sugar-sweetened beverages, dairy products, and coffee.

The clinical spectrum associated to hyperuricemia ranges from the classic presentation of episodic and acute inflammation of the first metatarsophalangeal joint to tophaceous gout, chronic polyarticular arthritis, urate nephrolithiasis and interstitial nephropathy. Among them, the most known disease related to hyperuricemia is gout, an inflammatory arthritis triggered by the crystallization of uric acid within the joints when the blood level of uric acid reaches its physiologic limit of solubility. Gout affects at least 1% of the population in Western countries.

Furthermore, uric acid may predict the development of metabolic syndrome, obesity and diabetes. Recently, different studies have defined hyperuricemia as a risk factor for diseases such as arteriosclerosis, cerebrovascular and cardiovascular disease, chronic kidney disease, and nephropathy in diabetic patients. This condition is commonly observed in patients with metabolic syndrome together with diabetes, stroke, and coronary heart disease. Patients with hyperuricemia were found to be at a significantly higher risk for both stroke incidence (relative risk 1.41) and mortality (relative risk 1.26) than controls with normal levels of uric acid. Increased uric acid levels were found to be associated with increased risk of both hemorrhagic and ischemic strokes.

It has been proved that abnormal uric acid level induces endothelial dysfunction and renal fibrosis. Some studies have suggested that uric acid has also a direct influence in the development of neurodegenerative diseases. In this case, low levels lead to the onset of degenerative diseases as Alzheimer's disease, Huntington's disease, Parkinson's disease, and multiple sclerosis.

“Uric acid” or “UA” (UA; 7,9-dihydro-1H-purine-2,6,8(3H)-trione) as used herein refers to a heterocyclic organic compound with a molecular weight of 168 Da and the general formula C₅H₄N₄O₃. UA is a weak dibasic acid with dual pKa of 5.8 and 10.3, although in the human body under physiologic conditions (pH 7.4 and 37° C.) it is mostly present in the monodeprotonated ionic form. The divalent urate anion is almost absent in the human body because of the high second pKa, and in the biomedical literature the term urate ion is used to refer to the monovalent urate. Furthermore, the terms UA and urate are often used interchangeably and refer to the total pool of UA (dissociated and undissociated forms) because the ratio urate/UA in blood remains constant.

UA is normally found in the human body as a product of endogenous purine metabolism, being produced in kidneys, liver, intestine, vascular endothelium, and other muscle tissue. In addition, it also can have an exogenous origin, linked to the diet. The normal concentration of UA in human blood ranges from 1.5 to 6.0 mg/dl in women, and from 2.5 to 7.0 mg/dl in men. This difference has been linked to the uricosuric effect of oestrogens in women.

UA concentration in humans is generally close to its solubility limit of 6.8 mg/dL; once the saturation limit is reached, it precipitates as monosodium urate (MSU) crystals. Additionally, different factors such as pH, temperature, ionic strength and binding to macromolecules (proteins and mucopolysaccharides) may modify its solubility.

UA is the end oxidation product of purine metabolism in humans. Purines and pyrimidines are heterocyclic aromatic organic compounds (amines) that are found in all living things. They are composed of a nitrogenous base: adenine and guanine for purines, and cytosine, uracil and thymine for pyrimidines. These compounds play a key role in the transfer of genetic information, energy storage (ATP and GTP), cell signaling (CAMP and cGMP) and as enzyme cofactors (NADH, NADPH, coenzyme A). The cellular pool of purines and pyrimidines is tightly maintained via two 5 complementary pathways: salvage (which uses preformed nitrogenous bases from nucleotide degradation) and de novo synthesis by a functional protein complex (known as purinosome for purine biosynthesis) when there is an increased demand (cell growth or division).

The degradation of purines involves the sequential action of several enzymes that starting from nucleotides eventually produces UA. Most ingested nucleic acids are degraded by pancreatic and intestinal enzymes to nucleotides and, then, into nucleosides. If nucleosides are not absorbed, they are further degraded. Eventually, xanthine is the point of convergence for the metabolism of purines. Adenosine 5′-monophosphate (AMP) is converted into inosine by two different ways. The phosphate group is removed from AMP by the enzyme 5′-nucleotidase to form adenosine and, then, the enzyme adenosine deaminase removes an amino group to form inosine.

Alternatively, the enzyme AMP deaminase converts AMP into inosine 5′-monophosphate (IMP) by removing an amino group, and then a nucleotidase removes the phosphate group to convert IMP in inosine. Likewise, guanosine 5′-monophosphate (GMP) is converted to guanosine by a nucleotidase and, then, to guanine by purine nucleoside phosphorylase (PNP).

Guanine and inosine are converted to UA by xanthine oxidase (XO) in two consecutive reactions, although inosine should be first transformed in hypoxanthine by purine nucleotide phosphorylase. Then, both hypoxanthine and guanine are oxidized to xanthine which can be further oxidized to UA.

As used herein, the term “high cholesterol” refers to disorders that lead to or are manifested by aberrant levels of circulating lipids. Normal levels of lipids are reported in medical treatises known to those of skill in the art. At the present time, the recommended level of HDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

The term “high triglycerides”, as used herein refers to disorders that lead to or are manifested by aberrant levels of circulating tryglicerides. At the present time, the recommended levels of triglycerides is less than 150 milligrams per deciliter (mg/dL). Higher levels are considered 151-200 mg/dl (borderline high), 201-499 mg/dl (high) and 500 mg/dl or higher (very high).

The term “oxidative stress” as described herein refers to the condition of oxidative damage resulting when the critical balance between free radical generation and antioxidant defenses is unfavorable. Oxidative stress, arising as a result of an imbalance between free radical production and antioxidant defenses, is associated with damage to a wide range of molecular species including lipids, proteins, and nucleic acids. An excess of oxidative stress can lead to the oxidation of lipids and proteins, which is associated with changes in their structure and functions. Short-term oxidative stress may occur in tissues injured by trauma, infection, heat injury, hypertoxia, toxins, and excessive exercise. These injured tissues produce increased radical generating enzymes (e.g., xanthine oxidase, lipogenase, cyclooxygenase) activation of phagocytes, release of free iron, copper ions, or a disruption of the electron transport chains of oxidative phosphorylation, producing excess ROS. Oxidative stress has been implicated in the etiology of several degenerative diseases, such as stroke, Parkinson's disease, Alzheimer's disease, rheumatoid arthritis, diabetes mellitus, peptic ulcer, gene mutations and cancer, heart and blood disorders, and inflammatory diseases. Oxidative stress is now thought to make a significant contribution to all inflammatory diseases (arthritis, vasculitis, glomerulonephritis, lupus erythematous, adult respiratory diseases syndrome), ischemic diseases (heart diseases, stroke, intestinal ischema), hernochromatosis, acquired immunodeficiency syndrome, emphysema, organ transplantation, gastric ulcers, hypertension and preeclampsia, neurological disorder (Alzheimer's disease, Parkinson's disease, muscular dystrophy), alcoholism, smoking-related diseases, and many others.

Another aspect of the present invention refers to the isolated strain of the invention, the bacterial culture of the invention or the pharmaceutical composition of the invention for the use in the prevention and/or treatment of primary or secondary infertility, from here onwards the fertility use of the invention.

Alternatively, the invention relates to a method for preventing and/or treating primary or secondary infertility, which comprises administering the isolated strain of the invention, the bacterial culture of the invention or the pharmaceutical composition of the invention to a subject in need thereof.

The expression “infertility” in the context of the present description refers to a disease of the male or female reproductive system defined by the failure to achieve a pregnancy after 12 months or more of regular unprotected sexual intercourse. In the male reproductive system, infertility is most commonly caused by problems in the ejection of semen, absence or low levels of sperm, or abnormal shape (morphology) and movement (motility) of the sperm. In the female reproductive system, infertility may be caused by a range of abnormalities of the ovaries, uterus, fallopian tubes, and the endocrine system, among others. Infertility can be primary or secondary. “Primary infertility” is when a pregnancy has never been achieved by a person, and “secondary infertility” is when at least one prior pregnancy has been achieved.

Another aspect of the present invention refers to the isolated strain of the invention, the bacterial culture of the invention or the pharmaceutical composition of the invention for the use in increasing the rate of reproductive success in a patient who has suffered from recurrent pregnancy loss, from here onwards the recurrent pregnancy loss use of the invention.

Alternatively, the invention relates to a method for increasing the rate of reproductive success in a patient who has suffered from recurrent pregnancy loss, which comprises administering the isolated strain of the invention, the bacterial culture of the invention or the pharmaceutical composition of the invention to the patient in need thereof.

The term “recurrent pregnancy loss” as used herein refers to the failure of two or more clinically recognized pregnancies before 20-24 weeks of gestation and includes embryonic and fetal losses. Recurrent pregnancy loss can be due to different causes, including: anatomical causes, malformations in the uterus, fibroids, causes related to lifestyle or environment: smoking, drug use, excessive consumption of alcohol or caffeine, obesity, untreated diseases such as thyroid disease or diabetes, abnormalities of the immune system or the blood clotting system.

In a particular embodiment of the use in increasing the rate of reproductive success in a patient who has suffered from recurrent pregnancy loss, the recurrent pregnancy loss is unexplained. The term “recurrent unexplained pregnancy loss”, as used here, refers to a recurrent pregnancy loss for which, after performing a battery of diagnostic tests on both the woman and her male partner, no cause has been identify.

The expression “increasing the rate of reproductive success in a patient who has suffered from recurrent pregnancy loss” refers to the statistical significant rise of the probability of a patient suffering from recurrent pregnancy lost to achieve a successful pregnancy. The rate of miscarriage varies greatly between individuals and therefore, the increase in rate of reproductive success, as the expert will understand, will be different between individuals. The rate of miscarriage can be calculated as previously established (Magnus et al, BMJ 2019; 364:1869; Lund et al., Obstetrics & Gynecology: 2012-Volume 119-Issue 1-p 37-43). In a particular embodiment of the use in increasing the rate of reproductive success in a patient who has suffered from recurrent pregnancy loss, the increase in the rate will be of at least 0.1%, at least 0.2%, at least 0.4%, at least 0.6%, at least 0.8%, at least 1%, at least 10%, at least 20%, at least 30%, at least 40%, at least 50% of the rate of miscarriage in a patient.

In a particular embodiment of the infertility use of the invention or the recurrent pregnancy loss use of the invention, the administration of the isolated strain of the invention, the bacterial culture of the invention or the pharmaceutical composition of the invention is done prior to, after of or simultaneously with an assisted reproductive treatment. In a particular embodiment of the infertility use of the invention or the recurrent pregnancy loss use of the invention, the administration of the isolated strain of the invention, the bacterial culture of the invention or the pharmaceutical composition of the invention is done for at least 1 month, at least 2 months, at least 3 months, at least 4 months, at least 5 months, at least 6 months, at least 7 months, at least 8 months, at least 9 months, at least 10 months, at least 11 months, at least 1 year before receiving assisted reproductive treatment. In a particular embodiment of the infertility use of the invention or the recurrent pregnancy loss use of the invention, the administration of the isolated strain of the invention, the bacterial culture of the invention or the pharmaceutical composition of the invention is done is administered during the entire period of time that the female subject receives the assisted reproduction treatment.

The term “assisted reproduction treatment” or “medically assisted reproduction treatment” as used herein refers to a medical treatment intended to promote pregnancy in the case of infertility problems, whether male, female or both types, as well as in the case of women without a partner or with a female partner who want a pregnancy with donor sperm. Assisted reproduction is characterized by the stimulation or induction of ovulation and may include direct action on the gametes (oocytes and/or sperm) in order to promote fertilization and the transfer or deposit of embryos in the uterine cavity.

Examples of assisted reproductive treatments include, without limitation, ovulation induction, controlled ovarian stimulation, timed intercourse, intrauterine, intracervical and intravaginal artificial insemination, either with semen from the partner or a donor, in vitro fertilization (IVF), sperm microinjection (ICSI), sperm extraction, oocyte donation and fertility preservation.

In a particular embodiment of the infertility use of the invention or the recurrent pregnancy loss use of the invention, the administration of the isolated strain of the invention, the bacterial culture of the invention or the pharmaceutical composition of the invention is done is administered after the subject has received the assisted reproduction treatment for at least 1 week, at least 2 weeks, at least 3 weeks, at least 4 weeks or more.

In a particular embodiment of the infertility use of the invention or the recurrent pregnancy loss use of the invention, wherein the isolated strain, the bacterial culture or the pharmaceutical composition is administrated as a freeze-dried powder for at least 6 months or until pregnancy is achieved.

In another particular embodiment of the infertility use of the invention or the recurrent pregnancy loss use of the invention, once the pregnancy use achieve the administration of the isolated strain of the invention, the bacterial culture of the invention or the pharmaceutical composition of the invention is continued until at least week 1, at least week 2, at least week 3, at least week 4, at least week 5, at least week 6, at least week 7, at least week 8, at least week 9, at least week 10, at least week 11, at least week 12, at least week 13, at least week 14, at least week 15 of the pregnancy.

The isolated strain of the invention is useful not only in the treatment of infertility and recurrent pregnancy loss but can be also useful in the prevention of miscarriages. Therefore, another aspect of the present invention relates to the isolated strain of the invention, the bacterial culture of the invention or the pharmaceutical composition of the invention for use in the prevention of miscarriage of pregnancy in the first trimester of pregnancy, for here onwards the miscarriage use of the invention. The use refers to the administration prior to conception as well as to the administration during the first trimester of pregnancy, resultin in both cases in a reduced risk of miscarriage during the first three months of pregnancy.

Alternatively, the invention relates to a method for preventing miscarriage of pregnancy in the first trimester of pregnancy which comprises administering the isolated strain of the invention, the bacterial culture of the invention or the pharmaceutical composition of the invention to a subject in need thereof.

In a particular embodiment of the miscarriage use of the invention the isolated strain of the invention, the bacterial culture of the invention or the pharmaceutical composition of the invention is administrated for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least week 15 after pregnancy.

The miscarriage use of the invention, as the expert will understand can be preceded by a treatment before pregnancy is achieved in order to increase the rate of pregnancy or to treat infertility. Therefore, in a particular embodiment of the miscarriage use of the invention, the treatment is preceded by the infertility use of the invention or the recurrent pregnancy loss use of the invention.

The term “pregnancy week” refers to the time elapsed since the date of the last menstruation, assuming that the menstrual cycle lasts 28 days and that fertilization has occurred on day 14 of said cycle.

In another particular embodiment of the miscarriage use of the invention the isolated strain of the invention, the bacterial culture of the invention or the pharmaceutical composition of the invention is administrated in the first 15 weeks of pregnancy. In a particular embodiment of the infertility use of the invention, the recurrent pregnancy loss use of the invention or the miscarriage use of the invention, the isolated strain of the invention, the bacterial culture of the invention or the pharmaceutical composition of the invention is administrated to a subject with infertility or recurrent pregnancy loss in combination with an assisted reproductive treatment.

The isolated strain of the invention may be dosed according to the requirements of the treatment and under medical guidance as to obtain the desired outcome. In a particular embodiment of the infertility use of the invention, the recurrent pregnancy loss use of the invention or the miscarriage use of the invention the isolated strain, the bacterial culture or the pharmaceutical composition is administrated at least 1 time, at least 2 times, at least 3 times, at least 4 times, at least 5 times, at least 6 times, at least 7 times, at least 8 times, at least 9 times, at least 10 times, at least 11 times, at least 12 times a day. In another particular embodiment of the infertility use of the invention, the recurrent pregnancy loss use of the invention or the miscarriage use of the invention the isolated strain, the bacterial culture or the pharmaceutical composition is administrated at least at least 1 times, at least 2 times, at least 3 times, at least 4 times, at least 5 times, at least 6 times, at least 7 times a week. In another particular embodiment of the infertility use of the invention, the recurrent pregnancy loss use of the invention or the miscarriage use of the invention, the isolated strain, the bacterial culture or the pharmaceutical composition is administrated at least once a day, at least once a week, at least once a month.

The dosing of the isolated strain of the invention can combine several regimes of administration, having interval periods wherein no dose is administrated between periods of administration.

Together with the dosing the isolated strain of the invention, the bacterial culture of the invention of the pharmaceutical composition of the invention can be formulated in several forms depending on the desired use. In a particular embodiment of the infertility use of the invention, the recurrent pregnancy loss use of the invention or the miscarriage use of the invention, the isolated strain, the bacterial culture or the pharmaceutical composition is formulated for oral, vaginal, rectal, anal, intramammary, intravenous, intra-arterial, parenteral, intracranial, topical, ocular, respiratory or nasal administration, preferably oral.

In a particular embodiment of the infertility use of the invention, the recurrent pregnancy loss use of the invention or the miscarriage use of the invention, the isolated strain, the bacterial culture or the pharmaceutical composition administrated comprises from about 8 log 10 CFU to about 10 log 10 CFU of the isolated strain of the invention, preferably 9 log 10 CFU. In another more particular embodiment the infertility use of the invention, the recurrent pregnancy loss use of the invention or the miscarriage use of the invention, the isolated strain, the bacterial culture or the pharmaceutical composition is administrated at least once a day and comprises around 9 log 10 CFU.

### Other Products of the Invention

All the terms, definitions, and embodiments previous disclosed in relation to previous aspects are equally valid for the following aspects and their embodiments.

Due to the characteristics of the isolated strain of the invention, it has the properties of a probiotic. Another aspect of the present invention is the use of the isolated strain of the invention or the bacterial culture of the invention as a probiotic.

The term “probiotic” as used herein refers to live microorganisms which when administered in adequate amounts confer a health benefit on the host.

The isolated strain of the invention of the bacterial culture of the invention find further uses in the manufacture of edible products and medicinal devices. An aspect of the present invention relates to a food product comprising the isolated strain of the invention of the bacterial culture of the invention and an appropriate amount of an edible ingredient, from here onwards the food product of the invention.

The term “food product” as used herein includes both solid and liquid ingestible materials for man or animals, which materials usually do, but need not, have nutritional value. Thus, food products includes meats, gravies, soups, convenience foods, malt, alcoholic, and other beverages, milk and dairy products, seafoods including fish, crustaceans and mollusks, candies, vegetables, cereals, soft drinks, snacks, dog and cat foods, other veterinary products.

Within the context of the present invention, the term “edible ingredient” means any kind of edible ingredient or mixture of ingredients that may be safely consumed by a human or animal.

In a particular embodiment of the food product of the invention is selected from the group consisting of a milk product, a yogurt, a curd, a cheese, a fermented milk, a powdered milk, a milk based fermented product, a meat based fermented product, an ice-cream, a cereal based fermented product, a beverage, a snack, a flour, a chewing-gum, a sweet, a sweet food, a pet food, a dietary or food supplement, a functional food, a clinical nutrition formula, a nutritional complement, a formula for pregnant woman, a formula for the elderly and an infant formula.

Another aspect of the present invention relates to a nutraceutical comprising the isolated strain of the invention or the bacterial culture of the invention.

Another aspect of the present invention relates to a nutraceutical composition comprising the isolated strain of the invention or the bacterial culture of the invention and a food acceptable adjuvant and/or food ingredient.

In the present invention, the term “nutraceutical” is understood to refer to a food that has a beneficial effect on health. Similarly, the term “nutraceutical” may be applied to chemical extracts or compounds derived from common foods. Examples of foods to which nutraceutical properties are attributed include olive oil, red wine, broccoli and soya, etc. Nutraceuticals are usually employed in nutritional mixtures and in the pharmaceutical industry. Like various food products, some nutritional supplements may also be classified as nutraceuticals, for example fatty acids such as omega-3 derived from fish oils and from some vegetables or antioxidants and vitamins.

The expression “food acceptable adjuvant and/or food ingredient” according to the present invention refers to protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

Moreover, a multi-vitamin and mineral supplement may be added to nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions according to the present invention may be in any galenic form that is suitable for administering to the body, especially in any form that is conventional for oral administration, e.g. in solid forms such as (additives/supplements for) food or feed, food or feed premix, fortified food or feed, tablets, pills, granules, dragées, capsules and effervescent formulations, such as powders and tablets, or in liquid forms, such as solutions, emulsions or suspensions as e.g. beverages, pastes and oily suspensions. The pastes may be incorporated in hard or soft shell capsules, whereby the capsules feature e.g. a matrix of (fish, swine, poultry, cow) gelatine, plant proteins or ligninsulfonate. Examples for other application forms are those for transdermal, parenteral or injectable administration. The dietary and pharmaceutical compositions may be in the form of controlled (delayed) release formulations.

Another aspect of the present invention relates to a medical device comprising an effective amount of the isolated strain of the invention or the bacterial culture of the invention as an active ingredient.

The term “medical device” refers to, as defined by the European Directive 2007/47/EC, any product, instrument, device, apparatus, computer program, material or article used in the medico sanitary field and being regulated by the European Directive 90/385/CEE of active implantable medical device, 98/79/CE of medical devices for in vitro diagnostic, and 93/42/CEE for general medical devices. The medical devices are included in the sanitary technologies. They are used in human beings for the diagnostic, prevention, control, treatment or alleviation of a disease, for the compensation of a deficiency; for investigating, substituting or modification of anatomy or a physiologic process; and for the regulation of contraception. Medical devices of the present invention relate to compositions or formulations (“medical device compositions”), or to products or devices comprising said compositions or formulations, comprising an effective amount of the isolates strain of the invention (L. salivarius CECT 30632 or a variant thereof and/or L. salivarius CECT 30641 or a variant thereof) as active ingredient. The term “medical device composition” is used herein to identify the compositions according to the invention, which compositions, as such (by their selves) are catalogued by the competent sanitary authorities as medical devices.

The medical device of the present invention allows the application or administration of the strain of the invention to a desired tissue surface, for example, in order to pharmaceutically and/or via the medical device itself treat or take care of the surface to which it is applied. In some embodiments, the composition comprising the strain of the invention or any mutant or variant thereof is per se catalogued by the authorities as a medical device. In other particular embodiments of the medical devices, they correspond to sanitary articles that include woven or non-woven fabrics, in which the strain of the invention is homogeneously distributed along the surface of the fabric. This distribution can be performed, optionally, with the aid of a solvent. The solvent may also optionally be dried after layer or coating of the composition comprising the effective amount of the strain, is totally or partially distributed onto the surface of the fabric, thus obtaining fabrics with the properties conferred by the strain according to the invention.

### Non-Medical Uses of the Invention

All the terms, definitions, and embodiments previous disclosed in relation to previous aspects are equally valid for the following aspects and their embodiments.

Further to the medical uses previously described above, the isolated strain of the invention and/or the bacterial culture of the invention have further non-therapeutic uses. The term “non-therapeutic method”, as uses herein, refers to a process, action, application or the like on a subject that:
(i) is not directed to the improvement of a condition or disease, directly or indirectly, or to slowing the progression of a condition or disease, or to ameliorating one or more symptoms of a condition or disease of said subject, and
(ii) is not directed to delay the onset of the disorder or disease, or prevent the individual from developing the disorder or disease at all.

As such, another aspect of the present invention relates to a non-therapeutic method for the regulation of the epithelial mucosa pH in a subject, from here onwards the non-therapeutic method I of the invention, comprising the administration of the isolated strain of the invention, the bacterial culture according to the invention, the food product of the invention or the nutraceutical of the invention to the subject.

The term “epithelial mucosa” as used herein refers to the barrier that separates the underlying tissues from their environment. It consists of two layers, the surface stratified squamous epithelium and the deeper lamina propria. In keratinized oral mucosa, the epithelium is composed of the four layers stratum basale, stratum spinosum, stratum granulosum, and stratum corneum. In non-keratinised epithelium, the stratum basale is followed by the stratum filamentosum and the stratum distendum. In a particular embodiment of the non-therapeutic method I of the invention the epithelial mucosa is the vaginal epithelial mucosa or the intestinal epithelial mucosa.

The expression “regulation of the epithelium mucosa pH” refers to a process, procedure or technique to bring the pH of the epithelium mucosa to a pH, which is closer to the pH considered healthy for an individual of identical characteristics of the subject being treated. For example, the pH range of a cervical/vaginal epithelium mucosa considered healthy is between 3.5 and 5.0, depending on the age of the subject as well as the moment of the menstrual cycle. In another example, the pH of the intestinal epithelium mucosa varies depending on the region of the intestinal track, ranging from about 5.5 to about 7.5.

In a particular embodiment of the non-therapeutic method I of the invention the pH of the epithelium mucosa varies by at least 0.1%, at least 0.5%, at least 1%, at least 2%, at least 3%, at least 4%, at least 5%, at least 6%, at least 7%, at least 8%, at least 9%, at least 10% towards the average value of the range considered healthy for the subject.

Another aspect of the present invention relates to a non-therapeutic method for the regulation of the cervicovaginal microbiota in a subject, from here onwards the non-therapeutic method II of the invention, comprising the administration of the isolated strain of the invention, the bacterial culture of the invention, the food product of the invention or the nutraceutical of the invention.

The term “cervicovaginal microbiota” as used herein refers to the microorganisms which colonize the cervicovaginal lumen. The cervicovaginal microbiota is composed of hundreds of bacteria and a smaller quantity of fungi, which live in the cervicovaginal lumen.

The expression “regulation of the cervicovaginal microbiota” refers to the alteration of the type, number and ratio of species of bacteria/fungi present in the microbiota, wherein the alteration can be the appearance of previous not presence or absent species, the increase/reduction of the of total number of individuals of a species, the increase/reduction of the number of individuals of a species in relation to another species or the total number of species present in the microbiota.

Another aspect of the present invention relates to a non-therapeutic method for promoting an increase in the content of vaginal Lactobacillus species in a subject comprising the administration of the isolated strain of the invention, the bacterial culture of the invention, the food product of the invention or the nutraceutical of the invention.

The term “Lactobacillus species” as used herein refers to Gram-positive, aerotolerant anaerobes or microaerophilic, rod-shaped, non-spore-forming bacteria species, which are normal microbiota of the human mouth, gastrointestinal tract, and female genital tract, where they produce lactic acid (resulting in a low pH) and competitively inhibit pathogenic organisms. The term “Lactobacillus” is commonly applied to any genus derived from the reclassification of the former genus Lactobacillus in the year 2020 (Zheng J, Wittouck S, Salvetti E, Franz CMAP, Harris H M B, Mattarelli P, O'Toole P W, Pot B, Vandamme P, Walter J, Watanabe K, Wuyts S, Felis G E, Ganzle M G, Lebeer S. A taxonomic note on the genus Lactobacillus: Description of 23 novel genera, emended description of the genus Lactobacillus Beijerinck 1901, and union of Lactobacilliae and Leuconostocaceae. Int J Syst Evol Microbiol. 2020 April; 70(4):2782-2858. doi: 10.1099/ijsem.0.004107). Examples of Lactobacillus species are Lactobacillus acetotolerans, Lactobacillus acidophilus, Limolactobacillus alvi, Lactobacillus amylolyticus, Lactobacillus amylovorus, Lactobacillus apis, Loigoactobacillus backii, Lactobacillus bombicola, Lacticaseibacillus casei, Lactobacillus colini, Lactobacillus crispatus, Lactobacillus delbrueckii, Lactobacillus equicursoris, Limosilactobacillus fermentum, Lactobacillus fornicalis, Lactobacillus gallinarum, Lactobacillus gasseri, Lactobacillus gigeriorum, Companilactobacillus ginsenosidimutans, Lactobacillus hamster, Lactobacillus helsingborgensis, Lactobacillus helveticus, Lactobacillus hominis, Lactobacillus iners, Lactobacillus intestinalis, Lactobacillus jensenii, Acetilactobacillus jinshani, Lactobacillus johnsonii, Lactobacillus kalixensis, Lactobacillus kefiranofaciens, Lactobacillus kimbladii, Lactobacillus kitasatonis, Lactobacillus kullabergensis, Lactobacillus melliventris, Lactobacillus mulieris, Lactobacillus nasalidis, Lactobacillus panisapium, Lactobacillus paragasseri, Lactobacillus pasteurii, Lactiplantibacillus plantarum, Lactobacillus porci, Lactobacillus psittaci, Lentilactobacillus raoultii, Limosilactobacillus reuteri, Lacticaseibacillus rhamnosus, Lactobacillus rodentium, Lactobacillus rogosae, Lactobacillus taiwanensis, Limosilactobacillus salivarius, Lactobacillus timonensis, Lactobacillus ultunensis, Lactobacillus xujianguonis.

In a particular embodiment of the non-therapeutic method II of the invention the species of Lactobacillus increased are selected from a group consisting of: L. iners, L. crispatus, L. gasseri, L. jensenii, L. fermentum, L. salivarius and any combination thereof.

The expression “promoting an increase in the content of vaginal Lactobacillus species” as used herein refers to the process of increasing the number of Lactobacillus species, through the appearance of new lactobacillus species, which were absent, or the reappearance of Lactobacillus species previously present.

Another aspect of the present invention relates to a non-therapeutic method for promoting an increase in soluble immune factors in the internal mucosal of a subject, from here onwards the non-therapeutic method Ill of the invention, comprising the administration of the isolated strain of the invention, the bacterial culture of the invention, the food product of the invention or the nutraceutical of the invention to the subject.

The term “soluble immune factors” as used herein refers to a diverse group of molecules, which are present in the blood and which have a substantial impact on both the innate and adaptive immune responses. Examples of such molecules are the complement system, antibodies, and anti-microbial proteins and peptides can directly interact with potential pathogens, protecting against systemic infection.

The expression “promoting an increase in soluble immune factors” as used herein, refers to a method or process of leading to an increase in the levels of the soluble immune factors, i.e., the amount of the soluble immune factors present in the internal mucosa of the subject, wherein said levels are increase in a sample of said internal mucosa after having been administrated the isolated strain of the invention, in comparison with a sample of the mucosa before having been administrated the isolated strain of the invention. In a particular embodiment of the method III of the invention, the soluble immune factors are protein immune factors.

The term “increase in the level with respect to the reference value” indicates that the level of the soluble immune factor is higher than its reference value. The level a solule immune factor is considered to be higher than its reference value when it is higher by at least 1.5%, at least 2%, at least 5%, at least 10%, at least 15%, at least 20%, at least, 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 200% or higher with respect to the reference value. The level of a soluble immune factor can be determined by any method known in the field for the determination and quantification of a protein in a sample. By way of non-limiting illustration, the level of a protein can be determined by a technique that comprises the use of antibodies with the ability to specifically bind to the analyzed protein (or to fragments of the same that contain the antigenic determinants) and subsequently the quantification of the resulting antigen-antibody complexes, or alternatively by a technique that does not comprise the use of antibodies such as, for example, by techniques based on mass spectroscopy. Antibodies can be monoclonal, polyclonal, or fragments thereof, Fv, Fab, Fab′ and F(ab′)₂, scFv, diabodies, tribodies, tetrabodies, and humanized antibodies. Similarly, antibodies can be labeled. Illustrative, non-limiting examples of labels that may be used include radioactive isotopes, enzymes, fluorophores, chemiluminescent reagents, enzyme cofactors or substrates, enzyme inhibitors, particles or dyes. There is a wide variety of known tests that can be used in accordance with the present invention, such as the combined application of unlabeled antibodies (primary antibodies) and labeled antibodies (secondary antibodies), Western blotting or immunoblotting, ELISA (enzyme-linked immunosorbent assay), RIA (radioimmunoassay), competitive EIA (enzyme immunoassay), DAS-ELISA (double antibody sandwich ELISA), two-dimensional gel electrophoresis, capillary electrophoresis, immunocytochemical and immunohistochemical techniques, immunoturbidimetry, immunofluorescence, techniques based on the use of biochips protein microarrays, including specific antibodies or colloidal precipitation-based assays in formats such as test strips and antibody-bound quantum dot-based assays. Other ways of detecting and quantifying proteins include, for example, affinity chromatography techniques or ligand binding assays.

In a particular embodiment, the level of the soluble immune factor is determined by a multiplex immunoassay. In another particular embodiment, the level of soluble immune factor is determined by ELISA.

In a particular embodiment of the method Ill of the invention, the soluble immune factors are selected from a group consisting of TGF-β1, TGF-β2, VEGF and any combination thereof.

The term “VEGF” or “VEGF-A” or “Vascular Endothelial Growth Factor” refers to a member of the PDGF/VEGF family of growth factors. It is a heparin-binding protein, which exists as a homodimer. disulfide-linked. This growth factor induces the proliferation and migration of vascular endothelial cells, and is essential for physiological and pathological angiogenesis. In humans, VEGF corresponds to the protein defined by accession number P15692 in the database. UniProtKB/Swiss-Prot (entry version 257, Feb. 23, 2022; sequence version 2, Nov. 16, 2001).

The term TGF-β1 “or” transforming growth factor beta 1 “refers to a protein belonging to the superfamily of transforming growth factors beta of cytokines. It is a secretory protein involved in functions such as control of cell growth, cell proliferation, differentiation processes and apoptosis. In humans, TGF-β1 corresponds to the protein defined by accession number P01137 in the UniProtKB/Swiss-Prot database (entry version 262, Feb. 23, 2022; sequence version 2, Feb. 1, 1991).

The term “TGF-β2” or “transforming growth factor beta 2” refers to a protein belonging to the superfamily of transforming growth factors beta of cytokines. It is a protein involved in angiogenesis and heart development functions. In humans, TGF-β2 corresponds to the protein defined by accession number P61812 in the UniProtKB/Swiss-Prot database (entry version 190, Feb. 23, 2022; sequence version 1, Aug. 1 1988).

Another aspect of the present invention relates to a non-therapeutic method for improving the mental well-being of a subject, from here onwards the method IV of the invention, comprising the administration of the isolated strain of the invention, the bacterial culture of the invention, the food product of the invention or the nutraceutical of the invention to the subject.

In the context of the present description the term “mental well-being” or “mental health”, as defined by the World Health Organization, refers to a state of well-being in which the individual realizes his or her own abilities, can cope with the normal stresses of life, can work productively and fruitfully, and is able to make a contribution to his or her community.

The expression “improving the mental the wellbeing” refers to the process of increasing, stopping or reducing the decrease of the mental wellbeing of the subject. The measurement of the mental wellbeing can be accomplished by the use of several tests, such as, without limitation, the twenty-item PANAS scale (Whatson et al., J Pers Soc Psych 1988, 6(54):1063-70), the five-item Satisfaction With Life Scale (SWLS) (Diener et al., J Pers Assess 1985, 49(1):71-75), the 54 item Scale of Psychological Well-Being (SPWB) (Ryff and Keyes, J Pers Soc Psychol 1995, 69:719-727) or the positively worded five item WHO Wellbeing Index (WHO-5) (Bech, QoL Newsletter 2004, 32:15-16). In a particular embodiment of the method IV of the invention the mental well-being of the subject is measured by the five item World Health Organization Wellbeing Index. In another particular embodiment of the method IV of the invention the mental wellbeing of the subject is improved by an increase in the WHO-5 scale of at least 5, at least 10, at least 15, at least 20, at least 25, at least 30, at least 40, at least 50, at least 60 points, wherein the scale if form 0 to 100 points.

Another aspect of the present invention relates to a non-therapeutic method for regulating the circulating estrogen levels in a subject comprising the administration of the isolated strain of the invention, the bacterial culture of the invention, the food product of the invention or the nutraceutical of the invention to the subject.

“Regulating”, as used herein relates to the need of increasing or reducing the levels of a particular substance in view of its abnormal levels.

In another aspect, the invention relates to a non-therapeutic method for increasing the levels of circulating riboflavin in a subject comprising the administration of the isolated strain of the invention, the bacterial culture of the invention, edible product of the invention or the nutraceutical of the invention to the subject.

The expression “riboflavin”, as used herein relates to vitamin B, water-soluble vitamin that the body uses to metabolize carbohydrates, fats, and protein into glucose for energy and which corresponds to the compound having the CAS number 83-88-5.

In another aspect, the invention relates to a non-therapeutic method for reducing the levels of circulating uric acid in a subject comprising the administration of the isolated strain of the invention, the bacterial culture of the invention, edible product of the invention or the nutraceutical of the invention to the subject.

In another aspect, the invention relates to a non-therapeutic method for increasing the performance of activities of daily living in a subject comprising the administration of the isolated strain of the invention, the bacterial culture of the invention, edible product of the invention or the nutraceutical of the invention to the subject.

In another aspect, the invention relates to a non-therapeutic method for increasing the nutritional state of a subject comprising the administration of the isolated strain of the invention, the bacterial culture of the invention, edible product of the invention or the nutraceutical of the invention to the subject.

“Nutritional state or status”, as used herein relates to the condition of the body in those respects influenced by the diet; the levels of nutrients in the body and the ability of those levels to maintain normal metabolic integrity. For adults, general adequacy is assessed by measuring weight and height; the result is commonly expressed as the body mass index, the ratio of weight (kg) to height² (m). Body fat may also be estimated, by measuring skinfold thickness, and muscle diameter is also measured. For children, weight and height for age are compared with standard data for adequately nourished children. The increase in the circumference of the head and the development of bones may also be measured. Status with respect to individual vitamins and minerals is normally determined by laboratory tests, either measuring the blood and urine concentrations of the nutrients and their metabolites, or by testing for specific metabolic responses.

In another aspect, the invention relates to a non-therapeutic method for regulating the circulating levels of total cholesterol of a subject comprising the administration of the isolated strain of the invention, the bacterial culture of the invention, edible product of the invention or the nutraceutical of the invention to a subject in need thereof. In another aspect, the invention relates to a non-therapeutic method for regulating the circulating levels of triglycerides of a subject comprising the administration of the isolated strain of the invention, the bacterial culture of the invention, edible product of the invention or the nutraceutical of the invention to a subject in need thereof.

In another aspect, the invention relates to a cosmetic method for reducing vaginal discharge which comprises the administration of the isolated strain of the invention, the bacterial culture of the invention, edible product of the invention or the nutraceutical of the invention to a subject in need thereof.

The invention will be described by way of the following examples, which are to be considered as merely illustrative and not limitative of the scope of the invention.

### EXAMPLES

### Materials and Methods

### 1. PHASE 1: Preliminary Analysis for the Selection of Lactic Acid Bacteria Strains

### 1.1. Kinetic Parameter Assays

To determine the starting strain growth kinetics, pure cultures were obtained from the frozen stock after 24 h of incubation in MRS (Man, Rogosa, and Sharpe) medium (OXOID) at 37° C. or 32° C. (the latter temperature was used in the case of Lactococcus spp. strains). The appropriate 10-fold dilutions which were used for inoculating 96-well microplates (300μl/well of each dilution) were performed based on these cultures. The microplates were incubated in automated equipment (Bioscreen C MBR, Oy Growth Curves Ab Ltd, Finland) which measures absorbance at 600 nm (A₆₀₀) at regular time intervals, in an environment with controlled temperature and relative humidity. Based on the growth curves obtained (A₆₀₀ VS. time), the following were calculated: 1) specific maximum growth rate (μₘₐₓ) (h⁻¹) and 2) generation time (g) (h). At the same time, microbial counts of the maximum cell population reached after 24 h of incubation were carried out. To that end, 10-fold dilutions of the cultures were seeded in plates containing MRS which were incubated for 24 h at 37° C. or 32° C. The cell population was expressed as log colony forming units (CFU) per mL (log₁₀ CFU/mL). Based on these parameters, the strains were classified into 4 categories: slow growth (μₘₐₓ<1 h⁻¹), moderate growth (μₘₐₓ=1-1.2 h⁻¹), rapid growth (μₘₐₓ=1.1-1.5 h⁻¹), and very rapid growth (μₘₐₓ>1.5 h⁻¹). Slow and moderate growth strains were excluded in this step.

### 1.2. Antibiotic Sensitivity Assays

To determine if any of the selected strains were resistant to the most widely used antibiotics, an assay was carried out using Etest® strips (BioMérieux). These paper strips are impregnated with increasing concentrations of antibiotics (ampicillin, vancomycin, gentamicin, kanamycin, streptomycin, erythromycin, clindamycin, tetracycline, and chloramphenicol). For the assays, 100 μl of 24-h cultures were seeded in depth in MRS. Once the agar solidified, six strips of each of the assayed antibiotics were placed on the surface of the plates. After 24 h of incubation, the Minimum Inhibitory Concentration (MIC) in μg/ml, which is the minimum antibiotic concentration with inhibitory effect against the evaluated microorganism, was determined. To that end, a horizontal line is drawn on the E-test strip which coincides with the appearance of an inhibition halo.

To determine whether the strain was resistant (R) or sensitive(S) to each of the evaluated antibiotics, experimental MICs were compared with the reference values published by the European Food Safety Authority (EFSA, 2018 “Guidance on microorganisms used as feed additives or as production organisms”).

### 2. PHASE 2: Determination of β-Glucosidase and β-Glucuronidase Activities of the Selected Strains

### 2.1. Obtaining Cell-Free Supernatants and Cell Extracts

Extra- and intracellular enzymatic activities were determined according to the protocol described in Dabek et al. (2008). Cell-free supernatants (CFSs) were obtained after centrifuging (5,000 rpm/4° C./15 min) the 24-h cultures in MRS (incubated at 37° C. or 32° C., in the case of lactococci). The CFSs were filtered through acetate filters (0.22 μm) and transferred to 15 mL FALCON tubes for storage at −20° C. To obtain cell extracts, the cells were washed 2 times with 3 mL of 100 mM phosphate buffer (PBS) at pH 6 and suspended in 1 mL of 100 mM PBS at pH 6. This volume was transferred to a microtube with a screw cap containing glass microspheres ($=106 nm). For cell rupture, the microtubes were introduced in the FAST-PREP-FP120 equipment with the following cycle: 30 s at speed 6 (5,000 rpm)/30 s of cooling on ice/30 s at speed 6 (5,000 rpm). The samples were then centrifuged (13,000 rpm/4° C./10 min) and the cell extract was transferred to an Eppendorf tube for storage at −20° C.

### 2.2. Determination of β-Glucosidase and β-Glucuronidase Activities

As substrates for enzymatic reactions, 0.2 and 1.0 mM solutions in 100 mM PBS (pH 6.0) of 4-nitrophenyl-β-D-glucopyranoside (β-glucosidase substrate) and 4-nitrophenyl-β-D-glucuronide (β-glucuronidase substrate) were used. 75 μL of each of the samples (CFSs or cell extracts of each strain) and 65 μl of the substrate solutions were added in a multiwell plate. Both the samples and the substrates were tempered previously at 37° C. After 30 minutes of incubation at the same temperature, the content of 4-nitrophenol (reaction product) released into the medium was determined spectrophotometrically at a wavelength of 405 nm using a multiwell plate reader (Labsystems IEMS Reader M F; Genesis Software). The assays were carried out in triplicate. At the same time, a calibration curve was made using known concentrations of 4-nitrophenol in order to calculate the concentration of the product of the enzymatic reaction. Those strains of interest with different patterns of enzymatic activities were selected for phase 3.

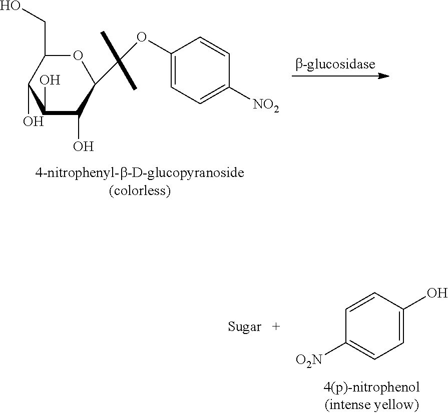

Enzymatic reaction scheme in the assay. The line indicates the bond on which the enzyme acts.

### 3. PHASE 3: Determination of Estrogen Metabolic Activity of the Strains Selected in Phase 2

### 3.1. Obtaining the Samples

To evaluate the capacity of the selected strains to metabolize estrogens, solutions of three primary estrogens [estrone (EM1), estriol (EM2), and 17β-estradiol (EM3)] in ethanol (HPLC purity) were first prepared at a concentration of 0.1 M. Then, 10 UL of said solutions were added to 10 mL of MRS, obtaining a final concentration of primary estrogens in the culture medium of between 26 and 30 ppm (μg/mL) [specifically, (EM1) 26 ppm, (EM2) 28 ppm, and (EM3) 30 ppm]. These media with estrogens were inoculated with the selected strains and incubated for 48 h at 37° C. After incubation, the cultures were centrifuged at 5,000 rpm/4° C./20 minutes, separating the cell pellet from the supernatant. This last fraction was filtered by means of an acetate filter (0.22 μm) and kept at −32° C. until use. At the same time, growth controls were performed in media without estrogens and in media with the same concentration of ethanol used for dissolving these compounds. It was confirmed that ethanol at the concentration used did not show any inhibitory effect on the cells.

### 3.2. Extraction of Estrogens

The extraction of free estrogens (FEs) and total estrogens (TEs) (FEs and conjugates) from frozen extracts was carried out following the protocol described in Xu et al. (2007), with some modifications. For the extraction of FEs, 0.5 mL of the extracts obtained as described in the preceding section were mixed with 8 mL of dichloromethane (Sigma Aldrich, HPLC quality) and subjected to a slow reverse extraction process (turning the tubes over) 360° for 30 minutes at 50 rpm. Slow extraction preserves the highly oxidizable lipid fractions, preventing the generation of air bubbles and damage on these compounds. The aqueous fraction was discarded and to recover the organic fraction the solvent was first evaporated using a nitrogen stream. For the extraction of TEs, the same method was used, including in this case a prior step of enzymatic hydrolysis to release conjugated estrogens (CEs). In this step, 0.5 mL of enzymatic hydrolysis buffer containing 2 mg of L-ascorbic acid, 5 μL of β-glucuronidase/sulfatase (Sigma Aldrich), and 0.5 mL of 0.15 M acetate buffer, pH 4.1, were added to 0.5 mL of extract. The reaction mixture was incubated at 37° C. for 20 h. After this step, the extraction method described above was followed. At the same time, the extraction of FEs and TEs in the control samples (estrogen-free MRS medium and cell-free MRS medium with estrogens) was carried out.

### 3.3. Determination of Estrogens and Derivatives by Means of HPLC/MS

This determination was performed according to the methodology described in Xu et al. (2007). The commercial standards of primary estrogens and their estrogenic derivatives [estrone, estriol, 17β-estradiol, 2-hydroxy-estrone-3-methyl-ether, 2-methoxy-estradiol, 4-methoxy-17β-estradiol, 4-methoxy-estrone, 16α-hydroxy-estrone, 2-hydroxy-estrone, 4-hydroxy-estrone, 17-epi-estriol, 2-methoxy-estrone] were reconstituted in 100 μL of methanol, the final concentration achieved being 1 ppm. The assays were performed in the Spectrophotometry Center at UCM (Complutense University of Madrid). The conditions of analysis were the following: the equipment used was LC-ESI-QQQ (LCMS-8030, Shimadzu) with a Poroshell 120 PhenylHexyl, 2.1×50 mm, 2.7 μm column. Phase A: H₂O+0.1% formic acid, and Phase B: Methanol+0.1% formic acid. For the HPLC run, the following parameters were used (percentage of each mobile phase and start time): 5% phase B, 1 min; 40% phase B, 11 min; 64% phase B, 12 min; 95% phase B, 13 min; 95% phase B, 14 min; and 5% phase B, final. The injected volume of reconstituted sample was 10 L. The flow rate used was 0.5 mL/minute and the total run time was 17 minutes. For selective quantification, the dynamic MRM mode (MRM transitions) which improves resolution sensitivity for compounds with a similar retention time, such as estrogen derivatives, was used.

### 3.4 Quantitative Determination of the Products Resulting from the Metabolism of the Strains on the Conjugated Estrogens

For the cell-free supernatants (SLC) of the cultures of the strains ES27 and ES43 obtained by adding exogenous conjugated substrates were collected. These SLC were treated with a mixture of organic solvents for the extraction of the lipid phase and their subsequent evaporation under a stream of nitrogen. The resulting organic phase was sent to the mass spectrometry service for analysis by LC-QQQ-MS (Shimadzu LCMS-8030 Triple cuadrupolo) in order to quantify the conjugated and free estrogens present in the samples. As substrates for the incubation of the bacteria, the conjugated commercial substrates of estrone (Estrone-3 β-glucuronide, E1-3G) and estradiol (Estradiol-3-R-D-glucuronide, E2-3G), were used. In parallel, the control samples corresponding to: culture medium without estrogens, culture medium samples with estrogens but without the microorganisms and ordinary cultures of the strains under study were processed in the same way. All assays were carried out in triplicate.

### Example 1. Isolation, Initial Selection and Identification of Ligilactobacillus salivarius MP98

Isolate MP98, deposited at the “Colección Española de Cultivos Tipo (CECT)” under the accession number CECT 30632, was isolated from the vaginal exudate of a normoweight lactating woman who met the following criteria: (a) no history of genitourinary tract infections and a low record of antibiotic use; (b) two previous term pregnancies without any complication; (c) abundant presence of lactobacilli in the vaginal sample (>6 log₁₀ colony-forming units [cfu]/swab) after culturing on MRS plates; (d) absence of detection of chlamydias, trichomonas, Gardnella vaginalis, Ureaplasma spp., S. agalactiae, Mycoplasma spp, and Candida spp. (or any other yeast) in the vaginal samples; and (e) negative (blood screening) to human immunodeficiency viruses (HIV), cytomegalovirus, human papillomavirus (HPV), gonorrhea and syphilis. In addition, strain-related criteria used to select L. salivarius MP98 were its qualified presumption of safety (QPS) status conceded by the European Food safety Authority (EFSA), and its ability to grow in MRS broth under aerobic conditions (@1×10⁸ cfu/mL after 16 h at 37° C.). The same strain was also isolated from feces of her healthy breastfed infant (initially the fecal isolate was called MP101 but genotyping of MP98 and MP101 revealed that actually both were the same strain).

The strain was identified as Lactobacillus salivarius, a species recently reclassified as Lagilactobacillus salivarius, by Matrix Assisted Laser Desorption Ionization-Time of Flight (MALDI-TOF) mass spectrometry (Bruker, Germany). In addition, it was also identified as L. salivarius by 16S ribosomal RNA (rRNA) gene partial sequencing performed using the primers and PCR conditions described by Kullen et al. [1] and shown below (SEQ ID NO:1).

GACAGTTACTCTCACTCGTGTTCTTCTCTAACAACAGAGTTTTACGATCCGAAGACCTTCTTCACTCACG
CGGCGTTGCTCCATCAGACTTGCGTCCATTGTGGAAGATTCCCTACTGCTGCCTCCCGTAGGAGTTTGGG
CCGTGTCTCAGTCCCAATGTGGCCGATCAACCTCTCAGTTCGGCTACGTATCATCACCTTGGTAGGCCGT
TACCCCACCAACTAGTTAATACGCCGCGGGTCCATCTAAAAGCGATAGCAGAACCATCTTTCATCTAAGG
ATCATGCGATCCTTAGAGATATACGGTATTAGCACCTGTTTCCAAGTGTTATCCCCTTCTTTTAGGCAGG
TTACCCACGTGTTACTCACCCGTCCGCCACTCAACTTCTTACGGTGAATGCAAGCATTCGGTGTAAGAAA
GTTTCGTTCGACTTGCATGTATTAGGCACGCCGCCAGCGTTCGTCCTGAGCCAGGNTCAAACTCTAN

### Example 2. CHARACTERIZATION: IN VITRO SAFETY ASESSMENT

### 2.1. Production of D-lactate

L- and D-lactic acid production by the strain was determined in MRS broth. One percent inoculum's from an overnight MRS culture was used and incubation proceeded for 24 h at 37° C. (until the strain reached a concentration of approx. 9 log₁₀ cfu/mL). Cells were removed by centrifugation at 12,000×g for 5 min and the concentration of L- and D-lactic acid in the supernatants was quantified by triplicate using an enzymatic kit (Roche Diagnostics, Mannheim, Germany), following the manufacturer's instructions.

The production of L-lactic acid by L. salivarius MP98 when growing in MRS broth for 16 h at 37° C. was 10.29 mg/mL (+0.53), which corresponded with a mean pH value of 3.96. As a control, the production of this lactic acid isomer by L. salivarius CECT9145 (a high acidifying strain) was 10.12 (+0.47) (pH: 4.00). D-lactic acid was not detected in the culture supernatants of L. salivarius MP98. Lack of D-lactate production is a characteristic of homofermentative lactobacilli (including L. salivarius).

### 2.2. Hemolysis

Haemolysin activity was evaluated on Columbia Blood Agar (Oxoid) containing 5% defibrinised horse blood. Single colonies were streaked onto plates and incubated at 37° C. for 24 h. Zones of clearing around colonies indicated haemolysin production.

No zones of clearing were observed around colonies of L. salivarius MP98, indicating the absence of hemolysin production by this strain.

### 2.3. Formation of Biogenic Amines

The capacity of the strain MP98 to synthesize biogenic amines (tyramine, histamine, putrescine and cadaverine) from their respective precursor amino acids (tyrosine, histidine, ornithine and lysine; Sigma-Aldrich) was evaluated using the method described by Bover-Cid and Holzapfel [2]. The strain was streaked onto the different decarboxylase medium plates and incubated for 4 days at 37° C. under aerobic and anaerobic conditions. A positive result would be indicated by a change of the medium color to purple in response to the pH shift caused by the production of the more alkaline biogenic amine from the amino acid initially included in the medium.

Parallel, the presence of genes encoding decarboxylases involved in the biosynthesis of biogenic amines was also studied. For this purpose, the tyrosine decarboxylase gene (tdcA), histidine decarboxylase gene (hdcA) and agmatine deiminase cluster (AgdDI) was checked by specific PCR using the primers pairs P2-for and P1-rev [3], JV16HC and JV17HC [4], and PTC2 and AgdDr [5], respectively. PCR conditions were those described by the respective authors. Total DNA was used as template. E. faecalis V583, which produce putrescine and tyramine, and Lentilactobacillus buchneri B301, which produce histamine, were used as positive controls.

Finally, the strains was grown for 24 h in MRS broth supplemented with 10 mM tyrosine, 13 mM of histidine or 20 mM agmatine for the detection of tyramine, histamine and putrescine production, respectively. The supernatants were filtered through a 0.2 μm pore diameter membrane, derivatyzed and analysed by thin layer chromatography (TLC) following the conditions described by García-Moruno et al [6].

No changes in the color of the media supplemented with precursor amino acids (tyrosine, histidine, ornithine and lysine) were observed around colonies of L. salivarius MP98. In adidition, the strain did not contain the tdcA, hdcA and AgdDI genes. Finally, tyramine, histamine and putrescine production was not detected using the TLC assay. Overall, all these results indicates that this strain is unable to produce biogenic amines.

### 2.4. Degradation of Mucin

The potential of the strain to degrade gastric mucine (HGM; Sigma) was evaluated in vitro as indicated by Zhou et al. [7]. Partially purified hog gastric mucine (HGM; Sigma) and agarose type I-A (Sigma) were added to a minimal anaerobic culture medium without glucose (Ruseler-van Embeden et al., 1995) at concentrations of 0.5% and 1.5% (w/V), respectively. The plates were incubated anaerobically at 37° C. for 72 h and subsequently stained with 0.1% amido black in 3.5 M acetic acid for 30 min. Then they were washed with 1.2 M acetic acid until the mucin lysis zone around the colony of positive control culture appeared. No lysis zones were observed around colonies of L. salivarius MP98, indicating that this strain was unable to degrade gastric mucin in vitro.

### 2.5. Sensitivity to Antibiotics

The sensitivity of the strains to antibiotics was determined by the E-test [AB BIODISK, Solna, Sweden) following the instructions of the manufacturer. Results were compared to the cut-off levels proposed by the European Food Safety Authority for L. salivarius [8].

The MIC values of the strain for the antibiotics to which L. salivarius strains must be evaluated according to the current EFSA guidelines are shown in Table 1. L. salivarius MP98 was susceptible to all antibiotics, with MIC values equal to, or lower than, the breakpoints defined by EFSA, with the exception of kanamycin (MIC: 128 μg/mL; EFSA cut-off value: 64 μg/mL). However, recent reports indicate that L. salivarius is intrinsically resistant to kanamycin due to lack of a transport system for this antibiotic. In addition, analysis of the whole genome of L. salivarius MP98 revealed the absence of transmissible antibiotic-resistance genes.

TABLE 1
Minimal inhibitory concentration (MIC, mg/ml) values
of 16 antibiotics^{a} to the L. salivarius MP98.

| Antibiotic^{a} | GEN | KAN | STP | NEO | TET | ERY | CLI | CHL |
|---|---|---|---|---|---|---|---|---|
| MIC value | 2 | 128 | 32 | 4 | 2 | 0.25 | 0.5 | 2 |
| Breakpoint^{b} | 16 | 64 (R^{c}) | 64 | nr | 8 | 1 | 4 | 4 |

| Antibiotic | AMP | PEN | VAN | VIR | LIN | TRM | CIP | RIF |
|---|---|---|---|---|---|---|---|---|
| MIC value | 0.5 | 0.25 | >128 | 0.25 | | 0.5 | 0.25 | 2 |
| Breakpoint^{b} | 4 | nr | nr (R^{c}) | nr | nr | nr | nr | nr |

^{a}Abbreviations: GEN, gentamycin; KAN, kanamycin; STP, streptomycin; NEO, neomycin; TET, tetracycline; ERY, erythromycin; CLI, clindamycin; CHL, chloramphenicol; AMP, ampicillin; PEN, penicillin; VAN, vancomycin; VIR, virginiamycin; LIN, linezolid; TRM, trimethoprim; CIP, ciprofloxacin; RIF, rifampicin; nr, not required by EFSA [8].
^{b}Breakpoint: microbiological breakpoints (mg/ml) that categorise Lactobacillus salivarius as resistant (microbiological breakpoints are defined as the MIC values that clearly deviate from those displayed by the normal susceptible populations .
^{c}R: the species L. salivarius is intrinsically resistant.

### Example 3. Characterization: In Vivo Safety Assessment in an Animal Model

### 3.1. Acute and Repeated Dose (4-Weeks) Oral Toxicity Studies in a Rat Model: Design of the Assay

Wistar male and female rats (Charles River Inc., Marget, Kent, UK) were acclimated for 7 days prior to study initiation with an evaluation of health status. The rats were individually housed in polycarbonate cages with sawdust bedding and maintained in environmentally controlled rooms (22±2_C and 50%±10% relative humidity) with a 12 h light-dark cycle (light from 08.00 to 20.00 h). Food (A03 rodent diet, Scientific Animal Food and Engineering, Villemoisson-sur-orge, France) and water were available ad libitum. The rats were 56-days old at the initiation of treatment.

Acute (limit test) and repeated dose (4 weeks) studies were conducted in accordance with the European Union guidelines (EC Council Regulation No. 440, 2008a,b). Both studies were undertaken in accordance with the ethics requirements and authorized by the Ethical Committee on Animal Experimentation of the Complutense University.

In the acute (limit test) study, 24 rats (12 males, 12 females) were distributed into two groups of 6 males and 6 females each. After an overnight fast each rat received skim milk (500 μl) orally (control group or Group 1), or a single oral dose of 1 @ 10¹⁰ colony-forming units (cfu) of the tested strain dissolved in 500 μl of skim milk (treated group or Group 2). Doses of the test and control products were administered by gavage. Animals were checked for clinical signs and mortality twice a day (a.m. and p.m.). At the end of a 14 days observation period, the rats were weighed, euthanized by CO₂ inhalation, exsanguinated, and necropsied.

The repeated dose (4 weeks) (limit test) study was conducted in 48 rats (24 males, 24 females) divided in four groups of 6 males and 6 females each (control group or Group 3; treated group or Group 4; satellite control group or Group 5; and satellite treated group or Group 6). Rats received a daily dose of either skim milk (Groups 3 and 5) or 1 @10⁹ cfu of the tested strain dissolved in 500 μl of skim milk (Groups 4 and 6) orally once a day over 4 weeks. Doses of the test and control articles were administered by gavage. Animals were dosed at approximately the same time each day (approximately 4-6 h into light cycle). Food but not water was withheld from 4 h before until 2 h after control and test article administration. Animals were checked for clinical signs and mortality twice a day (a.m. and p.m.). All rats of the Groups 3 and 4 were deprived of food for 18 h, weighed, euthanized by CO₂ inhalation, exsanguinated, and necropsied on Day 29. All animals of the satellite groups (Groups 5 and 6) were kept a further 14 days without treatment to detect delayed occurrence, or persistence of, or recovery from toxic effects. All rats of the Groups 5 and 6 were deprived of food for 18 h, weighed, euthanized by CO₂ inhalation, exsanguinated, and necropsied on Day 42.

### 3.2. Observations

All animals were observed twice daily for general appearance, behaviour, sings of morbidity and mortality (once before treatment and once daily thereafter). Rats were observed for their general condition and the condition of the skin and fur, eyes, nose, oral cavity, abdomen and external genitalia, evaluated for respiration rate and palpated for masses. Behavioural parameters checked were abnormal movements (tremor, convulsion, muscular contractions), reactions to handling and behaviour in open field (excitability, responsiveness to touch and to sharp noise), changes in ordinary behaviour (changes in grooming, head shaking, gyration), abnormal behaviour (autophagia, backward motion) and aggression. Body weight, body weight gain and food and water consumption were measured daily and at the end of the observation periods the rats were examined by necropsy, and the weights of the organs recorded.

### 3.3. Clinical Test Parameters

Blood samples for haematology and clinical chemistry evaluation were collected from the retro-orbital plexus from animals under light anesthesia induced by CO₂ inhalation after 14 days observation period in the acute oral study and alter 4 weeks of treatment and 14 days of recovery for the repeated dose 4 weeks safety study. EDTA was used as an anticoagulant for haematology samples and sodium citrate was used as an anticoagulant for clinical chemistry. Food was withheld for approximately 18 h before blood collection, and samples were collected early in the working day to reduce biological variation; water was provided ad libitum.

Clinical pathology parameters (haematological and clinical biochemistry) were evaluated. Most haematology variables were measured with a Coulter/CELL-DYN 3500 whole blood automated analyzer (Abbott, Chicago, IL). Blood cell smears were observed with an Olympus Microscopy BX41 (Olympus, Tokyo, Japan).

Clinical chemistry parameters were evaluated with a spectrophotometer Konelab PRIME 30 (Thermofisher Scientific Inc. Waltham, MA, USA) and special biochemistry parameters with a clinical chemistry analyzer AU640 (Olympus, Tokyo, Japan). Coagulation parameters were analyzed with a coagulation analyzer Coatron M1 (Teco Medical Instruments, GMBH, Neufahrn, Germany).

### 3.4. Anatomical Pathology

All rats were euthanized by CO₂ inhalation and necropsied. The necropsy included a macroscopic examination of the external surface of the body, all orifices, the cranial cavity, the brain and spinal cord, the nasal cavity and paranasal sinuses, and the thoracic, abdominal, and pelvic cavities and viscera. Descriptions of all macroscopic abnormalities were recorded. Samples of the following tissues and organs were collected from all animals at necropsy and fixed in neutral phosphate-buffered 4% formaldehyde solution: adrenal glands, brain, heart, ileum, jejunum, caecum, colon, duodenum, rectum, stomach, oesophagus, trachea, kidneys, liver, lungs, pancreas, spleen, skin, testicles with epididymes, ovaries with oviducts, bone marrow, thymus, thyroid and parathyroid glands, seminal vesicles, urinary bladder and uterus. The organ: body weight ratios were calculated. All organ and tissue simples for histopathological examination were processed, embedded in paraffin, cut at an approximate thickness of 2 to 4 μm, and stained with hematoxylin and eosin. Slides of all organs and tissues listed above were collected from all animals of the control and treated groups.

### 3.5. Bacterial Translocation

Bacterial translocation was analysed in blood, liver and spleen. Blood (50 μl) were cultured in de Man, Rogosa, Sharpe (MRS) agar medium and incubated at 37° C. during 48 h anaerobically. Tissue samples were homogenized in buffered peptone water (1 g/ml) and 100 μl of the resulting homogenates were cultured on MRS agar as previously mentioned. After 48 h, the plates were checked for the presence of lactobacilli. Positive growth on MRS agar plates was defined by the presence of even a single colony.

### 3.6. Total Liver Glutathione (GSH) Concentration

A portion of 100 mg of liver from each mouse were homogenized in a 7.5% trichloroacetic acid solution and homogenates were centrifuged at 3,000 μg for 10 min at 4° C. Total glutathione concentration was measured in the supernatants using a colorimetric commercial kit (OxisResearch, Portland, OR). Briefly, 40 μl of the homogenates or the standards were added to each well of a microtiter plate, together with 40 ml of a reducing agent (tris (2-carboxyethyl) phosphine in HCl), 40 ml of a chromogen (1-methyl-3-chloro-7-trifluoromethylquinolinium methylsulfate in HCl) and 40 ml of color developer (NaOH). After an incubation at room temperature and in the dark for 30 min, optical density was measured at 415 nm using a microplate spectrophotometer (Bio-Rad Laboratories, Hercules CA).

### 3.7. Statistical Analysis

All data are expressed as means±standard error of the mean (SEM) of 6 determinations (i.e. 6 males and 6 females). Differences between control and treated groups were evaluated with a one-way analysis of variance (ANOVA) followed by Dunnett's test (1995), and differences were considered significant at P<0.05.

### 3.8. Results: Acute Oral Toxicity in Rats

No abnormal clinical signs, behavioural changes, body weight changes, macroscopic findings, or organ weight changes were observed. All animals survived the 2-week observation period. There were no statistical differences in body weights among groups. Similarly, no statistically significant differences in body weight gain, food and water consumption were noted. Body weight, daily body weight gain, food and water consumption thus were unaffected by the treatment (single oral dose of 1@10¹⁰ cfu of the tested strain).

The haematological and clinical chemistry parameters assessed 2 weeks after administration of the strain as a single oral dose of 1@10¹⁰ cfu were not significantly different compared with those of controls (Tables 2 and 3). No treatment-related changes were noted.

TABLE 2
Haematological parameters in rats after the 2-week observation period
following a single oral dose of L. salivarius MP98 at 10 to 10 cfu

| | Group 1 (control) | | Group 2 (probiotic) | |
|---|---|---|---|---|
| Parameters* | Female | Male | Female | Male |
| RBC (×10⁶/ml) | 8.31 ± 0.29 | 8.35 ± 0.25 | 8.34 ± 0.35 | 8.30 ± 0.41 |
| Haemoglobin (g/dl) | 14.79 ± 0.59 | 14.88 ± 0.44 | 14.83 ± 0.53 | 14.86 ± 0.62 |
| Haematocrit (%) | 47.37 ± 0.62 | 47.62 ± 0.56 | 47.47 ± 0.48 | 47.67 ± 0.61 |
| MCV (fl) | 57.03 ± 0.66 | 58.45 ± 0.82 | 56.93 ± 0.88 | 58.40 ± 1.27 |
| MCH (pg) | 17.75 ± 0.47 | 17.69 ± 0.61 | 17.70 ± 0.48 | 17.73 ± 0.66 |
| MCHC (g/dl) | 31.53 ± 0.44 | 31.85 ± 0.46 | 31.55 ± 0.46 | 31.80 ± 0.23 |
| RDW (%) | 15.73 ± 0.42 | 15.79 ± 0.34 | 15.45 ± 0.36 | 15.75 ± 0.40 |
| WBC (×10³/ml) | 9.54 ± 0.77 | 9.91 ± 0.86 | 9.48 ± 0.64 | 9.83 ± 0.80 |
| Banded neutrophils | 0.00 ± 0.00 | 0.00 ± 0.00 | 0.00 ± 0.00 | 0.00 ± 0.00 |
| (×10³/ml) | | | | |
| Neutrophils (×10³/ml) | 1.51 ± 0.21 | 1.47 ± 0.23 | 1.46 ± 0.32 | 1.50 ± 0.24 |
| Eosinophils (×10³/ml) | 0.05 ± 0.02 | 0.06 ± 0.01 | 0.04 ± 0.01 | 0.05 ± 0.02 |
| Lymphocytes (×10³/ml) | 6.99 ± 0.57 | 9.09 ± 0.62 | 7.01 ± 0.66 | 9.00 ± 0.70 |
| Monocytes (×10³/ml) | 0.30 ± 0.08 | 0.34 ± 0.09 | 0.29 ± 0.07 | 0.35 ± 0.08 |
| Basophils (×10³/ml) | 0.00 ± 0.00 | 0.00 ± 0.00 | 0.00 ± 0.00 | 0.00 ± 0.00 |
| Platelets (×10³/ml) | 709.01 ± 34.12 | 727.83 ± 28.92 | 714.67 ± 39.21 | 732.16 ± 37.08 |
| MPV (fl) | 6.36 ± 0.09 | 6.84 ± 0.12 | 6.29 ± 0.12 | 6.79 ± 0.13 |

Differences between the treated group and the control group were not significant.
*Data are expressed as mean ± SEM (n = 6 animals/sex group).

TABLE 3
Clinical chemistry parameters in rats after the 2-week observation period
following a single oral dose of L. salivarius MP98 at 10¹⁰ cfu

| | Group 1 (control) | | Group 2 (probiotic) | |
|---|---|---|---|---|
| Parameters* | Female | Male | Female | Male |
| Glucose (mg/dl) | 120.00 ± 4.23 | 122.18 ± 4.32 | 118.96 ± 4.26 | 121.17 ± 5.08 |
| Urea nitrogen (mg/dl) | 49.17 ± 4.16 | 44.01 ± 3.88 | 48.10 ± 2.51 | 43.37 ± 3.67 |
| Creatinine (mg/dl) | 0.60 ± 0.08 | 0.54 ± 0.08 | 0.58 ± 0.07 | 0.52 ± 0.07 |
| Total protein (g/dl) | 7.51 ± 0.22 | 6.78 ± 0.24 | 7.54 ± 0.32 | 6.82 ± 0.30 |
| Total bilirubin (mg/dl) | 0.17 ± 0.04 | 0.18 ± 0.04 | 0.16 ± 0.05 | 0.18 ± 0.04 |
| Calcium (mg/dl) | 11.29 ± 0.52 | 11.88 ± 0.42 | 11.98 ± 0.64 | 11.83 ± 0.56 |
| Sodium (mEq/l) | 146.23 ± 6.12 | 149.04 ± 7.24 | 145.01 ± 7.50 | 148.14 ± 7.87 |
| Potassium (mEq/l) | 5.87 ± 0.46 | 6.43 ± 0.63 | 5.84 ± 0.53 | 6.47 ± 0.59 |
| ASAT (u/l) 1 | 150.08 ± 7.88 | 159.34 ± 8.26 | 151.00 ± 8.04 | 157.31 ± 7.96 |
| ALAT (u/l) | 62.77 ± 5.80 | 70.09 ± 6.00 | 61.94 ± 5.67 | 70.74 ± 6.03 |
| Alkaline phosphatase | 277.32 ± 35.23 | 823.83 ± 39.67 | 270.86 ± 37.44 | 820.63 ± 40.12 |
| (u/l) | | | | |
| Triglyceride (mg/dl) | 107.12 ± 8.48 | 128.01 ± 8.33 | 104.13 ± 9.33 | 126.65 ± 9.27 |
| Cholesterol (mg/dl) | 67.21 ± 4.47 | 63.21 ± 5.79 | 65.05 ± 6.04 | 62.06 ± 5.98 |
| HDL (mg/dl) | 47.32 ± 3.76 | 42.09 ± 4.06 | 45.86 ± 5.02 | 42.11 ± 5.52 |
| LDL (mg/dl) | 7.77 ± 0.86 | 8.44 ± 0.97 | 7.64 ± 0.77 | 8.45 ± 0.83 |

Differences between the treated group and the control group were not significant.
*Data are expressed as mean ± SEM (n = 6 animals/sex group).

There were no statistical differences in organ weight or tissue: body weight ratios related to any of the tested strains (data not shown). Any tested strain preparation was associated with any incidence of macroscopic and microscopic changes. No treatment-related histopathological changes were observed 2 weeks after administration of any of the strains as a single oral dose of 1×10¹⁰ cfu. Therefore, the tested strains has a low order of acute toxicity and the oral lethal dose (LD50) for male and female rats is higher than 1×10¹⁰ cfu.

### 3.9. Results: Repeated Dose (4 Weeks) Oral Toxicity in Rats

No mortality was observed. No treatment-related changes in the general condition and external appearance were observed in male and female rats daily treated with 1×10⁹ cfu of any of the tested strains. The development of the animals during the experimental period corresponded to their species and age. There was no significant difference in body weight or body weight gain among groups treated with any of the tested strains in comparison to the control groups at any time point of the experimental period. All treated groups consumed similar amounts of food and water (data not shown) to that of the corresponding control groups.

All haematology data were within normal limits and differences between groups were no observed (Table 4). Clinical chemistry data showed no treatment-related alterations at the end of 4-weeks treatment period (Table 5). Individual values and group mean values were within the physiologic ranges. After 14 days without treatment to detect delayed occurrence of potential toxic effects, there were no treatment-related changes in haematological and clinical test parameters (Table 4 and 5; satellite control group or Group 5 and satellite treated group or Group 6).

TABLE 4
Haematological parameters in rats after repeated (4 weeks) oral doses of L. salivarius MP98 at 10⁹ cfu

| | | | | | Group 5 | | Group 6 | |
|---|---|---|---|---|---|---|---|---|
| | Group 3 | | Group 4 | | (satellite | | (satellite | |
| | (control) | | (probiotic) | | control) | | probiotic) | |
| Parameters* | Female | Male | Female | Male | Female | Male | Female | Male |
| RBC(×10⁶/ml) | 8.72 ± | 8.81 ± | 8.69 ± | 8.83 ± | 8.71 ± | 8.82 ± | 8.68 ± | 8.85 ± |
| | 0.14 | 0.13 | 0.15 | 0.12 | 0.14 | 0.15 | 0.11 | 0.15 |
| Haemoglobin(g/dl) | 16.60 ± | 16.74 ± | 15.62 ± | 16.81 ± | 16.57 ± | 16.80 ± | 16.61 ± | 16.82 ± |
| | 0.41 | 0.61 | 0.52 | 0.47 | 0.54 | 0.59 | 0.61 | 0.58 |
| Haematocrit(%) | 45.99 ± | 47.08 ± | 45.88 ± | 47.03 ± | 46.04 ± | 46.99 ± | 46.00 ± | 47.06 ± |
| | 2.27 | 3.22 | 2.67 | 2.57 | 3.29 | 3.83 | 2.92 | 2.99 |
| MCV (fl) | 51.72 ± | 52.94 ± | 51.68 ± | 52.89 ± | 51.62 ± | 53.00 ± | 52.71 ± | 52.83 ± |
| | 0.89 | 0.82 | 0.79 | 0.96 | 0.88 | 0.96 | 1.34 | 0.94 |
| MCH (pg) | 18.28 ± | 18.77 ± | 18.24 ± | 18.75 ± | 18.32 ± | 18.76 ± | 18.26 ± | 18.74 ± |
| | 0.51 | 0.49 | 0.51 | 0.61 | 0.57 | 0.69 | 0.53 | 0.64 |
| MCHC (g/dl) | 34.87 ± | 35.41 ± | 34.97 ± | 35.44 ± | 35.02 ± | 35.17 ± | 34.99 ± | 35.42 ± |
| | 0.92 | 0.81 | 0.76 | 0.85 | 0.98 | 0.86 | 0.79 | 1.01 |
| RDW (%) | 18.00 ± | 17.94 ± | 17.91 ± | 18.02 ± | 18.04 ± | 17.93 ± | 18.02 ± | 18.03 ± |
| | 0.43 | 0.52 | 0.66 | 0.47 | 0.53 | 0.49 | 0.62 | 0.74 |
| WBC (10³/II) | 7.65 ± | 9.97 ± | 7.62 ± | 10.11 ± | 7.68 ± | 9.99 ± | 7.62 ± | 9.95 ± |
| | 0.67 | 0.68 | 0.78 | 0.55 | 0.58 | 0.75 | 0.47 | 0.74 |
| Banded | 0.00 ± | 0.00 ± | 0.00 ± | 0.00 ± | 0.00 ± | 0.00 ± | 0.00 ± | 0.00 ± |
| neutrophils | 0.00 | 0.00 | 0.00 | 0.00 | 0.00 | 0.00 | 0.00 | 0.00 |
| (×10³/ml) | | | | | | | | |
| Neutrophils | 1.04 ± | 1.47 ± | 1.05 ± | 1.49 ± | 1.03 ± | 1.45 ± | 1.00 ± | 1.46 ± |
| (×10³/ml) | 0.14 | 0.13 | 0.12 | 0.11 | 0.10 | 10.13 | 0.14 | 0.12 |
| Eosinophils | 0.12 ± | 0.17 ± | 0.10 ± | 0.15 ± | 0.11 ± | 0.15 ± | 0.09 ± | 0.14 ± |
| (×10³/ml) | 0.02 | 0.03 | 0.032 | 0.03 | 0.01 | 10.02 | 0.02 | 0.03 |
| Lymphocytes | 7.77 ± | 7.95 ± | 7.84 ± | 8.02 ± | 7.93 ± | 7.99 ± | 7.69 ± | 8.00 ± |
| (×10³/ml) | 0.59 | 0.56 | 0.62 | 0.64 | 0.62 | 0.67 | 0.55 | 0.61 |
| Monocytes | 0.23 ± | 0.40 ± | 0.25 ± | 0.44 ± | 0.26 ± | 0.43 ± | 0.24 ± | 0.42 ± |
| (×10³/ml) | 0.04 | 0.05 | 0.04 | 0.04 | 0.02 | 0.05 | 0.03 | 0.06 |
| Basophils | 0.00 ± | 0.00 ± | 0.00 ± | 0.00 ± | 0.00 ± | 0.00 ± | 0.00 ± | 0.00 ± |
| (×10³/ml) | 0.00 | 0.00 | 0.00 | 0.00 | 0.00 | 0.00 | 0.00 | 0.00 |
| Platelets | 765.17 ± | 793.67 ± | 760.35 ± | 791.39 ± | 760.72 ± | 796.77 ± | 767.06 ± | 798.04 ± |
| (×10³/ml) | 40.19 | 38.97 | 41.13 | 56.01 | 38.83 | 35.50 | 37.82 | 34.06 |
| MPV (fl) | 8.64 ± | 8.75 ± | 8.67 ± | 8.73 ± | 8.65 ± | 8.71 ± | 8.68 ± | 8.74 ± |
| | 0.23 | 0.25 | 0.30 | 0.29 | 0.27 | 0.34 | 0.33 | 0.28 |
| Prothrombin | 17.62 ± | 17.9 ± | 17.73 ± | 18.03 ± | 17.72 ± | 18.07 ± | 17.55 ± | 18.03 ± |
| time (s) | 0.34 | 0.39 | 0.27 | 0.34 | 0.35 | 0.31 | 0.37 | 0.35 |
| Thromboplastin | 30.09 ± | 25.00 ± | 31.01 ± | 24.94 ± | 29.58 ± | 25.32 ± | 29.97 ± | 24.97 ± |
| partialtime (s) | 1.72 | 1.83 | 1.90 | 1.77 | 1.93 | 1.79 | 1.86 | 1.92 |
| Fibrinogen | 235.30 ± | 305.44 ± | 229.07 ± | 297.29 ± | 243.38 ± | 300.43 ± | 241.06 ± | 311.03 ± |
| (mg/dl) | 11.37 | 12.52 | 13.55 | 12.36 | 15.04 | 24.69 | 15.71 | 19.72 |

Group 3 and Group 4 were sacrificed after treatment on Day 29. Differences between treated group (Group 4) and the control group (Group 3) were not significant.
Group 5 and Group 6 were sacrificed on Day 42 after an additional observation period of 14 days without treatment. Differences between satellite treated group (Group 6) and the satellite control group (Group 5) were not significant.
*Data are expressed as mean ± SEM (n = 6 animals/sex group).

TABLE 5
Clinical chemistry in rats after repeated (4 weeks) oral doses of L. salivarius MP98 at 10⁹ cfu

| | | | | | Group 5 | | Group 4 | |
|---|---|---|---|---|---|---|---|---|
| | Group 3 | | Group 4 | | (satellite | | (satellite | |
| | (control) | | (probiotic) | | control) | | probiotic) | |
| Parameters* | Female | Male | Female | Male | Female | Male | Female | Male |
| Glucose | 105.13 ± | 121.33 ± | 103.66 ± | 120.73 ± | 106.74 ± | 119.35 ± | 105.97 ± | 121.02 ± |
| (mg/dl) | 4.14 | 4.06 | 3.78 | 3.97 | 4.59 | 5.01 | 4.49 | 5.06 |
| Urea | 42.17 ± | 39.37 ± | 41.74 ± | 38.80 ± | 41.28 ± | 41.67 ± | 42.05 ± | 39.73 ± |
| nitrogen | 1.92 | 1.72 | 2.12 | 2.43 | 2.61 | 2.93 | 2.88 | 2.95 |
| (mg/dl) | | | | | | | | |
| Creatinine | 0.68 ± | 0.65 ± | 0.64 ± | 0.63 ± | 0.68 ± | 0.64 ± | 0.67 ± | 0.64 ± |
| (mg/dl) | 0.06 | 0.05 | 0.04 | 0.04 | 0.06 | 0.05 | 0.07 | 0.05 |
| Albumine | 3.80 ± | 3.60 ± | 3.77 ± | 3.57 ± | 3.83 ± | 3.52 ± | 3.79 ± | 3.55 ± |
| (g/dl) | 0.26 | 0.24 | 0.27 | 0.35 | 0.28 | 0.33 | 0.26 | 0.25 |
| Total | 6.74 ± | 6.47 ± | 6.67 ± | 6.50 ± | 6.77 ± | 6.54 ± | 6.73 ± | 6.55 ± |
| protein | 0.25 | 0.29 | 0.31 | 0.24 | 0.27 | 0.26 | 0.31 | 0.19 |
| (g/dl) | | | | | | | | |
| Total bilirubin | 0.19 ± | 0.18 ± | 0.19 ± | 0.17 ± | 0.20 ± | 0.19 ± | 0.20 ± | 0.21 ± |
| (mg/dl) | 0.03 | 0.04 | 0.03 | 0.03 | 0.04 | 0.03 | 0.03 | 0.04 |
| Calcium | 12.27 ± | 11.82 ± | 12.44 ± | 12.07 ± | 12.37 ± | 12.02 ± | 12.32 ± | 12.00 ± |
| (mg/dl) | 0.35 | 0.37 | 0.36 | 0.37 | 0.39 | 0.37 | 0.36 | 0.41 |
| Sodium | 138.70 ± | 139.07 ± | 137.85 ± | 138.97 ± | 139.02 ± | 140.03 ± | 140.34 ± | 140.92 ± |
| (mEq/l) | 3.29 | 3.02 | 3.48 | 3.49 | 3.67 | 3.66 | 3.84 | 3.92 |
| Potassium | 6.00 ± | 6.12 ± | 5.99 ± | 6.02 ± | 6.10 ± | 5.97 ± | 6.03 ± | 5.98 ± |
| (mEq/l) | 0.35 | 0.31 | 0.28 | 0.29 | 0.30 | 0.28 | 0.29 | 0.30 |
| ASAT (u/l) | 148.22 ± | 141.23 ± | 145.05 ± | 140.08 ± | 144.90 ± | 142.08 ± | 144.98 ± | 141.92 ± |
| | 6.61 | 6.80 | 7.04 | 6.43 | 6.32 | 6.83 | 7.00 | 6.84 |
| ALAT (u/l) | 45.97 ± | 43.27 ± | 45.77 ± | 42.96 ± | 45.63 ± | 43.53 ± | 45.50 ± | 43.67 ± |
| | 2.05 | 2.32 | 2.14 | 2.53 | 2.68 | 3.00 | 2.83 | 2.76 |
| Alkaline | 299.67 ± | 477.13 ± | 287.76 ± | 469.05 ± | 294.99 ± | 472.11 ± | 299.71 ± | 474.00 ± |
| phosphatase(u/l) | 20.21 | 29.80 | 27.54 | 126.90 | 24.73 | 26.00 | 37.56 | 30.48 |
| Triglyceride | 140.03 ± | 177.87 ± | 139.01 ± | 173.01 ± | 141.58 ± | 175.00 ± | 141.99 ± | 174.38 ± |
| (mg/dl) | 11.35 | 11.29 | 12.23 | 13.11 | 12.93 | 15.78 | 15.77 | 14.83 |
| Cholesterol | 58.54 ± | 65.76 ± | 57.01 ± | 64.05 ± | 58.34 ± | 65.48 ± | 57.83 ± | 64.97 ± |
| (mg/dl) | 2.51 | 2.54 | 2.95 | 2.67 | 3.51 | 3.71 | 3.87 | 3.69 |
| HDL (mg/dl) | 39.52 ± | 37.23 ± | 38.87 ± | 37.08 ± | 39.06 ± | 38.23 ± | 38.75 ± | 37.55 ± |
| | 1.66 | 1.54 | 1.92 | 1.98 | 1.88 | 3.00 | 2.72 | 2.85 |
| LDL (mg/dl) | 7.63 ± | 8.04 ± | 7.56 ± | 8.17 ± | 7.53 ± | 8.55 ± | 8.27 ± | 8.23 ± |
| | 0.98 | 0.85 | 0.87 | 1.03 | 1.15 | 1.05 | 0.96 | 1.147 |
| Lipoprotein A | <2 | <2 | <2 | <2 | <2 | <2 | <2 | <2 |
| (mg/dl) | | | | | | | | |

Group 3 and Group 4 were sacrificed after treatment on Day 29. Differences between treated group (Group 4) and the control group (Group 3) were not significant.
Group 5 and Group 6 were sacrificed on Day 42 after an additional observation period of 14 days without treatment. Differences between satellite treated group (Group 6) and the satellite control group (Group 5) were not significant.
*Data are expressed as mean ± SEM (n = 6 animals/sex group).

The necropsy performed on day 29 after the last dose of the strain (Group 4) and on day 42 after 14 days without any treatment (Group 6) did not reveal any gross pathological changes or any differences in organ weights in comparison to the corresponding control groups. Mean organ weights and rate body weight: organ are presented in Table 6. After 4-weeks of treatment, there were no histopathological findings in the organs examined considered being treatment related in male and female rats (data not shown). There were also no treatment-related histopathological findings in the satellite treated group (Group 6) (data not shown).

TABLE 6
Mean organ weights and rate body weight/organ in rats after repeated (4 weeks) oral doses of L. salivarius MP98 at 10 to 9 cfu.

| | Group 3 (control) | | Group 4 (probiotic) | | Group 5 (satellite control) | | Group 4 (satellite probiotic) | |
|---|---|---|---|---|---|---|---|---|
| Parameters* | Female | Male | Female | Male | Female | Male | Female | Male |
| Body weight (g) | 238.81 ± 7.16 | 327.21 ± 9.35 | 237.00 ± 8.12 | 333.04 ± 8.02 | 243.66 ± 7.56 | 383.75 ± 7.97 | 245.04 ± 6.48 | 378.64 ± 8.31 |
| Increase body weight (g) | 46.90 ± 3.81 | 117.46 ± 7.25 | 46.06 ± 5.78 | 120.70 ± 5.33 | 49.37 ± 4.77 | 173.30 ± 5.57 | 50.03 ± 3.77 | 169.28 ± 8.92 |
| Brain weight (g) | 1.80 ± 0.05 | 1.89 ± 0.06 | 1.82 ± 0.07 | 1.85 ± 0.06 | 1.77 ± 0.07 | 1.91 ± 0.06 | 1.77 ± 0.05 | 1.87 ± 0.07 |
| Rate body weight/brain (%) | 0.75 ± 0.04 | 0.57 ± 0.04 | 0.76 ± 0.05 | 0.55 ± 0.04 | 0.72 ± 0.06 | 0.49 ± 0.04 | 0.72 ± 0.05 | 0.49 ± 0.04 |
| Thymus weight (g) | 0.42 ± 0.03 | 0.68 ± 0.06 | 0.44 ± 0.04 | 0.64 ± 0.07 | 0.43 ± 0.04 | 0.55 ± 0.04 | 0.43 ± 0.05 | 0.58 ± 0.06 |
| Rate body weight/thymus (%) | 0.16 ± 0.03 | 0.20 ± 0.04 | 0.17 ± 0.04 | 0.18 ± 0.04 | 0.17 ± 0.05 | 0.15 ± 0.06 | 0.17 ± 0.04 | 0.15 ± 0.06 |
| Heart weight (g) | 0.64 ± 0.06 | 0.94 ± 0.08 | 0.66 ± 0.07 | 0.93 ± 0.09 | 0.69 ± 0.08 | 0.93 ± 0.07 | 0.70 ± 0.06 | 0.95 ± 0.07 |
| Rate body weight/heart (%) | 0.26 ± 0.03 | 0.28 ± 0.03 | 0.27 ± 0.04 | 0.27 ± 0.05 | 0.28 ± 0.05 | 0.25 ± 0.03 | 0.28 ± 0.04 | 0.24 ± 0.03 |
| Right lung weight (g) | 0.55 ± 0.04 | 0.76 ± 0.06 | 0.58 ± 0.05 | 0.75 ± 0.07 | 0.57 ± 0.06 | 0.84 ± 0.06 | 0.59 ± 0.04 | 0.81 ± 0.07 |
| Rate body weight/right lung (%) | 0.22 ± 0.03 | 0.23 ± 0.02 | 0.24 ± 0.02 | 0.23 ± 0.03 | 0.23 ± 0.02 | 0.22 ± 0.04 | 0.24 ± 0.01 | 0.22 ± 0.02 |
| Left lung weight (g) | 0.34 ± 0.03 | 0.45 ± 0.04 | 0.33 ± 0.03 | 0.43 ± 0.03 | 0.34 ± 0.02 | 0.47 ± 0.04 | 0.34 ± 0.03 | 0.48 ± 0.03 |
| Rate body weight/left lung (%) | 0.15 ± 0.02 | 0.14 ± 0.02 | 0.14± 0.01 | 0.15 ± 0.02 | 0.14 ± 0.01 | 0.13 ± 0.02 | 0.14 ± 0.02 | 0.13 ± 0.02 |
| Liver weight (g) | 8.16 ± 0.40 | 12.34 ± 0.32 | 8.21 ± 0.43 | 12.35 ± 0.35 | 7.10 ± 0.31 | 13.58 ± 0.28 | 6.98 ± 0.29 | 13.44 ± 0.34 |
| Rate body weight/liver (%) | 3.41 ± 0.27 | 3.77 ± 0.29 | 3.46 ± 0.22 | 3.70 ± 0.28 | 2.92 ± 0.18 | 3.55 ± 0.22 | 2.85 ± 0.21 | 3.55 ± 0.23 |
| Spleen weight (g) | 0.52 ± 0.05 | 0.74 ± 0.04 | 0.55 ± 0.05 | 0.75 ± 0.04 | 0.52 ± 0.04 | 0.78 ± 0.05 | 0.50 ± 0.03 | 0.75 ± 0.05 |
| Rate body weight/spleen (%) | 0.21 ± 0.03 | 0.23 ± 0.03 | 0.24 ± 0.02 | 0.23 ± 0.02 | 0.22 ± 0.03 | 0.21 ± 0.04 | 0.21 ± 0.03 | 0.20 ± 0.02 |
| Pancreas weight (g) | 0.26 ± 0.04 | 0.45 ± 0.05 | 0.23 ± 0.04 | 0.47 ± 0.06 | 0.29 ± 0.03 | 0.42 ± 0.04 | 0.26 ± 0.03 | 0.41 ± 0.04 |
| Rate body weight/pancreas (%) | 0.10 ± 0.02 | 0.14 ± 0.01 | 0.10 ± 0.02 | 0.14 ± 0.02 | 0.12 ± 0.01 | 0.11 ± 0.01 | 0.11 ± 0.01 | 0.11 ± 0.01 |
| Right kidney weight (g) | 0.71 ± 0.07 | 1.11 ± 0.07 | 0.69 ± 0.05 | 1.10 ± 0.08 | 0.70 ± 0.04 | 1.09 ± 0.07 | 0.67 ± 0.03 | 1.08 ± 0.08 |
| Rate body weight/right kidney (%) | 0.29 ± 0.03 | 0.34 ± 0.02 | 0.30 ± 0.01 | 0.33 ± 0.02 | 0.29 ± 0.01 | 0.29 ± 0.01 | 0.28 ± 0.01 | 0.29 ± 0.01 |
| Left kidney weight (g) | 0.73 ± 0.06 | 1.10 ± 0.08 | 0.72 ± 0.04 | 1.08 ± 0.07 | 0.71 ± 0.03 | 1.09 ± 0.06 | 0.70 ± 0.04 | 1.08 ± 0.07 |
| Rate body weight/left kidney (%) | 0.32 ± 0.03 | 0.34 ± 0.05 | 0.31 ± 0.02 | 0.33 ± 0.03 | 0.29 ± 0.03 | 0.29 ± 0.02 | 0.29 ± 0.03 | 0.29 ± 0.02 |
| Right adrenal gland weight (g) | 0.04 ± 0.010 | 0.04 ± 0.011 | 0.05 ± 0.011 | 0.04 ± 0.012 | 0.04 ± 0.011 | 0.04 ± 0.013 | 0.04 ± 0.014 | 0.05 ± 0.014 |
| Rate body weight/right adrenal gland (% | 0.02 ± 0.012 | 0.01 ± 0.012 | 0.02 ± 0.012 | 0.01 ± 0.012 | 0.02 ± 0.012 | 0.01 ± 0.012 | 0.02 ± 0.013 | 0.01 ± 0.014 |
| Left adrenal gland weight (g) | 0.04 ± 0.012 | 0.04 ± 0.012 | 0.05 ± 0.013 | 0.04 ± 0.013 | 0.04 ± 0.012 | 0.04 ± 0.013 | 0.04 ± 0.012 | 0.05 ± 0.011 |
| Rate body weight/left adrenal gland (%) | 0.02 ± 0.012 | 0.01 ± 0.014 | 0.02 ± 0.012 | 0.01 ± 0.011 | 0.02 ± 0.013 | 0.01 ± 0.011 | 0.02 ± 0.012 | 10.01 ± 0.011 |
| Right testicle weight (g) | | 1.72 ± 0.15 | | 1.69 ± 0.13 | | 1.74 ± 0.14 | | 1.74 ± 0.14 |
| Rate body weight/right testicle (%) | | 0.52 ± 0.03 | | 0.51 ± 0.05 | | 0.45 ± 0.04 | | 0.46 ± 0.03 |
| Left testicle weight (g) | | 1.74 ± 0.07 | | 1.68 ± 0.08 | | 1.77 ± 0.09 | | 1.73 ± 0.06 |
| Rate body weight/left testicle (%) | | 0.53 ± 0.04 | | 0.50 ± 0.04 | | 0.46 ± 0.03 | | 0.46 ± 0.03 |
| Bone marrow weight (g) | 0.07 ± 0.004 | 0.08 ± 0.010 | 0.09 ± 0.006 | 0.08 ± 0.011 | 0.08 ± 0.012 | 0.08 ± 0.011 | 0.09 ± 0.012 | 0.09 ± 0.010 |
| Rate body weight/bone marrow (%) | 0.03 ± 0.003 | 0.02 ± 0.002 | 0.04 ± 0.004 | 0.02 ± 0.002 | 0.03 ± 0.003 | 0.02 ± 0.004 | 0.04 ± 0.005 | 10.02 ± 0.003 |

Group 3 and Group 4 were sacrificed after treatment on Day 29. Differences between treated group (Group 4) and the control group (Group 3) were not significant.
Group 5 and Group 6 were sacrificed on Day 42 after an additional observation period of 14 days without treatment. Differences between satellite treated group (Group 6) and the satellite control group (Group 5) were not significant.
*Data are expressed as mean ± SEM (n = 6 animals/sex group).

The no-observed-adverse-effect level in this repeated dose (4 weeks) oral toxicity study was the dose tested, i.e. 1×10⁹ cfu for the tested strain.

### 3.10. Results: Total Liver Glutathione (GSH) Concentration

In order to determine changes in the antioxidant defence because of the treatment, liver GSH concentration was determined. No significant differences in liver GSH concentration were observed between control and treated groups (9.42±1.32 versus 9.34±1.41 mmol/g, P>0.1). This indicates that treatment with the tested strain did not cause oxidative stress to rats and is consistent with the absence of bacteraemia since no lactobacilli could be isolated from blood, liver or spleen of the rats. It suggests that the tested strain do not cause either local or systemic infections in rats.

### 3.11. Results: Presence of L. salivarius MP98 in the Rats' Feces

L. salivarius MP98 could be isolated from fecal material and vaginal swabs samples of the treated animals (probiotic groups) at the end of the treatment. The concentration oscillated between 4.84 and 7.94 log₁₀ cfu/g of colonic material, and between 3.53 and 6.29 log₁₀ cfu/swab in the vaginal samples. The strain could not be detected in any sample from the control group.

### 3.12. Conclusion

The present study has evaluated the potencial acute and repeated dose (4 weeks) oral toxicity of L. salivarius MP98. Based on the present results, the maximal oral dose of the tested strain in the 4-weeks repeated dose (1×10⁹ cfu) study did not show any difference among controls and treated animals, thus representing a dose level of non-observable toxic effects (NOAEL). Overall, no strain-related toxicity has been observed after single oral limit dose (1×10¹⁰ cfu) suggesting a low potential oral toxicity.

### Example 4. Characterization: General Functional Properties

### 4.1. Survival after In Vitro Exposure to Saliva and Gastrointestinal-Like Conditions

The survival of the strain to conditions resembling those found in the human digestive tract (saliva, human stomach and small intestine) was assessed in the in vitro system described by Marteau et al. [9], with the modifications reported by Martin et al. [10]. For this purpose, the strain was vehiculated in UHT-treated milk (25 mL) at a concentration of 10⁹ CFU/mL. Subsequently, it was diluted in 5 ml of a sterile electrolyte solution containing 6.2 g/L of NaCl, 2.2 g/L of KCl, 0.22 g/L of CaCl₂), and 1.2 g/L of NaHCO₃ to simulate the in vivo dilution by saliva. Then, 5 mL of porcine gastric juice was added and the mixture was incubated at 37° C. with agitation. The pH curve in the stomach-resembling compartment was controlled to reproduce the values found in humans after yogurt consumption [11]: pH 5.0 at initiation, pH 4.1 at 20 min, pH 3.0 at 40 min, pH 2.1 at 60 min, and pH 1.8 after 80 min. Fractions were successively taken from this compartment at 0, 20, 40, 60, and 80 min, in a manner that simulate the normal gastric emptying. After adjusting their pH to 6.5±0.2 with 1 M NaHCO₃, they were mixed with 10 mL of a sterile electrolyte solution containing 5 g/L of NaCl, 0.6 g/L of KCl, 0.3 g/L of CaCl₂), 4% of porcine bile, and 7% of pancreatin (Sigma, St. Louis, Mo), which simulates the content of the duodenal juice. After 120 min of exposition, bacterial survival was determined by plating the samples onto MRS agar plates, which were incubated at 37° C. for 48 h. The assay was performed by triplicate. Lacticaseibacillus rhamnosus GG was used as a control strain because of its high rate of survival during transit through the human gastrointestinal tract.

The results showed that exposure to a saliva-like solution had no deleterious effect on the strain while the survival rate after a transit through the stomach- and small intestine-like compartments was at approximately 63% (+2.6) of the population initially inoculated, a value similar to that achieved by L. rhamnosus GG (61.7%±2.3). As a consequence, the MP98 strain showed a high survival rate to the conditions found in the human gastrointestinal tract.

### 4.2. Adhesion to Caco-2 and HT-29 Cells

The ability of the strain to adhere to HT-29 and Caco-2 cells was evaluated as described by Coconnier et al. with the modifications reported by Martin et al. [10]. HT-29 and Caco-2 were cultured to confluence in 2 mL of DMEM medium (PAA, Linz, Austria) containing 25 mM of glucose, 1 mM of sodium pyruvate and supplemented with 10% heat-inactivated foetal calf serum, 2 mM of L-glutamine and 1% of a non-essential amino acid preparation. At day 10 after the confluence, 1 mL of the medium was replaced with 1 mL of DMEM containing 10⁸ CFU/mL of the MP98 strain. The inoculated cultures were incubated for 1 h at 37° C. in 5% CO₂. Then, the monolayer was washed five times with sterile PBS, fixed with methanol, Gram stained and examined microscopically. The adherence was measured as the number of lactobacilli adhering to the cells in 20 random microscopic fields. The assay was performed by triplicate.

In this study, the strain tested was strongly adhesive to Caco-2 and HT-29 epithelial cells since it showed means (+SD) of 897.2 (+190.1) and 352.7 (+64.5) adherent lactobacilli in 20 random microscopic fields, respectively. In comparison the mean values for L. salivarius CECT9145, a control strain with a high adherence to Caco-2, and HT-29 cells, were 904.4 (±229.7) and 342.9±(69.4), respectively.

### 4.3. Adhesion to Mucin

The adhesion of the strain to mucin was determined according to the method described by Cohen and Laux [13], with some modifications. Briefly, 100 μl of a solution (1 mg/ml) of porcine mucin (Sigma) in HEPES-buffered Hanks salt solution (HH) were immobilized in polystyrene microtiter plates (Maxisorp; Nunc, Roskilde, Denmark) after overnight incubation at 4° C. The wells were washed twice with 250 μl of HH. Parallel, the strain was grown overnight at 37° C. in MRS broth and the bacterial pellets from 1 ml fractions were obtained by centrifugation and washed with HH. Then, 10 μl of 10 mM carboxyfluorescein (Sigma) were added to the pellets and the bacterial suspensions were incubated for 20 min at 37° C. Subsequently, the bacterial cells were washed 3 times with HH and, finally, resuspended in 1 ml of HH. Then, a suspension of 50 μl of the fluorescent-labelled bacteria (~5×10⁷ CFU) was added to each well. After incubation for 1 h at 37° C., the plates were washed twice with 250 μl of HH to remove unattached cells, and incubated for 1 h at 60° C. in the presence of 50 μl of 1% sodium dodecyl sulphate (SDS)-0.1 M NaOH to release and lyse bound microorganisms. Fluorescence was measured in a fluorescence microplate reader (Tecan Austria GMBH, Salzburg, Austria). Adhesion was assessed as the percentage of the fluorescence retained in the wells after the washing steps when compared to that present in the labelled bacterial aliquots originally added to the wells. The assays were performed in triplicate.

The strain showed a high ability to adhere to porcine mucin (12.24±1.37). These values are similar or higher to those reported for other L. salivarius strains.

### 4.3. Production of Riboflavin, Folate and Cyanocobalamin

The riboflavin, folate and cyanocobalamin production by strain MP98 was determined using the microbiological assays described by Juarez del Valle et al. [14], Laino et al. [15], and Horwitz [16], respectively. Lactobacillus rhamnosus ATCC 7469, L. rhamnosus NCIMB 10463 and 7469, and Lactobacillus delbrueckii B12 were used as the indicator organism for the biosynthesis of the respective vitamins. The riboflavin production in the riboflavin-free medium was confirmed by an HPLC analysis following a procedure described previously [14]. The assays were performed in triplicate.

The strain produced neither folates nor cyanocobalamin under the assayed conditions. In contrast, it was able to produce riboflavin at a total concentration of ~210 ng/ml (intracellular riboflavin: 170.00±3.46 ng/ml; extracellular riboflavin: 39.97±2.97 ng/mL).

### Example 5. Characterization: Specific Properties for Gynecologycal Applications

### 5.1. Adherence to Vaginal Epithelial Cells

Adherence to vaginal epithelial cells collected from healthy premenopausal women was performed as described previously [17]. The adherence was measured as the number of lactobacilli adhering to the cells in 20 random microscopic fields. The assay was performed by triplicate. L. salivarius CECT9145 was used as a control strain due to its high adherence to this type of cells [18]. These assays were performed in triplicate.

In this study, the strain tested was strongly adhesive to vaginal cells since it showed a mean (±SD) of 343 (±51) adherent lactobacilli in 20 random microscopic fields. In comparison the mean value for L. salivarius CECT9145, a control strain with a high adherence to vaginal cells, was 333 (+56).

### 5.2. Antimicrobial Activity of MP98 Against Vaginal Pathogens

The antimicrobial activity of the strain against vaginal pathogens was performed using an overlay method [18]. The indicator strains included three strains of Candida albicans, two of Candida glabrata, two of Candida parapsilosis, three of Streptococcus agalactiae, five of G. vaginalis, and two of Ureaplasma urealyticum. These strains were isolated from cases of vaginal or cervico-vaginal infections (Complutense University of Madrid collection). These assays were performed in triplicate and the values are expressed as the mean±SD.

L. salivarius MP98 showed inhibitory antimicrobial activity (inhibition zone>2 mm around the streak) against all the G. vaginalis, Streptococcus agalactiae, Candida albicans, Candida glabrata, Candida parapsilosis and Ureaplasma urealyticum strains used as indicators in this study (Table 7).

TABLE 7
Antimicrobial activity and co-aggregation^{a} of
L. salivarius MP98 with vaginal pathogens.

| Strain | Inhibition zone (mm) | Co-aggregation |
|---|---|---|
| G. vaginalis MP14 | 4.4 | ++ |
| G. vaginalis MP17 | 4.5 | ++ |
| G. vaginalis MP20 | 4.4 | ++ |
| G. vaginalis MP24 | 4.3 | ++ |
| G. vaginalis MP29 | 4.2 | ++ |
| S. agalactiae MP07 | 2.3 | + |
| S. agalactiae MP12 | 2.4 | + |
| S. agalactiae MP46 | 2.1 | + |
| C. albicans MP09 | 3.3 | ++ |
| C. albicans MP18 | 3.7 | ++ |
| C. albicans MP31 | 3.1 | ++ |
| C. glabrata MP33 | 2.7 | ++ |
| C. glabrata MP37 | 2.7 | ++ |
| C. parapsilosis MP36 | 2.8 | ++ |
| C. parapsilosis MP48 | 2.9 | ++ |
| U. urealyticum MP39, | 3.3 | + |
| U. urealyticum MP57 | 3.2 | + |

^{a}Co-agregation was defined as the presence of large and dense visible clumps of bacteria (++), small and sparsely distributed clumps (+), or no visible clumps or bound bacteria (−).

### 5.3. Coaggregation Activity of MP98 Against Vaginal Pathogens

The capability of L. salivarius MP98 to aggregate with the vaginal pathogens cited above (section 5.2) was assessed as described previously [19]. These assays were performed in triplicate.

The strain was able to co-aggregate with all the tested vaginal pathogens. Co-aggregation was particularly intense with G. vaginalis and Candida strains (Table 7).

### 5.4. α-Amylase Activity of L. salivarius MP98

The α-amylase activity of L. salivarius MP98 was studied because of the beneficial implications of this enzyme for strains aimed to vaginal applications. Initially, this activity was qualitatively assessed using the procedure of Padmavathi et al. [20]. Later, the cell-bound α-amylase activity of L. salivarius MP98 was quantified with a specific kit (Kikkoman Co., Tokyo, Japan) as described previously [21]. These assays were performed in triplicate and the values are expressed as the mean±SD.

L. salivarius MP98 produced a zone of clearance around the colonies (1.9-2.1 mm) when the growth medium was flooded with iodine solution, indicating amylase production. Subsequently, a high level of α-amylase activity was found (0.80-0.84 U/mL) at 16 h (concentration of L. salivarius MP98: 8.5-8.9 log₁₀ CFU/mL) for, at least, 48 h.

### Example 6. Characterization: Specific Properties for Urological Applications

### 6.1. Antimicrobial Activity of MP98 Against Uropathogens

The antimicrobial activity of the strain against uropathogens was performed using an overlay method [18]. The indicator strains included Escherichia coli OBR-01, Klebsiella pneumoniae K12-4, Klebsiella pneumoniae Kp5, Enterococcus faecalis OEA1 and Enterococcus faecium EIPO. These strains were isolated from clinical cases of urinary tract infection (UTI) (Complutense University of Madrid collection). These assays were performed in triplicate and the values are expressed as the mean±SD.

L. salivarius MP98 showed inhibitory antimicrobial activity (inhibition zone ≥2 mm around the streak) against all uropathogenic strains used as indicators in this study. The sizes of the inhibition halos were >3 mm for E. coli OBR-01, E. faecalis OEA1 and E. faecium EIPO, and >5 mm for K. pneumoniae Kp5. The antimicrobial activity of L. salivarius MP98 was bigger than that displayed by Lacticaseibacillus rhamnosus, Limosilactobacillus reuteri, Lactococcus lactis and Streptococcus thermophilus strains isolated from commercial probiotic products specifically targeting UTIs.

### 6.2. Adherence of MP98 to Bladder Epithelial Cells

### 6.2.1. Strains and Growing Conditions

In addition to L. salivarius MP98, two other strains were included in this work: (a) L. plantarum CLC17, a strain previously isolated from milk but with negligible activity against uropathogenic bacteria and with almost no ability to adhere to gut epithelial cells or mucins [22] and, therefore, with not expected beneficial effect on UTIs; and (b) L. acidophilus 01, a strain isolated from a commercial supplement that claims benefits against UTIs. All strains were kept frozen at −70° C. in a sterilized mixture of culture medium (MRS broth; Pronadisa, Madrid, Spain) and glycerol (80:20, v/v). Inocula of the three strains (~10⁸ CFU mL⁻¹) were prepared by growing overnight the contents of thawed cryovials in MRS broth at 30° C. Bacteria were harvested by centrifugation (10,000×g, 10 min, 4° C.) and resuspended in Dulbecco's phosphate-buffered saline solution (DPBS, Lonza Walkersville, Inc., USA).

Two uropathogenic Escherichia coli (UPEC) strains were used for the adherence studies: E. coli ATCC 53503, a strain that expresses P fimbriae [23], and E. coli 10791 DSM 10791, a strain that harbors the fimH, papGII/III, focG, sfaS and hlyA genes [24]. In addition, three Gram-positive UTI clinical isolates from the Collection of the Complutense University of Madrid (Spain) were also included: Staphylococcus epidermidis 08-3, Enterococcus faecalis 04-1 and Enterococcus faecalis 08-1. E. coli ATCC 53503 and the strains of S. epidermidis and E. faecalis were grown in Tryptic Soy Broth (TSB; Scharlau, Barcelona, Spain), whereas E. coli 10791 was grown in Luria Broth (LB; Laboratories Conda Madrid, Spain). The contents of thawed cryovials were added to the medium and grown overnight at 37° C. Overnight cultures were harvested by centrifugation (10 000 g, 10 min, 4° C.) and resuspended in DPBS at a concentration of ~10⁸ CFU mL⁻¹.

### 6.2.2. Cell Cultures

T24 cells (ATCC HTB4) were used in this study since they are similar to primary human bladder epithelial cells [25]. T24 bladder cells were grown and maintained in McCoy's 5A medium (Sigma-Aldrich, St. Louis, MO, USA) supplemented with 10% (v/v) fetal bovine serum at 37° C. and antibiotics (100 IU/mL penicillin and 100 μg/mL streptomycin) in an atmosphere of 5% CO₂/95% air at constant humidity as previously described [26]. Cells were seeded in 24-well tissue plates and grown for 24 h to enable cell attachment and to obtain a cell monolayer.

### 6.2.3. Bacterial Adherence Assay

First, DPBS bacteria suspensions (~10⁸ CFU mL⁻¹) were prepared from overnight cultures. Then, confluent T24 cell monolayers (5×10⁵ cells/well) were washed with DPBS to eliminate antibiotic residues and overlaid with 0.5 mL of either a bacterial suspension or DPBS (as the control medium). After 1 h at 37° C. in a 5% CO₂ atmosphere, wells were gently washed with DPBS to remove unbound bacteria. Cells and adhered bacteria were then detached using a 0.05% trypsin-EDTA solution and sonicated in an ultrasonic sonication bath (three pulses, 10 s on, 3 s off) at 40 kHz to recover bacteria associated with cells. Bacterial counts (CFU mL⁻¹) were carried on TSA plates for E. coli strains, Enterococcus Selective Agar plates (Laboratories Conda) for enterococci, Manitol Agar plates (Laboratories Conda) for staphylococci and MRS plates for lactobacilli, as previously described [26]. The adherence percentage (%) was calculated as the number of adhered bacteria (CFU mL⁻¹) relative to the total number of bacteria added initially multiplied by 100.

### 6.2.4. Statistical Analysis

A paired sample t-test was conducted to evaluate whether pathogen adherence (%) in presence of Lactobacillus was different (P≤0.01) from pathogen adherence in his absence (control). Also, one-way analysis of variance post hoc comparison using the Tukey HSD test (P≤0.01) was used for the comparison of the values of inhibition (%) among those Lactobacillus stains that significantly decreased the % of adherence in respect to the control. The IBM SPSS program (v.22) for Windows was used for data processing.

### 6.2.5. Results of the Bacterial Adherence Assay

Both UPEC strains showed similar percentages of adherence to bladder cells (~9.0%-9.8%) while those displayed by the staphylococcal, enterococcal and lactobacilli strains were slightly higher (~10.5%-13%). Among them, L. salivarius MP98 and L. acidophilus 01 were the strains that showed the highest adherence ability in this assay (12.4 and 13%, respectively).

### 6.3. Activity of MP98 Against Uropathogens: Exclusion, Competition and Displacement Assays

For the exclusion assay, the L. salivarius MP98 suspension (~10⁸ CFU mL⁻¹) was first added to the cell monolayers and, after 1 h, non-bound bacteria were removed. Then, the uropathogen suspension (~10⁸ CFU mL⁻¹) was added to the wells and the mixture was incubated for a further 1 h. After this time, T24 cells and adhered bacteria (uropathogens/lactobacilli) were detached and uropathogen counting was carried out as described above.

For the displacement assay, the same experimental protocol was carried out, but adding first the uropathogen suspension (~10⁸ CFU mL⁻¹) and later, the L. salivarius MP98 suspension.

To test the ability of the Lactobacillus strains to compete with the uropathogens for adherence to T24 cells, both potential competitors (Lactobacillus strain and uropathogenic strain; ~10⁸ CFU mL⁻¹, each) were simultaneously added to the cells followed by incubation for 1 h. Non-bound bacteria were removed and the bacterial counts were carried out as described above.

The inhibition of the adherence of uropathogens was expressed as a percentage using the following formula: Inhibition (%)=100×(1−T1/T2), where T1 and T2 are the adherence percentage of uropathogens to T24 cells in the presence and absence of lactobacilli, respectively. All cell culture assays were performed in duplicate and three independent experiments were carried out.

The results obtained in these assays are shown in Table 8. The exclusion assay estimated the capacity of the lactobacilli to prevent UTIs (preventive action). The MP98 strain significantly decreased the adherence of the five uropathogenic strains to T24 cells (P<0.01). The inhibition of uropathogens adhesion ranged between 22.2% and 37.8%.

TABLE 8
Adherence (%) of uropathogenic strains to T24 epithelial
bladder cells as affected by L. salivarius MP98 in
exclusion, competition and displacement assays.

| | | | Uropathogen + | |
|---|---|---|---|---|
| | | Uropathogen | L. salivarius | |
| | Uropathogenic | (alone) | MP98 | P |
| | strain | Mean ± SD | Mean ± SD | value^{a} |
| Exclusion | E. coli ATCC | 9.82 ± 1.47 | 7.41 ± 0.62 | 0.010 |
| assay | 53503 | | | |
| | E. coli DSM | 9.01 ± 1.56 | 6.01 ± 0.80 | 0.001 |
| | 10791 | | | |
| | E. faecalis 04-1 | 11.3 ± 1.6 | 7.54 ± 1.24 | 0.003 |
| | E. faecalis 08-1 | 11.2 ± 1.2 | 8.03 ± 0.69 | 0.001 |
| | S. epidermidis 08-3 | 10.9 ± 1.9 | 6.78 ± 1.04 | 0.004 |
| Competi- | E. coli ATCC | 9.82 ± 1.47 | 7.29 ± 1.02 | 0.010 |
| tion | 53503 | | | |
| assay | E. coli DSM | 9.01 ± 1.56 | 6.19 ± 1.11 | 0.004 |
| | 10791 | | | |
| | E. faecalis 04-1 | 11.3 ± 1.6 | 7.40 ± 0.77 | 0.001 |
| | E. faecalis 08-1 | 11.2 ± 1.2 | 6.84 ± 0.97 | 0.000 |
| | S. epidermidis 08-3 | 10.9 ± 1.9 | 6.91 ± 0.95 | 0.000 |
| Dis- | E. coli ATCC | 9.82 ± 1.47 | 6.43 ± 0.85 | 0.002 |
| placement | 53503 | | | |
| assay | E. coli DSM | 9.01 ± 1.56 | 6.13 ± 0.89 | 0.005 |
| | 10791 | | | |
| | E. faecalis 04-1 | 11.3 ± 1.6 | 7.09 ± 1.12 | 0.002 |
| | E. faecalis 08-1 | 11.2 ± 1.2 | 6.30 ± 0.92 | 0.000 |
| | S. epidermidis 08-3 | 10.9 ± 1.9 | 6.90 ± 1.13 | 0.001 |

^{a}Different from the control (no MP98 added) for the corresponding uropathogenic strain.

The competition assay estimated of the capacity of the strain to avoid recurrent UTIs (prophylactic action). The adherence of the five uropathogenic strains to T24 cells significantly decreased when they were in the presence of strain MP98 (P<0.01). The inhibition of uropathogens adhesion ranged between 27.6% and 38.7%

Finally, the displacement assay was design to estimate the capacity of the lactobacilli to attenuate UTIs (treatment action). L. salivarius MP98 significantly (P<0.01) reduced the adherence of the five uropathogenic strains to the T24 cells. The inhibition of the adhesion of the different tested uropathogens ranged between 32.0% and 43.9%.

UTI and recurrent UTI are extremely common health problems, with an often unpredictable history, and usually, cause a strong discomfort among affected people. Thus, management and prevention of this condition is of utmost significance. The increasing resistance of uropathogens to antibiotics has prompted to reconsider empirical treatment of UTIs using antibiotics and, in this context, identifying new and effective strategies are a high priority in this field. One promising alternative is the use of probiotics in to replace uropathogens and restore a physiological urinary tract microbiota.

These results indicate that strain MP98 can block the adhesion of uropathogens (exclusion assay), can compete with pathogens for specific cell receptors (competition assay) and/or can provoke detachment of the uropathogens (displacement assay) from epithelial bladder cells. Adherence of pathogens to uroepithelial cell receptors seems to be the main step in the pathogenesis of UTIs; as a consequence, inhibition of uropathogens adherence to human bladder epithelial cells may be a key mechanism by which some this strain may play a role in preventing or treating such condition.

In this work, the methodology used for measuring uropathogen adhesion had been previously optimized and successfully applied to the study of the anti-adherence capacity of several phenolic metabolites against UPEC. In addition to classical UPEC strains, some UTI-related staphylococcal and enterococcal strains were also included in this study. Such Gram-positive bacteria are common inhabitants of the human urinary tract but, on the other hand, they can also be important uropathogens, particularly among elderly population, pregnant woman or people with any other risk factor for UTIs; however, they are often underrated as UTI etiological agents.

### Example 7. Characterization: Specific Properties for Respiratory Applications

### 7.1. Antimicrobial Activity of L. salivarius MP98 Against Respiratory Tract Pathogens

An overlay method was used as previously described to determine the ability of strain MP98 to inhibit the growth of a spectrum of bacterial strains previously isolated from clinical cases of acute otitis media (AOM) or bronchiolitis (Complutense University of Madrid collection) including: Alloiococcus otitidis MP02, Streptococcus faecalis MP64, pneumoniae MP07, Streptococcus pyogenes MP03, Enterococcus Staphylococcus aureus MP29, Staphylococcus epidermidis MP33, Haemophilus influenzae MP04, Moraxella catarrhalis MP08, Pseudomonas aeruginosa MP24 and E. coli MP69. Brain Heart Infusion (BHI, Oxoid, Basingstoke, UK), Columbia Nalidixic Acid (CNA, Biomerieux, Marcy-l′Étoile, France) or Trypticase Soy (TSA, Oxoid) agar plates (depending on the indicator strain) were overlaid with bacterial indicators, incubated at 37° C. for 48 h, and they were examined for zones of inhibition around the MP98 streaks.

L. salivarius MP98 showed a clear antimicrobial activity (inhibition zone >2 mm around the streak) against most of the respiratory-related indicator organisms used in this study (Table 9).

TABLE 9
Antimicrobial activity of L. salivarius MP98 against
bacterial strains isolated from clinical respiratory
cases as assessed by different assays.

| | | Co-cultures (CFU/mL) | |
|---|---|---|---|
| Indicator strain | Overlay method (cm) | Initial load | Final load |
| S. pneumoniae MP07 | 3.7 | 7.40 | nd |
| S. pyogenes MP03 | 2.1 | 7.74 | nd |
| S. aureus MP29 | 1.7 | 7.52 | 4.72 |
| S. epidermidis MP33 | 2.2 | 7.53 | 4.99 |
| A. otitidis MP02 | 1.2 | — | — |
| E. faecalis MP64 | 0.6 | 8.02 | 7.78 |
| H. influenzae MP04 | 2.1 | — | — |
| M. catarrhalis MP08 | 2.2 | — | — |
| P. aeruginosa MP24 | 1.7 | — | — |
| E. coli MP69 | 1.3 | 7.70 | 5.47 |

Nd, no growth detected; —, not assayed.

### 7.2. Coagregation Assays

The ability of the strain to aggregate with cells of the respiratory-related strains cited above was investigated following the procedure of Younes et al. [19].

L. salivarius MP98 showed a high potential for co-aggregating with bacterial strains involved in respiratory infections, particularly with those belonging to the genera Streptococcus, Alloiococcus, Enterococcus and Staphylococcus.

### 7.3. Co-Culture Studies

Broth co-cultures of strain MP98 and some of the respiratory-related strains cited above were performed in a BHI broth, since it was observed that this medium allowed most of their growth. The tubes were initially inoculated at a concentration of ~1×10⁸ CFU/mL for each of the bacterial strains (MP98 and the corresponding respiratory-related strain) and incubated overnight at 37° C. in aerobic conditions. BHI monocultures of each of the strains used in these assays were performed as control cultures. After incubation, samples of all the co-cultures and monocultures were seeded onto MRS, CNA, TSA and BHI agar plates for a selective enumeration based on the ability of the strains to growth and to display differential colony morphologies when inoculated on such media.

The results of the co-culture assays showed that most of the respiratory-related pathogens could not be detected, or their concentrations decreased notably after their overnight co-culture with the strain MP98 in the BHI broth (Table 9).

### Example 8. Characterization: Specific Properties for Prevention of Gout

### 8.1. Biochemistry of Uric Acid (UA)

Uric acid (UA; 7,9-dihydro-1H-purine-2,6,8(3H)-trione) is a heterocyclic organic compound with a molecular weight of 168 Da and the general formula C₅H₄N₄O₃. UA is a weak dibasic acid with dual pKa of 5.8 and 10.3, although in the human body under physiologic conditions (pH 7.4 and 37° C.) it is mostly present in the monodeprotonated ionic form. The divalent urate anion is almost absent in the human body because of the high second pKa, and in the biomedical literature the term urate ion is used to refer to the monovalent urate. Furthermore, the terms UA and urate are often used interchangeably and refer to the total pool of UA (dissociated and undissociated forms) because the ratio urate/UA in blood remains constant.

UA is normally found in the human body as a product of endogenous purine metabolism, being produced in kidneys, liver, intestine, vascular endothelium, and other muscle tissue. In addition, it also can have an exogenous origin, linked to the diet. The normal concentration 4 of UA in human blood ranges from 1.5 to 6.0 mg/dl in women, and from 2.5 to 7.0 mg/dl in men. This difference has been linked to the uricosuric effect of oestrogens in women.

UA concentration in humans is generally close to its solubility limit of 6.8 mg/dL; once the saturation limit is reached, it precipitates as monosodium urate (MSU) crystals. Additionally, different factors such as pH, temperature, ionic strength and binding to macromolecules (proteins and mucopolysaccharides) may modify its solubility.

### 8.2 Formation of UA

UA is the end oxidation product of purine metabolism in humans. Purines and pyrimidines are heterocyclic aromatic organic compounds (amines) that are found in all living things. They are composed of a nitrogenous base: adenine and guanine for purines, and cytosine, uracil and thymine for pyrimidines. These compounds play a key role in the transfer of genetic information, energy storage (ATP and GTP), cell signaling (CAMP and cGMP) and as enzyme cofactors (NADH, NADPH, coenzyme A). The cellular pool of purines and pyrimidines is tightly maintained via two 5 complementary pathways: salvage (which uses preformed nitrogenous bases from nucleotide degradation) and de novo synthesis by a functional protein complex (known as purinosome for purine biosynthesis) when there is an increased demand (cell growth or division).

The degradation of purines involves the sequential action of several enzymes that starting from nucleotides eventually produces UA. Most ingested nucleic acids are degraded by pancreatic and intestinal enzymes to nucleotides and, then, into nucleosides. If nucleosides are not absorbed, they are further degraded. Eventually, xanthine is the point of convergence for the metabolism of purines. Adenosine 5′-monophosphate (AMP) is converted into inosine by two different ways. The phosphate group is removed from AMP by the enzyme 5′-nucleotidase to form adenosine and, then, the enzyme adenosine deaminase removes an amino group to form inosine. Alternatively, the enzyme AMP deaminase converts AMP into inosine 5′-monophosphate (IMP) by removing an amino group, and then a nucleotidase removes the phosphate group to convert IMP in inosine. Likewise, guanosine 5′-monophosphate (GMP) is converted to guanosine by a nucleotidase and, then, to guanine by purine nucleoside phosphorylase (PNP).

Guanine and inosine are converted to UA by xanthine oxidase (XO) in two consecutive reactions, although inosine should be first transformed in hypoxanthine by purine nucleotide phosphorylase. Then, both hypoxanthine and guanine are oxidized to xanthine which can be further oxidized to UA.

### 8.3. Hypeuricemia

Hyperuricemia is defined as a serum UA level higher than 7.0 mg/dl in men and 6.0 mg/dL in women, measured at 37° C. and neutral pH. Although there is a considerable regional variation, hyperuricemia is patent in all populations where it has been studied. A systematic review of epidemiological studies has revealed that the reported prevalence oscillates between 1% (Papua New Guinea) and 85% (Marshall Islands), being Asia the region with global higher prevalence. In Italy, the prevalence of hyperuricemia was between 7 and 12%; these values were similar to other European countries. In most of the cases (90%) it is a secondary hyperuricemia, as a consequence of disease or external factors that influences the synthesis of UA and/or reduces its renal excretion.

With evolution, humans have lost the uricase enzyme and, therefore, the lack of the ability to metabolize UA into different end-products. In fact, uricase gene is not lost in humans, but it is a “pseudo-gene” that when it is transcribed in mRNA does not give a functional protein. It appears that the selection of species with mutation in uricase gene begun prior of the Miocene (first geological epoch of the Neogene Period), where species presented a decreased level of enzymatic activities. The loss of uricase and the increase of UA brought an advantage resulting in the evolution of the species; however, the driving force is still not clear. Furthermore, UA levels have increased through years with the dietary availability of purine-rich foods.

Some factors have an important role on the development of hyperuricemia such as sex, age, genetics, race or ethnicity. Sex seems to be an important non-modifiable risk factor as females have lower serum UA level than males. The explanation behind this fact is the oestrogen's enhancement of renal tubular urate excretion leading to the reduced risk of hyperuricemia and incident gout in pre-menopausal females. However, growing evidence suggest a substantial increase of hyperuricemia also among women (Rho et al., 2011).

### 8.4. Causes

Different causes leads to hyperuricemia, being the most important the overproduction of UA, a dysfunction of its excretion by kidney disease, or both. However, there is a long-held association of hyperuricemia with some dietary factors. Among them, alcohol and high meat or seafood intake can be considered the most classical factors. Most recently an association with diets containing high-purine vegetables has also been reported. Other dietary factors that contribute to hyperuricemia besides purine-rich foods are fructose/sugar-sweetened beverages, dairy products, and coffee.

### 8.5. Consequences

The clinical spectrum associated to hyperuricemia ranges from the classic presentation of episodic and acute inflammation of the first metatarsophalangeal joint to tophaceous gout, chronic polyarticular arthritis, urate nephrolithiasis and interstitial nephropathy. Among them, the most known disease related to hyperuricemia is gout, an inflammatory arthritis triggered by the crystallization of UA within the joints when 12 the blood level of UA reaches its physiologic limit of solubility. Gout affects at least 1% of the population in Western countries.

Furthermore, UA may predict the development of metabolic syndrome, obesity and diabetes. Recently, different studies have defined hyperuricemia as a risk factor for diseases such as arteriosclerosis, cerebrovascular and cardiovascular disease, chronic kidney disease, and nephropathy in diabetic patients. This condition is commonly observed in patients with metabolic syndrome together with diabetes, stroke, and coronary heart disease. Patients with hyperuricemia were found to be at a significantly higher risk for both stroke incidence (relative risk 1.41) and mortality (relative risk 1.26) than controls with normal levels of UA. Increased UA levels were found to be associated with increased risk of both hemorrhagic and ischemic strokes.

It has been proved that abnormal UA level induces endothelial dysfunction and renal fibrosis. Some studies have suggested that UA has also a direct influence in the development of neurodegenerative diseases. In this case, low levels lead to the onset of degenerative diseases as Alzheimer's disease, Huntington's disease, Parkinson's disease, and multiple sclerosis.

### 8.6. Treatment

Treatments for patients affected by hyperuricemia are based on modulating the activity of key enzymes involved in metabolism and excretion of UA. There are two main classes: uricostatic drugs (e.g. allopurinol and febuxostat), the most frequently used, that through a competitive inhibition of XO (xhanthine oxidase) reduce UA production, and uricosuric drugs (e.g. sulphinpyrazone, probeneceid and benzbromarone) which increase urinary UA excretion by blocking renal tubular reabsorption of urate.

The number of patients in gout therapy achieving serum urate levels <6 mg/dl (<0.35 mmol/L) is in the range of 20-40% for allopurinol and 45-67% for febuxostat. Uricosuric drugs (URAT1 inhibitors) are usually used as an option when the patients are intolerant to allopurinol

### 8.7. In Vitro Characterization of Specific Gout-Preventing Properties of L. salivarius MP98

### 8.7.1. Preparation of Intracellular Extracts

Each bacterial strain was grown in 50 mL of a suitable growth medium at the appropriate conditions until they had reached the stationary phase (24-48 h). Cells were concentrated by centrifugation at 19.000×g for 10 minutes at 4° C. The bacterial pellet was washed twice with 50 ml of cold NaCl (0.85%, w/v), and washed cells were suspended in 1 mL of 50 mM sodium phosphate buffer, pH 7. Concentrated cells (0.5 mL) were disrupted by mixing with same amount of zirconia/silica beads (0.1 mm diameter; Sigma-Aldrich) and shaking in a Fast Prep FP120 (QBioGene, Irvine, Califorma, USA) at 5 m/s for 15 s. The disruption was carried out four times and the cell suspension was kept on ice between each cycle to avoid protein degradation. The resulting mixture was centrifuged to remove glass beads and cell fragments at 19.000 xg for 15 minutes at 4° C. The supernatant or cell-free extract (CFE) was used immediately or kept at −20° C. until use.

### 8.7.2. Guanosine and Inosine Quantification

Inosine and guanosine were simultaneously detected and quantified by High Performance Liquid Chromatography (HPLC) using the method described by Li et al. with slight modifications. Analysis was performed on a HPLC system consisting of a 1260 Infinity Quaternary LC (Agilent) equipped with the Zorbax SBC18 (5 μm, 4.6×250 mm) column (Agilent) and a diode array detector. Working stocks solutions of inosine and guanosine (Sigma) were prepared at 33.7 and 35.7 mg/100 mL, respectively, in 100 mM K3PO4 pH 7. The stock solutions were then cleaned and sterilized by passing the solution through a filter with (0.22 μm pore diameter) before being degassed by sonication. Purines were separated using isocratic elution with 0.1 μM NaClO4 and 0.187 M H3PO4 as the mobile phase at a flow rate of 0.8 mL/min. The measured retention times at 245 nm were 3.25 and 3.40 min for guanosine and inosine, respectively.

The quantification of inosine and guanosine present in the samples was done using a calibration curve prepared by injecting increasing amounts of the standard solution (5, 10, 15 and 20 μL) and measuring the peak area obtained. Some samples were spiked with stock solution in order to confirm the identity of the purines detected and quantified. Inosine and guanosine stock solutions were stored at different temperatures (−20° C., 4° C., 25° C.) for up to a week and no change was recorded. Therefore, stocks solutions were prepared and stored at −20° C. until use

### 8.7.3. Evaluation of the Capability of the Bacteria to Use Purines

Bacteria from frozen stocks (150 μL) were transferred into tubes containing fresh broth culture (between 6 and 15 mL) and incubated at 37° C. for 24-48 hours until reaching the stationary phase. Then, bacteria were recovered by centrifugation (19.000×g for 10 minutes at 4° C.) and washed twice with the same volume of saline solution (0.85% NaCl, w/v). After the last washing step, the bacteria were suspended in 2.25 mL of the inosine and guanosine standard solution prepared as described above; alternatively, the bacteria were suspended in 500 μL of 100 mM K3PO4 pH 7 and 250 μL of the bacterial suspension was mixed with 1.25 mL of the inosine and guanosine standard solution. The mixture of bacteria and purine solution was incubated at 37° C. for 60 or 120 minutes with shaking (120 rpm). Then, the suspension was centrifuged (19.000×g for 10 minutes at 4° C.) to eliminate the bacteria and 810 μL of the clear supernatant was mixed with 90 μL 0.1M HClO4. Finally, samples were filtered (0.22 μm) and the filtrate was recovered in appropriate test tubes to determine the concentration of guanosine and inosine by HPLC analysis. These samples were frozen and stored at −20° C. To determine the time evolution of the purine captation by selected bacteria, the mixtures of bacterial suspension (>100 CFU/mL) and guanosine and inosine stock solution were incubated for 0, 15, 30, 60, 90, and 120 min, and following the procedure described above. To evaluate the ability of selected bacteria to transform guanosine and/or inosine, washed bacterial suspensions were replaced by the equivalent amount of CFE. The mixture of CFE and guanosine and inosine stock solution was incubated for 120 min at 37° C., following the protocol described for the suspensions of whole bacterial cells.

### 8.7.4. Statistical Analysis

Bacterial counts were recorded as CFU/mL and transformed to decimal logarithmic values before analysis. Experimental results are presented as mean values and standard deviations. The relationship between guanosine and inosine uptake and the bacterial concentration was analyzed using the Pearson correlation coefficient (r). The analysis of variance was used to assess differences in the use of guanosine and inosine among strains and it was performed using StatGraphics Centurion XVII version 17.0.16 (StatPoint Technologies Inc., Warrenton, Virginia, USA) and Microsoft Excel of Microsoft Office Professional Plus 2013 (Microsoft Corporation, USA).

### 8.7.5. Results: Evaluation of the Capability of the Bacteria to Use Purines

The capability of the strain to use purines was tested after mixing a concentrated suspension of washed cells with a standard solution of inosine and guanosine (1.26 mM) in 100 mM phosphate buffer pH 7. There was a strong correlation between guanosine and inosine uptake capacity.

The screening was performed using two different concentrations (single [~10⁹ cfu] and double strength) of the strain that were incubated for 60 and 120 minutes in 1.26 mM of each inosine and guanosine (2.25 mL) in order to understand their purine uptake capacity. L. salivarius MP98 consumed all the guanosine and inosine present in the extracellular medium even with the lower amount of cells (single strength) and the shortest incubation time (60 min).

### Example 9. Clinical Assays

### 9.1. Efficacy of L. salivarius MP98 to Increase Fertility-Related Outcomes: A Pilot Clinical Trial

Probiotics have been postulated as a method to improve the outcomes of assisted-reproduction technologies (ARTs). In fact, empiric use of commercial probiotic products is increasingly prescribed as an adjuvant treatment for women with infertility of unknown cause, despite there are few scientific or clinical evidences to support its usefulness for this target. In this study, a Ligilactobacillus strain previously isolated from a woman with a long record of genitouninary tract health and a successful reproductive history was characterized for a wide variety of general and vaginal-related probiotic properties; subsequently, it was orally administered to women with a history of either repetitive abortion (implantation failure) or infertility of unknown origin in order to elucidate if it can improve fertility outcomes. Before the initiation of the trial, a variety of vaginal parameters (Nugent score, pH, microbiological and immunological profiles) were studied and the data compared with those obtained from healthy fertile women. At the end of the trial, the same paremeters were assessed amon the treated women.

### 9.1.1. Participants and Design of the Assay

A total of 54 women, aged 28-45, participated in this study. Volunteers were classified into 3 groups. All women in the RA group (n=20) had a history of recurrent miscarriage with three or more pregnancy losses during the first 12 weeks of pregnancy. All women of the INF group (n=20) had a history of infertility (inability to conceive) despite being the recipients of ART for at least three times, including two cycles, at least, of in vitro fertilization (IVF). Finally, the control group (n=14) included fertile women having at least two children after uncomplicated term pregnancies. None of the women of the RA and INF groups received ART during the whole period of the study. None of the RA group components were diagnosed of antiphospholipidic syndrome and, therefore, they did not receive either heparic and/or salicilic acid during the study. None of the participants had received hormonal therapy, antibiotics or probiotics in the 4 weeks previous to sampling.

At recruitment (within the first three days post-ovulation; day 0), two samples were collected: a vaginal swab specimen for in fresh determination of the Nugent score, and a cervicovaginal lavage (CVL) of the cervical os and the vaginal walls with 10 ml of sterile normal saline for all the other analysis. Aliquots (2 ml) of the CVL samples were used for culture-based and PCR analyses. The remaining volume of the CVL samples was centrifuged at 800×g for 10 minutes at 4° C. CVL supernatants were stored at −80° C. until the immunological analyses were performed. Demographic, anthropometric, and health data (including a past or present history of recurrent infections at different body locations and use of antibiotics) were recorded at recruitment. High use of antibiotics was defined as receiving ≥4 antibiotic treatments per year because of recurrent infections while a range between 0 and 2 annual treatments was considered as a low use of antibiotics.

Starting at day 0, women of the RA and INF groups consumed (oral route) a daily sachet with ~50 mg of freeze-dried probiotic (~9 log₁₀ CFU of L. salivarius MP98) for 6 months or until a diagnosis of pregnancy (whatever happened first). At that point, the same two samples described above were collected from each woman. After a diagnosis of pregnancy, oral administration of the probiotic strain was maintained until the 15th week of pregnancy. All the spontaneous pregnancies that occurred within the first year after day 0 were recorded in this study.

Probiotic-containing sachets were kept at 4-8° C. throughout the study. All volunteers were provided with diaries to record compliance with the study product intake. Minimum compliance rate (% of the total treatment doses) was set at 86%. In accordance with the Declaration of Helsinki, all volunteers gave written informed consent to the protocol, which had been approved (protocol 10/017-E) by the Ethical Committee of Clinical Research of Hospital Clínico San Carlos Madrid (Spain).

At each of the two study visits, the pH of the lateral vaginal wall was measured (Whatman pH paper, pH 3.8-5.5 and pH 6.0-8.1). Nugent scoring was performed as described previously [28]. Briefly, the swab material was transferred to a glass slide, heat fixed, and Gram stained. Gram-positive, Gram-negative, and Gram-variable bacterial morphotypes were quantified. A Nugent score of 0-3 was considered normal, 4-6 was considered intermediate, and 7-10 was considered consistent with bacterial vaginosis [28].

CVL samples collected during the trial were serially diluted and plated onto Columbia Nalidixic Acid (CNA), Gardnerella (GAR), CHROMagar StrepB (CHR) Mac Conkey (MCK), Mycoplasma (MYC) and Sabouraud Dextrose Chloramphenicol (SDC) agar plates (BioMerieux, Marcy l′Etoile, France) for selective isolation and quantification of the main cultivable non-Lactobacillus bacteria and yeasts that may be found in the vagina, including the agents most frequently involved in vaginal infections. They were also spreaded onto agar plates of MRS (Oxoid, Basingstoke, UK) supplemented with either L-cysteine (2.5 g/L) (MRS-C) or horse blood (5%) (MRS-B) for isolation of lactobacilli, including L. iners (MRS-B). All media were incubated for 48 h at 37° C. under aerobic conditions, with the exception of the MRS-C and MRS-B plates, which were incubated anaerobically (85% nitrogen, 10% hydrogen, 5% carbon dioxide) in an anaerobic workstation (DW Scientific, Shipley, UK) for up to 72 h. After incubation, the colonies were recorded and at least one representative of each colony morphology was selected from the agar plates. The isolates were identified by Matrix Assisted Laser Desorption Ionization-Time of Flight (MALDI-TOF) mass spectrometry (Bruker, Germany). When the identification by MALDI-TOF was not possible at the species level (particularly in the case of lactobacilli isolates), the identification was carried out by 16S ribosomal RNA (rRNA) gene sequencing as described by Mediano et al. [29].

Approximately 1 mL of each CVL sample was used for DNA extraction following a method described previously [30]. Extracted DNA was eluted in 22 μL of nuclease-free water and stored at −20° C. until further analysis. Purity and concentration of each extracted DNA was initially estimated using a NanoDrop 1000 spectrophotometer (NanoDrop Technologies, Inc., Rockland, USA). Negative controls (blanks) were processed in parallel. DNA samples were used for real-time quantitative PCR (qPCR) detection and quantification of L. salivarius DNA. Primers and conditions for quantification of L. salivarius DNA have been described previously [31]. The DNA concentration of all samples was adjusted to 5 ng μL⁻¹. A commercial real-time PCR thermocycler (CFX96™, Biorad Laboratories, Hercules, CA, USA) was used for all experiments. Standard curves using 1:10 DNA dilutions (ranging from 2 ng to 0.2 μg) from L. salivarius MP98 were used to calculate the concentrations of the unknown bacterial genomic targets. Threshold cycle (Ct) values between 15.29 and 20.07 were obtained for this range of L. salivarius DNA (R2=0.9915). The Ct values measured for DNA extracted from non-target species (Lactobacillus reuteri MP07 and Lactobacillus plantarum MP02; our own collection) were ≥39. These two control strains were selected because they belong to the L. salivarius taxonomically closest species [32]. All samples and standards were run in triplicate.

The concentrations of several soluble immune factors (IL1β, IL1ra, IL2, IL4, IL5, IL6, IL7, IL8, IL9, IL10, IL12, IL13, IL15, IL17, IL6, basic FGF, eotaxin, GCSF, GMCSF, IFNγ, MCP1, MIP1α, MIP1β, PDGF-BB, RANTES, TNFα, VEGF) in the CVL supernatants were determined by magnetic bead-based multiplex immunoassays, using a Bioplex 200 instrument (Bio-Rad, Hercules, CA) and the Bio-Plex Pro™ Human Cytokine 27-plex Assay (ref. M500KCAFOY, Bio-Rad). In parallel, the levels of TGF-β 1 and TGF-β 2 were measured by ELISA with the RayBio® Human TGF-β 1 and Human TGF-β 2 ELISA kits, respectively (RayBiotech, Norcross, GA, USA). All determinations were carried out following the manufacturer's protocols and standard curves were performed for each analyte.

Microbiological data were recorded as CFU/mL and transformed to logarithmic values before statistical analysis. The normality of data distribution was analyzed using the Shapiro-Wilks test. Then, the quantitative variables were expressed as means and 95% confidence intervals (CI) or standard deviations (SD) when normally distributed and as medians and interquartile ranges (IQR) if they did not follow a normal distribution. The qualitative values were presented as total number of events and percentages. One-way ANOVA tests were used to compare the means of the experimental groups and Scheffé post hoc tests were used to identify which pairs of means were statistically different. The effect of the probiotic intervention on several vaginal parameters in each group of women with reproductive failure was analyzed using one-way ANOVA repeated measures tests. The Fisher's exact probability test, or the Freeman-Halton extension of the Fisher exact probability test for a 2×3 contingency table, was used for comparison of proportions and frequencies. For non-parametric analyses, differences between groups were assessed using Kruskal-Wallis tests and Wilcoxon-Mann-Whitney tests to identify which pair of groups were different, with Bonferroni correction for multiple comparisons when indicated. Correlations between the 20-major relative abundant bacterial genera were visualized using R package qgraph. Statistical analysis and plotting were performed either using Statgraphics Centurion XVIII version 18.1.06 (Statgraphics Technologies, Inc., The Plains, VA, USA) or in the R environment (version 3.5.1; R-project, http://www.r-project.org) and ggplot2. Differences were considered statistically significant at p<0.05.

### 9.1.2. Demographic, Anthropometric and Clinical Characteristics of the Participants

The characteristics of the 54 women that participated in this study are presented in Table 10. The mean (95% CI) age in the control group was 34.6 years (33.5-35.8), while in those of repetitive abortions (RA) and with infertility of unknown origin (INF) was 39.5 (38.5-40.9) and 37.95 (36.92-38.98) years, respectively. Women in the control group were significantly younger than other participants (p<0.001; one-way ANOVA), but there were no differences in mean values of body weight and height between the three groups of women.

TABLE 10
Characteristics of the participants (N = 54) which included fertile
women (Control group), women with a history of repetitive abortion
(RA group), and women with infertility of unknown origin (INF group).

| | | Control | RA | INF | |
|---|---|---|---|---|---|
| | | n = 14 | n = 20 | n = 20 | p-value |
| Age (years) | Mean | 34.6 | 39.5 | 37.95 | <0.001^{#} |
| | (95% CI) | (33.5-35.8)^{a} | (38.51-40.49)^{b} | (36.92-38.98)^{b} | |
| | Range | [28.0-45.0] | [35.0-43.0] | [35.0-44.0] | |
| | [min-max] | | | | |
| Weight (kg) | Mean | 62.4 | 69.25 | 67.55 | 0.054^{#} |
| | (95% CI) | (59.7-65.0) | (66.05-72.45) | (64.66-70.44) | |
| | Range | [46.0-87.0] | [54.0-86.0] | [55.0-82.0] | |
| | [min-max] | | | | |
| Height (cm) | Mean | 166 | 167.30 | 167.80 | 0.624^{#} |
| | (95% CI) | (164-168) | (164.18-170.42) | (165.48-170.12) | |
| | Range | [156-175] | [154-189] | [161-182] | |
| | [min-max] | | | | |
| Regularity of the | Yes, n | 10 (71) | 9 (45) | 10 (50) | 0.225* |
| menstrual cycle | (%) | | | | |
| | No, n | 4 (29) | 11 (55) | 10 (50) | |
| | (%) | | | | |
| Duration menstrual | Mean | 28.0 | 27.35 | 27.58 | 0.752^{#} |
| cycle (days) | (95% CI) | (27.4-28.7) | (26.73-27.97) | (26.87-28.28) | |
| | Range | [25.0-32.5] | [24.0-30.0] | [24.0-30.0] | |
| | [min-max] | | | | |

History of infections

| | | | | | |
|---|---|---|---|---|---|
| Vaginal | n (%) | 2 (14) | 12 (60) | 7 (35) | 0.024* |
| Urinary tract | n (%) | 2 (14) | 12 (60) | 12 (60) | 0.013* |
| Otorhinolaryngology | n (%) | 3 (21) | 7 (35) | 11 (55) | 0.128* |
| Lower respiratory tract | n (%) | 2 (14) | 7 (35) | 7 (35) | 0.344* |
| Skin | n (%) | 1 (7) | 3 (15) | 4 (20) | 0.9992* |
| Gastrointestinal | n (%) | 0 (0) | 1 (5) | 1 (5) | 1.000 |

Antibiotic usage^{a}

| | | | | | |
|---|---|---|---|---|---|
| In infancy | n (%) | 4 (29) | 18 (90) | 14 (70) | <0.001* |
| In adulthood | n (%) | 4 (29) | 15 (75) | 16 (80) | 0.016* |

History of other conditions

| | | | | | |
|---|---|---|---|---|---|
| Allergies | n (%) | 2 (14) | 5 (25) | 4 (20) | 0.995* |
| Food intolerance | n (%) | 0 (0) | 7 (35) | 11 (55) | <0.001* |
| Thyroid disease | n (%) | 0 (0) | 5 (25) | 3 (15) | 0.125* |

RA, women with repetitive abortions; INF, women with infertility of unknown origin.
^{a}Antibiotic usage means ≥4 annual treatments due to recurrent infections.
^{#}One-way ANOVA tests were used to evaluate differences in mean values of women age, weight and height and duration of the menstrual cycle between groups. Values followed by different bold letters within the same row indicate statistically significant differences between groups according to Scheffé post hoc comparison tests.
*Freeman-Halton extension of the Fisher exact probability tests for a 2 × 3 contingency table were used to compute the (two-tailed) probability of obtaining a distribution of values of categorical variables (regularity of the menstrual cycle, history of infections, antibiotic usage and history of other conditions).

Interestingly, statistically significant differences were found between the control women and those in the other two groups regarding a history of recurrent vaginal and urinary tract infections (p=0.024 and p=0.013, respectively; Fisher exact probability tests) and the use of antibiotics both during infancy (p<0.001) and adulthood (p=0.016), which were higher in the last two groups (Table 10). In contrast, no differences were observed among the three groups in relation to the rates of skin, lower respiratory tract and gastrointestinal infections (Table 10).

### 9.1.3. Baseline Vaginal Health Parameters

The vaginal pH values of the control group (4.53; range 4.38-4.68) were statistically different from those of the two study groups: 5.74 (5.53-5.94) and 6.03 (5.88-6.18) for RA and INF, respectively (p=0.000; one-vay ANOVA). Similarly, the Nugent scores of the two study groups were significantly higher [6.55 (5.99-7.11) and 6.40 (5.90-6.90), respectively], than those from controls (1.79 (1.27-2.30); p<0.001; one-vay ANOVA) (Table 11). The CVL concentrations of the growth factors TGF-β 1, TFG-β 2 and VEFG of the control group were 4.83 (4.65-5.01) pg/mL, 3.22 (3.10-3.34) pg/mL, and 406.0 (322.0-490.0) pg/mL, respectively, while they appeared to be halved in both study groups (RA and INF), the differences being statistically significant (Table 11). No differences were observed among the three groups in relation to the remaining soluble immune factors analyzed in this work, which showed a high degree of interindividual variability (data not shown).

TABLE 11
Comparison of baseline vaginal parameters (pH, Nugent score, cytokines,
and microbiology) of the participants (N = 54) which included fertile
women (Control group), women with a history of repetitive abortion (RA
group), and women with infertility of unknown origin (INF group).

| | | Control | RA | INF | |
|---|---|---|---|---|---|
| | | n = 14 | n = 20 | n = 20 | p-value |
| pH | Mean | 4.53 | 5.74 | 6.03 | <0.001^{#} |
| | (95% CI) | (4.38-4.68)^{a} | (5.53-5.94)^{b} | (5.88-6.18)^{b} | |
| | Range | [4.20-5.00] | [4.70-6.40] | [4.90-6.30] | |
| | [min-max] | | | | |
| Nugent score | Mean | 1.79 | 6.55 | 6.40 | <0.001^{#} |
| | (95% CI) | (1.27-2.30)^{a} | (5.99-7.11)^{b} | (5.90-6.90)^{b} | |
| | Range | [0.00-4.00] | [4.00-8.00] | [4.00-8.00] | |
| | [min-max] | | | | |
| TGF-β1 (pg/mL) | Mean | 4.83 | 2.46 | 2.13 | <0.001^{#} |
| | (95% CI) | (4.65-5.01)^{a} | (2.19-2.72)^{b} | (1.98-2.27)^{b} | |
| | Range | [4.20-5.30] | [1.60-3.50] | [1.60-2.70] | |
| | [min-max] | | | | |
| TGF-β2 (pg/mL) | Mean | 3.22 | 1.50 | 1.35 | <0.001^{#} |
| | (95% CI) | (3.10-3.34)^{a} | (1.35-1.65)^{b} | (1.24-1.46)^{b} | |
| | Range | [2.70-3.70] | [1.00-2.10] | [0.90-1.80] | |
| | [min-max] | | | | |
| VEGF (pg/mL) | Mean | 406.0 | 258.20 | 182.95 | 0.010^{#} |
| | (95% CI) | (322.0-490.0)^{a} | (203.76-312.64)^{a} | (136.19-229.71)^{b} | |
| | Range | [1.4-929.0] | [99.0-479.0] | [69.0-433.0] | |
| | [min-max] | | | | |
| Lactobacilli | n (%) | 14 (100) | 12 (60) | 7 (35) | <0.001* |
| positive women | | | | | |
| Viable counts | Mean | 7.33 | 4.19 | 3.65 | <0.001^{#} |
| (log₁₀ CFU/mL)** | (95% CI) | (7.15-7.46)^{a} | (3.71-5.14)^{b} | (2.47-5.98)^{b} | |
| | Range | [6.80-7.70] | [2.10-5.20] | [2.00-5.30] | |
| | [min-max] | | | | |

RA, women with repetitive abortions; INF, women with infertility of unknown origin. TGF-β 1, transforming growth factor β 1; TGF-β 2, transforming growth factor β 2; VEGF, vascular endothelial growth factor.
^{#}One-way ANOVA tests were used to evaluate differences in mean values between groups. Values followed by different bold letters within the same row indicate statistically significant differences between groups according to Scheffé post hoc comparison tests.
*Freeman-Halton extension of the Fisher exact probability tests for a 2 × 3 contingency table were used to compute the (two-tailed) probability of obtaining a distribution of values of lactobacilli positive women.
**Mean (95% CI) and range [min-max] values in lactobacilli-positive women.

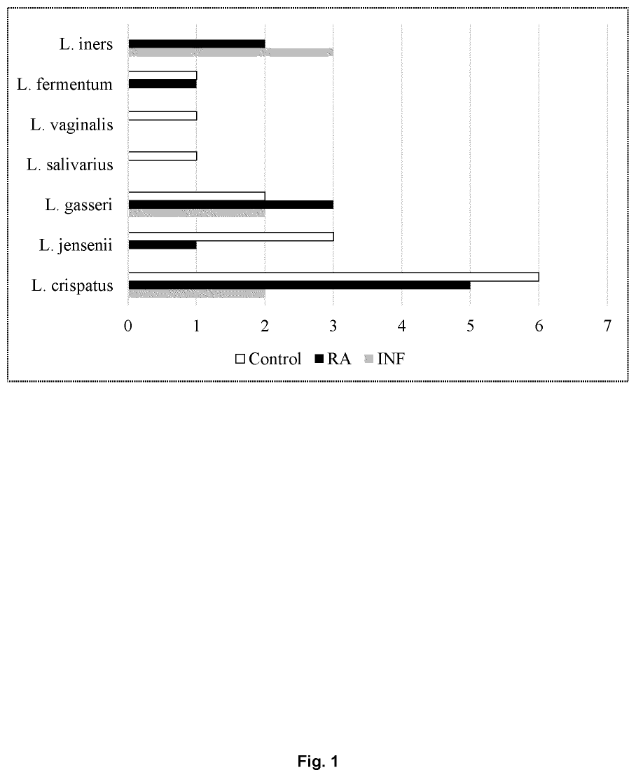

All women of the control group harbored lactobacilli in their vaginas (n=14), the mean (95% CI) value being 7.33 (7.15-7.46) log₁₀ CFU/mL using culture-dependent assessment. The frequency of lactobacilli detection was lower in the RA and INF groups: 60% and 35%, respectively (p<0.001; Fisher exact probability tests). In addition, mean lactobacilli concentrations were 2.62 and 2.75 log₁₀ units lower in CVL samples from lactobacilli-positive women in the RA and INF groups, respectively. The lactobacilli profile was also different (FIG. 1). Six species were identified in the samples from women of the control group, including L. crispatus (the dominant species), L. jensenii, L. gasseri, L. fermentum, L. salivarius, and L. vaginalis. However, the lactobacilli species profiles in the study groups (RA and INF) were narrower than in controls and, L. salivarius, and L. vaginalis were not detected. L. crispatus was the dominant species in 6 samples (43%) from fertile women, 5 samples (25%) from women with repetitive abortion and only 2 sample (10%) from infertile women. It is interesting to note that L. iners wasn't isolated from the control group while it was isolated from about one-third (5 out of a total of 19 lactobacilli positive samples) from samples of RA and INF groups. L. salivarius was detected in the sample of a unique woman from the control group as determined by species-specific qPCR (7.29 log₁₀ copies/mL) and culture (7.3 log₁₀ CFU/mL). The strain was genetically different from L. salivarius MP98 (results not shown).

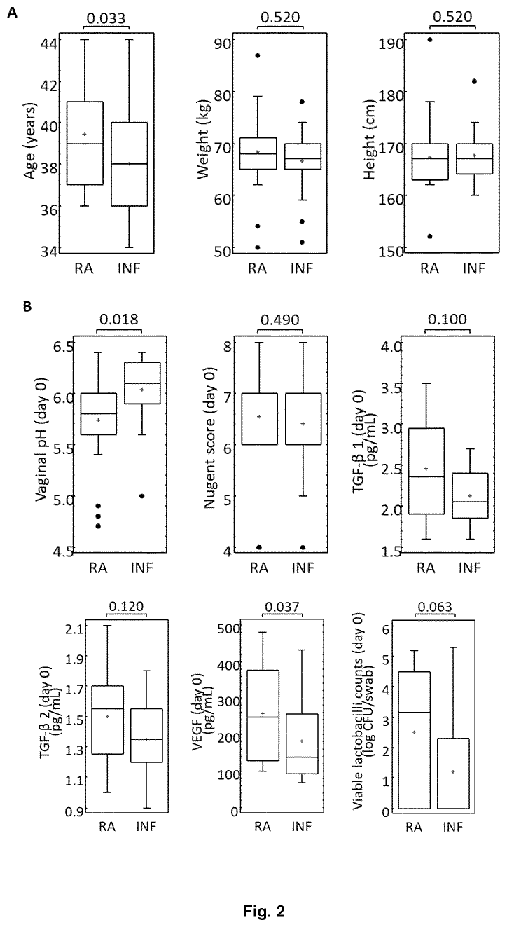

Globally, the comparison of RA and INF groups at the beginning of the study revealed some statistically relevant differences (FIG. 2). The mean of the vaginal pH values was 0.28 units higher in the INF group but the opposite was observed for VEGF, which had mean concentrations 75.25 μg/mL higher, respectively, in the RA group. No differences were observed regarding other characteristics, including age, weight, height, Nugent score, TGF-β 2, and lactobacilli viable counts (FIG. 2).

### 9.1.4. Main Outcome of the Trial: Pregnancies and Successful Pregnancies

Administration of L. salivarius MP98 (~9 log₁₀ CFU/day) for 6 months (or until a diagnosis of pregnancy if this happened first) to the women of the RA and INF groups led to 27 pregnacies out of the 40 participating patients. This means a pregnancy effectiveness of 67.5% with a 95% CI of 53-82% (Table 12). Among them, there were 25 succesfull pregnancies and 2 abortions. This means an effectiveness for reproductive success of 62.5% with a 95% CI of 48-78% (Table 12). Interestingly, all successful pregnancies led to full-term neonates (gestational age ≥38 weeks).

Women of the RA group had the highest rate of reproductive success (15 full term pregnancies and 1 abortion out of 20 participants) (Table 12). The rate in the INF group was lower although still noticeable: 11 pregnancies (10 full term and 1 abortions) out of 20 enrolled. Therefore, the pregnancy effectiveness and successful pregnancy rates (95% CI) tended to be higher in RA group that in INF group [RR (95% CI)=1.45 (0.92-2.29) and 1.50 (0.90-2.49), respectively], although the difference between both groups did not reach statistical significance (Table 12). It must be highlighted that all women of these groups had been unsuccessfully subjected to ART interventions in previous attempts to avoid spontaneous miscarriage (RA group) or to get pregnant (INF group).

TABLE 12
Main outcomes after the probiotic treatment with L. salivarius
MP98 in women with repetitive abortion (RA) and women
with infertility of unknown origin (INF).

| | | | | Ratio |
|---|---|---|---|---|
| | Group | | Total | (95% CI) |
| Outcome | RA | INF | (RA + INF) | (RA/INF) |
| Pregnancy | 16/20 | 11/20 | 27/40 | |
| (no. events/total | | | | |
| events) | | | | |
| Pregnancy | 80% | 55% | 67.5% | 1.45 |
| effectiveness | (62.47- | (33.20- | (52.98- | (0.92-2.29) |
| (95% CI) | 97.53%) | 76.80%) | 82.02%) | |
| Successful | 15/20 | 10/20 | 25/40 | |
| pregnancy* | | | | |
| (no. events/total | | | | |
| events) | | | | |
| Reproductive | 75% | 50% | 62.5% | 1.50 |
| success | (56.02- | (28.09- | (47.50- | (0.90-2.49) |
| (95% CI) | 93.98%) | 71.91%) | 77.50%) | |

*1 woman in each group ended up in abortion.

### 9.1.5. Secondary Outcomes Associated with the Probiotic Treatment: RA Group

There were no differences in age, weight or height between women in the RA group that ended up having a sucessful pregnancy (n=15) and those who did not (n=5) after the probiotic intervention. However, differential changes in their vaginal parameters were observed (Table 13): the vaginal pH of women who delivered was about 1.18 units lower than in those who did not (p<0.01; one-way ANOVA). Similar results were noted for the Nugent score [a mean (95% CI) reduction of 4.2 (4.68-3.26 units in women who got pregnant after the probiotic intervention versus a mean (95% CI) reduction of 1.2 (−1.43-−0.75) units in those who did not complete a full-term pregnancy; p=<0.01 one-way ANOVA) (Table 13).

TABLE 13
Effect of the probiotic intervention with L. salivarius MP98 on
the vaginal parameters of women who were able to complete a full-term
pregnancy (n = 15) and of those who did not (n = 5) among
the women that had a history of repetitive abortion (RA group; n = 20).

| | Probiotic intervention resulted in pregnancy | | |
|---|---|---|---|
| | Yes (n = 15) | No (n = 5) | p- |
| Vaginal parameter | [mean (95% CI)] | [mean (95% CI)] | value^{#} |

pH

| | | | |
|---|---|---|---|
| Baseline | 5.61 (5.37-5.84) | 6.16 (6.04-6.39) | 0.007 |
| Post-intervention | 4.44 (4.31-4.58) | 5.64 (5.43-6.06) | <0.001 |
| Change | −1.19 (−1.36-−0.86) | −0.52 (−0.62-−0.32) | <0.001 |
| p-value† | <0.001 | 0.025 | |

Nugent score

| | | | |
|---|---|---|---|
| Baseline | 6.31 (5.62-7.00) | 7.4 (7.12-7.95) | 0.074 |
| Post-intervention | 2.25 (1.72-2.78) | 6.2 (5.78-7.04) | 0.001 |
| Change | −4.2 (−4.68-−3.26) | −1.2 (−1.43-−0.75) | <0.001 |
| p-value† | <0.001 | 0.025 | |

TGF-β1 (pg/mL)

| | | | |
|---|---|---|---|
| Baseline | 2.64(2.36-2.91) | 1.78 (1.70-1.93) | 0.002 |
| Post-intervention | 4.18125 (3.94-4.42) | 2.18 (1.98-2.57) | 0.001 |
| Change | 1.59 (1.31-2.15) | 0.4 (0.26-0.67) | <0.001 |
| p-value† | <0.001 | 0.025 | |

TGF-β2 (pg/mL)

| | | | |
|---|---|---|---|
| Baseline | 1.59 (1.43-1.74) | 1.12 (0.98-1.39) | 0.007 |
| Post-intervention | 2.91 (2.69-3.13) | 1.34 (1.17-1.68) | 0.001 |
| Change | 1.36 (1.17-1.73) | 0.22 (0.03-0.59) | <0.001 |
| p-value† | <0.001 | 0.655 | |

VEGF (pg/mL)

| | | | |
|---|---|---|---|
| Baseline | 296.66 (239.09-354.16) | 106.60 (103.08-113.55) | 0.001 |
| Post-intervention | 586.88 (479.76-693.99) | 126.20 (113.63-151.04) | <0.001 |
| Change | 306.40 (233.76-449.93) | 19.6 (10.10-38.37) | <0.001 |
| p-value† | <0.001 | 0.025 | |

Lactobacilli presence [n (%)]

| | | | |
|---|---|---|---|
| Baseline | 10 (66.66) | 2 (40) | 0.172* |
| Post-intervention | 15 (100) | 3 (60) | 0.052* |
| Change | 5 (33.33) | 1 (20) | 0.613* |

Lactobacilli counts (log₁₀ CFU/mL)

| | | | |
|---|---|---|---|
| Initial | 4.08 (3.58-5.08) | 4.75 (4.50-5.24) | 0.685 |
| Final | 7.34 (7.08-7.85) | 4.23 (3.42-5.84) | <0.001 |
| Change | 3.12 (2.51-4.33) | 0.4 (0.33-0.54) | <0.001 |
| p-value† | <0.001 | 0.525 | |

L. salivarius qPCR [n (%)]

| | | | |
|---|---|---|---|
| Initial | nd | nd | — |
| Final | 15 (100) | 2 (40) | 0.035* |

L. salivarius qPCR (log₁₀ copies/mL)**

| | | | |
|---|---|---|---|
| Initial | — | — | — |
| Final | 7.81 (7.52-8.39) | 2.25 (1.50-3.73) | <0.001 |

^{#}One-way ANOVA tests were used to evaluate differences in mean values between groups, except for lactobacilli presence.
* Fisher exact probability test for a 2 × 2 contingency table.
†One-way repeated measures ANOVA tests were used to determine whether there was a change in each group of participants when comparing the baseline and post-intervention parameters.
**Mean (95% CI) of L. salivarius qPCR (copies/mL) in positive samples.

The vaginal cytokine concentrations also diferred in both subgroups of women (with successful pregnancy or not) in the RA group after the probiotic treatment. There was no modification in the vaginal TGF-β 1, TGF-β 2 and VEGF concentrations with respect to the baseline in the women who did not become pregnant, but there was a mean (95% CI) significant increase of 1.59 (1.31-2.15) pg/mL, 1.36 (1.17-1.73) pg/mL and 306.40 (233.76-449.93) pg/mL], respectively, in those who did (p<0.001; one-way repeated measures ANOVA) (Table 13). In addition, it should be noted that there were already differences in the concentration of these cytokines even before starting the treatment between those that became and those that did not become pregnant (Table 13).

On the other hand, the probiotic treatment resulted in a mean (95% CI) increase in lactobacilli counts of 3.12 (2.51-4.33) log₁₀ CFU/mL in women that finally got pregnant, but there were not significant changes in those that did not (Table 12). The presence of L. salivarius [mean (95% CI)=7.81 (7.52-8.39)) log₁₀ copies/mL] was confirmed by qPCR in all women that got pregnant, but only in 50% of the women with unsuccessful pregnancies and their concentration being significantly lower [mean (95% CI)=2.25 (1.50-3.73) copies/mL] (Table 13). The lactobacilli profile in CVL samples obtained at the beginning of the probiotic treatment and after 6 months or until a diagnosis of pregnancy is presented in Table 13b.

Table 13b Changes in the profile of dominant Lactobacillus especies in CVL samples from women with a history of repetitive abortion (RA group) and women with infertility of unknown origin (INF group) after the probiotic intervention with L. salivarius MP98. Their outcome is indicated in the last column: +, successful full-term pregnancy; A+, abortion; −, no pregancy. The presence of isolates from a given species is indicated by grey color without −, A+ or +symbol

The most noticeable difference was the presence of viable L. salivarius in most women (17/20) after the probiotic treatment.

### 9.1.6. Secondary Outcomes Associated with the Probiotic Treatment: INF Group

The women in the INF group that got pregnant after the probiotic intervention (n=10) and those who did not (n=10) did not differ in age, weight and height. The CVL pH and the Nugent score decreased significantly in all members of the INF group after the probiotic treatment (p<0.05; one-way repeated measures ANOVA), although the magnitude of the change was smaller in the women that did not get pregnant when compared to those that got pregnant (Table 14). Specifically, the mean (95% CI) reductions in CVL pH and Nugent score in women that got pregnant were −1.41 (−1.59-1.23) and −4.2 (−4.90-3.50), respectively, and in women that did not get pregnancy these reductions were only −0.28 (−0.43-0.13) and −0.9 (−1.44-0.36), respectively (Table 14).

The change in the vaginal cytokine concentrations after the probiotic treatment was similar to that described in the RA group; there was no modification in the vaginal TGF-β1, TGF-β2 and VEGF levels of women who did not become pregnant, but there was a mean (95% CI) significant increase of 2.32 (2.16-2.48) pg/mL, 1.35 (1.26-1.44) pg/mL and 472.3 (379.31-565.29) pg/mL, respectively, in those who did (Table 14). In this INF group, there were already differences in the concentrations of TGF-β2 and VEGF, but not in that of TGF-31, between those that became and those that did not became pregnant even before starting the treatment (Table 14).

The probiotic intervention resulted in a high degree of vaginal colonization by lactobacilli [6.70 (6.19-7.15) log₁₀ CFU/mL] of all women that got pregnant, while this only happened in 30% of those that experienced a treatment failure, the density of lactobacilli reached being significantly lower [5.4 (3.73-6.88) log₁₀ CFU/mL)] (Table 14). Similarly to the RA group, the presence of L. salivarius [mean (95% CI)=7.05 (6.39-7.63) copies/mL] was confirmed by qPCR in all women that got pregnant, but only in 10% of the women with unsuccessful pregnancies and, then, at a lower concentration (Table 14). The main difference in the lactobacilli profile of CVL samples of women in the INF group registered after the probiotic intervention was the detection of viable L. salivarius in all women who got pregnant, but only in 1 out of 10 of those women that failed to get pregnant.

TABLE 14
Effect of the probiotic intervention with L. salivarius MP98 on
the vaginal parameters of women who were able to complete a full-term
pregnancy (n = 10) and of those who did not (n = 10) among
the women with infertility of unknown origin (INF group; n = 20).

| | Probiotic intervention resulted in pregnancy | | |
|---|---|---|---|
| | Yes (n = 10) | No (n = 10) | |
| Vaginal parameter | [mean (95% CI)] | [mean (95% CI)] | p-value^{#} |

pH

| | | | |
|---|---|---|---|
| Baseline | 5.9 (5.68-6.12) | 6.16 (6.00-6.32) | 0.026 |
| Post-intervention | 4.49 (4.37-4.61) | 5.88 (5.69-6.07) | <0.001 |
| Change | −1.41 (−1.59-1.23) | −0.28 (−0.43-0.13) | <0.001 |
| p-value† | 0.001 | 0.002 | |

Nugent score

| | | | |
|---|---|---|---|
| Baseline | 6.2 (5.44-6.96) | 6.6 (5.93-7.27) | 0.571 |
| Post-intervention | 2 (1.42-2.58) | 5.7 (5.04-6.36) | <0.001 |
| Change | −4.2 (−4.90-3.50) | −0.9 (−1.44-0.36) | <0.001 |
| p-value† | 0.001 | 0.027 | |

TGF-β1 (pg/mL)

| | | | |
|---|---|---|---|
| Baseline | 2.26 (2.05-2.47) | 1.99 (1.82-2.16) | 0.070 |
| Post-intervention | 4.58 (4.39-4.77) | 2.23 (2.06-2.40) | <0.001 |
| Change | 2.32 (2.16-2.48) | 0.24 (0.09-0.39) | <0.001 |
| p-value† | 0.001 | 0.027 | |

TGF-β2 (pg/mL)

| | | | |
|---|---|---|---|
| Baseline | 1.51 (1.386-1.64) | 1.19 (1.09-1.29) | <0.001 |
| Post-intervention | 2.86 (2.72-3.00) | 1.33 (1.20-1.46) | <0.001 |
| Change | 1.35 (1.26-1.44) | 0.14 (0.06-0.22) | <0.001 |
| p-value† | 0.001 | 0.027 | |

VEGF (pg/mL)

| | | | |
|---|---|---|---|
| Baseline | 261.8 (203.52-320.08) | 104.1 (81.07-127.13) | <0.001 |
| Post-intervention | 734.1 (600.11-868.09) | 119.7 (89.24-150.16) | <0.001 |
| Change | 472.3 (379.31-565.29) | 15.6 (3.81-27.39) | <0.001 |
| p-value† | 0.001 | 0.057 | |

Lactobacilli presence [n (%)]

| | | | |
|---|---|---|---|
| Baseline | 4 (40) | 3 (30) | 1.000* |
| Post-intervention | 10 (100) | 3 (30) | 0.003* |
| Change | 6 (60) | 0 (0) | 0.020* |

Lactobacilli counts (log₁₀ CFU/mL)

| | | | |
|---|---|---|---|
| Initial | 3.26 (1.77-4.60) | 3.57 (1.81-5.12) | 0.290 |
| Final | 6.70 (6.19-7.15) | 5.4 (3.73-6.88) | 0.032 |
| Change | 3.23 (2.46-3.90) | 2.05 (0.05-3.82) | <0.001 |
| p-value† | <0.001 | 0.451 | |

L. salivarius qPCR [n (%)]

| | | | |
|---|---|---|---|
| Initial | nd | nd | — |
| Final | 10 (100) | 1 (10) | 0.002* |

L. salivarius qPCR (log₁₀ copies/mL)**

| | | | |
|---|---|---|---|
| Initial | — | — | — |
| Final | 7.05 (6.39-7.63) | 4.20 | — |

^{#}One-way ANOVA tests were used to evaluate differences in mean values between groups, except for lactobacilli presence.
*Fisher exact probability test for a 2 × 2 contingency table.
†One-way repeated measures ANOVA tests were used to determine whether there was a change in each group of participants when comparing the baseline and post-intervention parameters.
**Mean (95% CI) of L. salivarius qPCR (copies/mL) in positive samples.

### 9.1.7. Comparison of Vaginal Parameters Between Women Who Became Pregnant and Those Who Did not from Both the RA and INF Groups

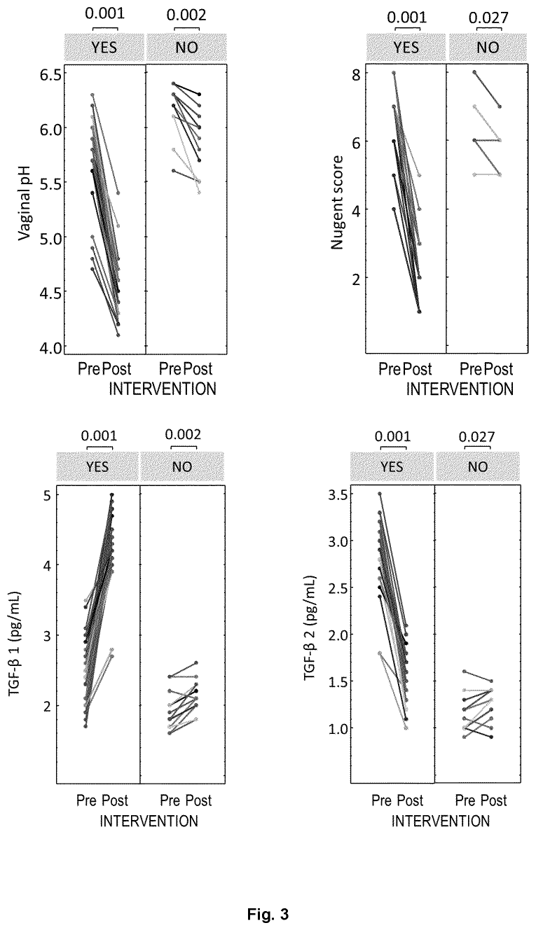

The mean (95% CI) pH value in CVL samples was sligthy but significantly more acidic in the women who become pregnant [5.72 (5.45-5.99) units] than in those who did not [6.16 (6.01-6.31) units] (p<0.001; one-way ANOVA) (FIG. 3). There were also differences in the concentration of vaginal cytokines TGF-β1, TGF-β2 and VEFG at the beginning of the study according to the final pregnancy outcome, but the differences were similar to those described already separately for RA and INF groups (FIG. 3). The only parameters that did not differed initially between both groups were the Nugent score and the frequency of detection and counts of lactobacilli (FIG. 3). Globally, Lactobacillus was detected in all women who became pregnant, but only in the 40% of those that did not (p<0.001; Fisher exact probability test).

The probiotic intervention resulted in differential and remarkable changes in the vaginal parameters in those women who became pregnant but not in those who did not (FIG. 3). First, the probiotic administration of L. salivarius MP98 resulted to be more effective regarding the change in the vaginal pH and Nugent score in women who got pregnant, which recorded mean (95% CI) decreases of −1.28 (−1.49-1.07) and 4.2 (−4.82-3.58) units, respectively (p<0.001; one-way repeated measures ANOVA). In contrast, the change in these two parameters was smaller [−0.36 (−0.51-0.21) and −1 (-1.47-0.53) units, respectively] in the group of women who did not get pregnant (FIG. 3). Second, the probiotic intervention led to a significant increase in the concentrations of vaginal cytokines TGF-β 1, TGF-β 2 and VEFG [mean (95% CI) increase of 1.884 (1.52-2.24) pg/mL, 1.36 (1.17-1.54) pg/mL and 372.76 (270.28-475.24) pg/mL, respectively] in women who got pregnant but no change was registered in the group that did not (FIG. 3). And, third, regarding the lactobacilli profile of CVL samples, there was a mean (95% CI) increase of 3.08 (2.50-4.21)) log₁₀ units in viable Lactobacillus counts after the probiotic treatment in the group of women who became pregnant as opposed to those that did not. Differences were also noted on the L. salivarius content in CVL samples. This lactobacilli species was detected, and at a high concentration [mean (95% CI)=7.51 (7.162-8.19) log₁₀ copies/mL], in CVL samples from all women having a succesful pregnancy unlike women who did not become pregnant (FIG. 3).

### 9.1.8. Comparison of Vaginal Parameters Between Control Women, all Women Who Became Pregnant and Those Who Did not from Both RA and INF Groups

The analysis of post-intervention vaginal parameters (pH, Nugent score, TGF-β 1, TGF-β 2, VEGF, lactobacilli counts) revealed that the pH value of CVL samples and Nugent score in women who became pregnant after the probiotic intervention were similar to those of fertile control women (Table 15).

The concentrations of TGF-β 1, TGF-β 2, and VEGF in post-intervention CVL samples of women who became pregnant were closer to those found in fertile control women, although statistically significant differences were found between them (Table 15). Besides, it is remarkable to note that the post-intervention concentration of VEGF in women that became pregnant was about 50% more than the registered in fertile control women [mean (95% CI)=662.72 (541.37-784.07) pg/mL and 406.0 (322.0-490.0) pg/mL, respectively]. There was a high interindividual variation in Lactobacilli counts varying from undetectable (in 60% of the women who did not become pregnant) to 6.40 log₁₀ CFU/mL in CVL samples of women who did not become pregnant after the probiotic intervention, but the mean (95% CI) value [4.82 (4.05-6.34) log₁₀ CFU/mL] was lower than in samples of the other participants] (Table 15). There was less than 1 log₁₀ CFU/mL difference between the lactobacilli viable counts in CVL samples of women who enjoyed a full term pregnancy after the probiotic intervention and those of fertile controls [mean (95% CI)=7.04 (6.74-7.64) log₁₀ CFU/mL and 7.24 (6.89-7.60) log₁₀ CFU/mL, respectively] (Table 15).

TABLE 15
Comparison of vaginal parameters (pH, Nugent score, TGF-β 1, TGF-β
2, and VEGF concentrations, and Lactobacillus counts) of all women who were
able to complete a full-term pregnancy (n = 25) and of those who did not
(n = 15) among all women with a history of repetitive abortion and with infertility
of unknown origin (RA and INF groups) after the probiotic intervention with
L. salivarius MP98 and vaginal parameters of fertile women (Control group; n = 14).

| | | Probiotic intervention resulted | | |
|---|---|---|---|---|
| | | in pregnancy | | |
| | Control (n = 14) | Yes (n = 25) | No (n = 15) | p- |
| | Mean (95% CI) | Mean (95% CI) | Mean (95% CI) | value |
| pH | 4.53 | 4.44 | 5.80 | <0.001^{#} |
| | (4.38-4.68)^{a} | (4.32-4.55)^{a} | (5.58-6.02)^{b} | |
| Nugent score | 1.79 | 2.04 | 5.87 | <0.001^{#} |
| | (1.27-2.30)^{a} | (1.55-2.53)^{a} | (5.25-6.48)^{b} | |
| TGF-β1 (pg/mL) | 4.83 | 4.40 | 2.21 | <0.001^{#} |
| | (4.65-5.01)^{a} | (4.20-4.59)^{b} | (2.02-2.40)^{c} | |
| TGF-β2 (pg/mL) | 3.22 | 2.94 | 1.33 | <0.001^{#} |
| | (3.10-3.34)^{a} | (2.77-3.10)^{b} | (1.18-1.49)^{c} | |
| VEGF (pg/mL) | 406.0 | 662.72 | 121.87 | <0.001^{#} |
| | (322.0-490.0)^{a} | (541.37-784.07)^{b} | (96.02-147.71)^{c} | |
| Lactobacilli Positive | 14 (100) | 25 (100) | 6 (40) | <0.001* |
| women [n (%)] | | | | |
| Viable counts | 7.24 | 7.04 | 4.82 | <0.001^{#} |
| (log₁₀ CFU/mL)** | (6.89-7.60)^{a} | (6.74-7.64)^{a} | (4.05-6.34)^{b} | |

TGF-β1, transforming growth factor-β 1; TGF-β 2, transforming growth factor-β2; VEGF, vascular endothelial growth factor.
^{#}One-way ANOVA tests were used to evaluate differences in mean values between groups. Values followed by different bold letters within the same row indicate statistically significant differences between groups according to Scheffé post hoc comparison tests.
*Freeman-Halton extension of the Fisher exact probability tests for a 2 × 3 contingency table were used to compute the (two-tailed) probability of obtaining a distribution of values of lactobacilli positive women.
**Mean (95% CI) values in lactobacilli-positive women.

### 9.1.9. Discussion

In this study, significant differences in the vaginal Lactobacillus population were detected between fertile women with a history of reproductive success and those with a history of reproductive failure, because of either recurrent spontaneous abortion or infertility. The lowest Nugent scores and vaginal pH values were closely linked to Lactobacillus-dominated communities while the contrary was associated with a depletion of lactobacilli. Similar findings have been described previously. Interestingly, the number of antibiotic treatments was significantly lower among women of the control group than among those of the RA and INF groups. Antibiotics have been reported as one of the main factors leading to a depletion of authoctonous vaginal lactobacilli. Our data suggest that, on the one hand, the impact of antibiotherapy on vaginal lactobacilli may impair fertility or embryo implantation and, on the other hand, that this effect may be overcome by probiotic modulation of the vaginal microbiota.

The in vitro assessment of some properties of L. salivarius MP98 showed that this strain was able to inhibit the growth of all the strains of G. vaginalis, S. agalactiae, C. albicans, C. glabrata, C. parapsilosis and U. urealyticum used as indicators in this work. Such activity may be important to reduce the risk of genitourinary tract infections, which have been linked to poor reproductive outcomes. L. salivarius MP98 displayed a noticeable α-amylase activity and a high acidifying activity as a result of its ability to produce high amounts of L-lactic acid. These properties are very relevant for vaginal homeostasis since they promote the existance of a highly acidic vaginal pH (≤4.5), which is a feature of a Lactobacillus-dominated healthy vaginal ecosystem.

L. salivarius MP98 was highly adhesive to vaginal epithelial cells and co-aggregated with the vaginal pathobionts tested in this study. These properties are also attractive for probiotics targeting the vagina since they allow a higher competitiveness and fitness in relation to other microorganisms that may inhabit or reach the vaginal cavity. In addition, L. salivarius MP98 showed a high rate of survival when exposed to conditions simlar to those found in the human digestive tract and was highly adhesive to intestinal epithelial cells. These properties are also relevant when a probiotic strain is going to be adninistrated per se as this was the intention in the subsequent trial.

In this work, L. salivarius MP98 intake led to noticeable pregnancy rates among women of the RA and INF groups, which involved significant changes in pH and Nugent score values, and in microbiological (Lactobacillus concentration, presence of L. salivarius cells and DNA) and immunological (VEGF, TGF-β 1 and TGF-β 2) changes in the vaginal ecosystem.

The changes in the concentrations of VEGF, TGF-β 1 and TGF-β 2 may be closely associated with the efficacy of the strain. VEGF is a heparin-binding glycoprotein involved in vasculogenesis and angiogenesis in the endometrium, which are crucial for embryo implantation and, in fact, impaired endometrial angiogenesis may result in failed implantation and first-trimester abortion during the first three months of pregnancy. TGF-β 1 and TGF-β 2 play well-known roles in the induction of active immune tolerance in mucosal sites. There are high concentrations of both growth factors in human semen, but they require an acidic activation to bind to receptors on vaginal and cervical cells, a fact that is feasible because of the acid pH that characterized the healthy Lactobacillus-dominated vaginal environment. Interestingly, oral administration of L. salivarius MP98 in the trial allowed not only an increase in the vaginal concentrations of TGF-β 1 and TGF-β 2 but, also, a significant acidification of the vaginal pH.

As a conclusion, specific vaginal Lactobacillus strains may display a range of activities with potential to benefit reproductive outcomes, including the competitive exclusion of potentially harmful microbes that may compromise embryo implantation or fertility, contribution to vasculogenesis and angiogenesis, two processes that are required for embryo implantation, and immune-related activities involved in either implantation or tolerance towards the embryo. This highlights the need of a careful strain-by-strain evaluation when probiotics are aimed to contribute to the fertility field, which is very appealing having in account the limited efficacy of the treatments that are available for repetitive abortion and infertility of unknown origin.

### Example 9.2. Administration of Ligilactobacillus salivarius MP101 (=MP98) in an Elderly Nursing Home During the COVID-19 Pandemic: Immunological and Nutritional Impact

Severe Acute Respiratory Syndrome Coronavirus 2, universally known as SARS-CoV-2, was first identified in 2019 as the causative agent of a new acute respiratory disease (coronavirus disease 2019; COVID-19), which was recognized as a pandemic by the World Health Organization on Mar. 11, 2020. It has affected virtually all countries around the world and has caused several millions of deaths. The severity of the disease is higher among people aged ≥60 years, with underlying health conditions, such as hypertension, diabetes, obesity, cardiovascular disease, chronic respiratory disease and weakened immune systems, and/or those living in long-term care facilities. However, most of those infected remain asymptomatic, a fact that complicates the control of the disease.

The fact that SARS-COV-2 initially interacts with mucosal epithelia associated with a complex microbiota (naso-oro-pharyngeal cavities), and that its mechanism of infection is related to the angiotensin-converting enzyme receptor 2 (ACE2), whose activity is influenced and, in turn, influences the microbiota of the upper respiratory and gastrointestinal tracts], suggests an implication of the microbiota in the individual susceptibility to COVID-19 and in the severity of the infection. The modulatory role of the respiratory microbiota has already been observed with other respiratory viruses, including other coronaviruses and the respiratory syncytial virus. In this sense, it has been postulated that people with “eubiotic” respiratory and gastrointestinal microbiotas would be more likely to have an asymptomatic or mild SARS-COV-2 infection given that their microbiotas are associated with appropriate mucosal immune responses. On the contrary, people with respiratory or intestinal dysbiosis would not be able to develop correct responses and would be more prone to a more severe infection and to complications (including cytokine storm, bacterial and fungal secondary infections, and sepsis).

Given that the composition of the microbiota is relevant to our health and that the COVID-19 frame does not seem to be an exception, various articles have postulated the application of strategies stimulating the presence of those members of the respiratory and/or digestive microbiota with beneficial functions for our health and enhancing the mucosal barriers and immune function, such as probiotics. However, most of them simply address/dealt with the benefits of probiotics for general health or speculate about the possible effects that probiotics or some of their metabolites could have on patients who suffer from this infection. In contrast, data from clinical trials specifically designed for the prevention or as an aid in the treatment of COVID-19 through the use of probiotics are very scarce and limited to hospitalized COVID-19 patients.

In any case, COVID-19 contains numerous aspects that constitute clear targets for the application of probiotics including (a) the management of diarrhea and other digestive symptoms that are widespread among patients, (b) the modulation of the immune and inflammatory response to the virus, (c) the prevention of bacterial or fungal co-infections, (d) the prevention or treatment of microbiota alterations associated to the use of respirators, corticosteroids, antibiotics and antifungals, (e) the pre-existing situation of dysbiosis that usually characterizes the population most vulnerable to COVID-19 (elderly, diabetic, immunosuppressed . . . ), and that can also be a consequence of the treatments received, (f) the rise in cases of anxiety, depression or stress associated with the pandemics, and (g) the enhancement of vaccine responses due to adjuvant effects.

In this context, the objective of this work was to investigate the effect of a probiotic strain on the functional, cognitive and nutritional status, and on the nasal and fecal inflammatory profiles of patients living in an elderly nursing home highly affected by COVID-19.

### 9.2.1. Production and Quality Control of the Study Product

In an initial meeting, the director of the elderly nursing home informed that, if possible, the probiotic strain should be administered as a dairy (yogurt-like) product since all residents received a yogurt during dinner. Any other format (capsules, sachets . . . ) would require an individual assessment of product intake, which would imply management problems derived from the reduced available staff when the trial was planned (a high proportion of the staff was in quarantine because of being SARS-COV-2-positive).

As a consequence, a dairy product containing ≥9 log₁₀ colony-forming units (CFU) of L. salivarius MP101 (=MP98) per product unit (125 g) was developed. For this purpose, 50 g of the freeze-dried strain (~9.3 log₁₀ CFU/g) were used to inoculate 100 liters (a batch) of milk previously pasteurized (85° C., 30 minutes), and cooled to 36° C. Yogurt starter cultures were not added. The inoculated milk was homogenized by stirring for 1 h and, then, it was aseptically distributed in 125 g containers, which were fermented at 36° C. for 15 h. The pH values of, at least, two containers were monitored during each batch fermentation. After fermentation, the containers were stored and distributed at refrigeration temperature (2-8° C.). The manufacture of the probiotic dairy product was carried out at the facilities of Villa Villera (Sieso de Huesca, Huesca, Spain), a dairy company that provided altruistically the milk and all the necessary material (containers) and processing (and packaging) equipment.

Four containers from each batch were randomly selected to assess the concentration and purity of the strain at day 1 after production. For this purpose, the content of each container was homogenized and the homogenized product (5 g) was submitted to serial decimal dilutions with sterile peptone water. Aliquots of the dilutions were cultured on plates of de Man Rogosa and Sharpe (MRS) agar (Oxoid, Basingstoke, United Kingdom) for the growth of lactic acid bacteria, polymyxin pyruvate egg-yolk mannitol-bromothymol blue (PEMBA) agar (Oxoid) for the growth of Bacillus spp., MacConkey (MCK) agar (BioMerieux, Marcy l′Etoile, France) for the growth of Gram-negative bacteria, Columbia nalidixic acid (CNA) agar (BioMerieux) for the growth of staphylococci, streptococci, enterococci, corynebacteria and related Gram-positive bacteria, and Sabouraud chloramphenicol dextrose (SDC) agar (BioMerieux) for the growth of yeasts and molds. All the plates were incubated at 37° C. for 48 h. Isolates obtained from MRS plates were identified by 16S rDNA gene amplification and sequencing using a procedure described previously. All the isolates identified as L. salivarius were genotyped by using a Random Amplification of Polymorphic DNA (RAPD) protocol [33]. In addition, the evolution of pH and bacterial counts during cold storage for 42 days was assessed in 4 containers belonging to 6 different batches.

### 9.2.2. Study Design and Participants

The study was carried in an elderly nursing home located in Moralzarzal (Madrid, Spain). This establishment had 47 residents immediately before the pandemic but the population was halved when it reached the location: 18 residents died in a few weeks' period (4 at a hospital and 14 at the nursing home, including 10 with typical COVID-19-associated symptoms) but none of them were PCR tested at that time and, therefore, none of them officially died because of this disease. When the 29 surviving residents were tested for SARS-COV-2-by PCR, most of them were PCR-positive and this was the situation for most of the staff members, too.

In this context, the trial was designed to include all the residents as long as (a) informed consent was obtained from the participants or their legal representatives, (b) they were not fed by parenteral nutrition exclusively and/or (c) they were not allergic to cow's milk proteins (because the probiotic was delivered in a dairy food matrix). A total of 25 residents, aged 74-98, met these criteria and started the trial. Starting at day 0, the residents consumed daily a fermented dairy product (125 g; ~9.3 log₁₀ CFU of L. salivarius MP101 per product) for 4 months. Two samples (nasal wash and feces) were collected from each patient at recruitment (day 0) and at the end of the study (day 120). The nasal wash was obtained using a standardized protocol [34]. Aliquots of the samples were stored at −80° C. until the immunological analyses were performed.

Some demographic and health-related data (sex, age) were recorded at recruitment while others (SARS-COV-2 status, body mass index [BMI], type of diet, concomitant diseases and medication) were recorded both at recruitment and at the end of the study. The staff of the nursing home recorded compliance with the study product intake and the minimum compliance rate (% of days ingesting the study product) was set at 86%. This study was conducted according to the guidelines laid down in the Declaration of Helsinki and was approved by the Ethics Committee of the Hospital Clínico San Carlos (Madrid, Spain) (protocol: CEIC 20/263-E_COVID; date of approval: Jan. 4, 2020, act 4.1/20).

### 9.2.3. Measurement of Functional, Cognitive and Nutritional Status of the Participants

The functional, cognitive and nutritional status of the participants were assessed at recruitment and at the end of the study. The Barthel index (BI) was used to measure the functional status of ten basic activities of daily living (feeding, bathing, dressing, grooming, defecation, urination, toilet use, transfer, mobility, and stairs). The total score ranges from 0 (total dependent living) to 100 (total independent living) [35]. Cognitive, affective and mood status was assessed using the Global Deterioration Scale and Functional Assessment Staging (GDS/FAST) procedure, which includes seven stages, from normal aging (stage 1) to severe dementia (stage 7) [36]. Finally, the nutritional status was evaluated using the Mini Nutritional Assessment (MNA) score [37], which ranges from 0 to 30 points (>24: no nutritional problems; 17-23.5: vulnerable to malnutrition; <17: malnourished).

### 9.2.4. Immunoprofiling of the Nasal and Fecal Samples

Nasal samples (1 mL) were centrifuged and the supernatants were used for the immunological assays. Fecal samples were prepared as described previously [38]. The concentrations of a wide array of inflammation-related immune factors (APRIL/TNFSF13, BAFF/TNFSF13B, Chitinase 3-like 1, IFNα, IFNβ, IFNγ, IL2, IL8, IL10, IL11, IL12p40, IL12p70, IL19, IL20, IL22, IL26, IL27p28, IL28/IFNλ2, IL29/IFNλ1, IL32, IL34, IL35, LIGHT/TNFSF14, MMP-1, MMP-2, MMP-3, osteocalcin, osteopontin, pentraxin 3, TSLP, TWEAK/TNFSF12, gp130/sIL-6Rb, sCD30/TNFRSF8, sCD163, sIL-6Ra, sTNF-R1 and sTNF-R2) were determined using the Bio-Plex Pro Human Inflammation Assay kit (Bio-Rad) in the Bio-Plex 200 instrument (Bio-Rad, Hercules, CA). Every assay was run in duplicate and standard curves were performed for each analyte.

### 9.2.5. Statistical Analysis

Distribution of the data was evaluated using Shapiro-Wilk normality tests. Data with normal distribution were expressed as mean and 95 confidence interval (95% CI), and data with non-normal distribution were expressed as median and interquartile range [IQR]. Microbiological data recorded as CFU per container were log₁₀ transformed before analyses. Kruskal-Wallis tests were used to determine if there were differences in bacterial concentration and pH values among the batches within each sampling time. Friedman's non-parametric repeated measures comparisons followed by post-hoc Nemenyi tests were applied to evaluate differences in these two parameters when different sampling times were compared. In the case of the parameters measured before and after the trial, differences in normally distributed data were assessed using paired t-tests and in non-normally distributed data using Wilcoxon signed-rank tests (library {PairedData version 1.1.1}, respectively. The significance level for the tests was declared at <0.05. Statistical analyses were performed with R-project software, version 4.0.3 (R-project, http://www.r-project.org).

### 9.2.6. Results: Concentration, Purity and Stability of L. salivarius MP101 (=MP98) in the Study Product

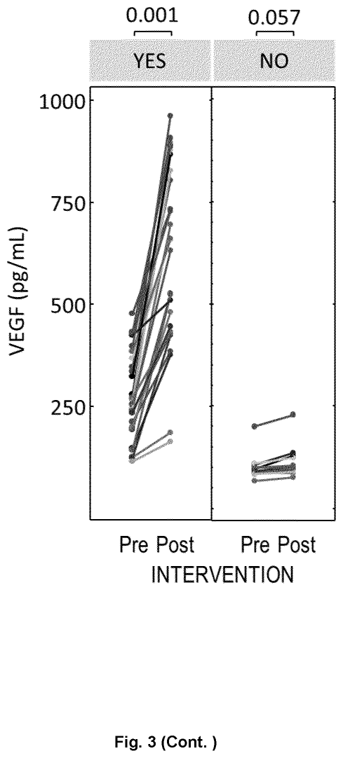

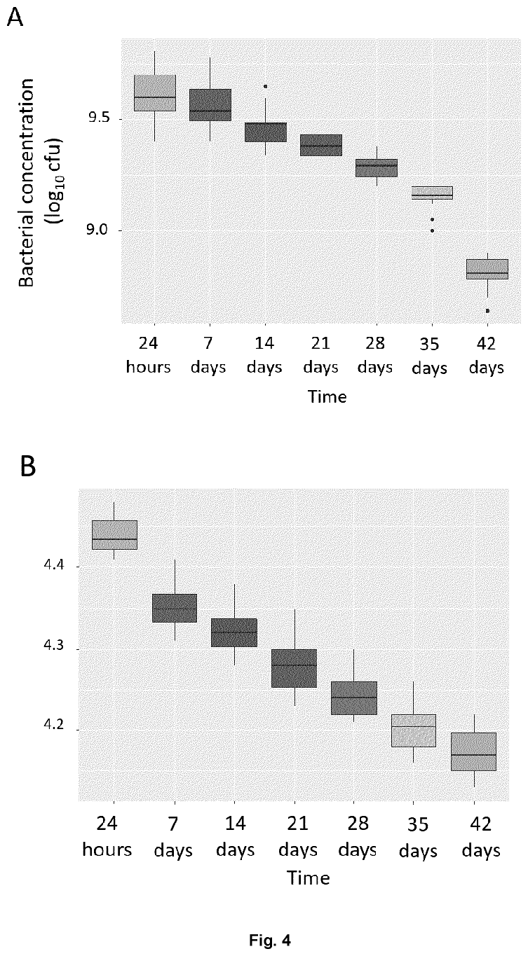

Bacterial concentration (CFU/container) and pH of the dairy products were measured at 24 h and 7, 14, 21, 28, 35 and 42 days after reception of each batch. Microbial growth was only observed on MRS plates while no growth was detected on the remaining media (PEMBA, MCK, CNA, SDC) used to control the purity of the developed product. Median [IQR] values of the MRS counts decreased progressively from 9.60 [9.54-9.70] log₁₀ UFC/mL at 24 h to 8.81 [8.78-8.87] log₁₀ CFU/container at day 42 (FIG. 4A). The median [IQR] of pH value after manufacture (24 h) was 4.43 [4.42-4.46] and dropped to 4.17 [4.17-4.20] after 42 days (FIG. 4B). Both the MRS bacterial counts and the pH values of the probiotic product decreased slowly throughout the cold storage period (FIG. 4) and the differences were statistically significant only when the values obtained at 24 h were compared with those observed after 28, 35 and 42 days (p <0.05 for MRS bacterial counts and pH values, respectively; Friedman's non-parametric repeated measures comparisons followed by post-hoc Nemenyi tests). The MRS counts were above 9 log₁₀ CFU/container (concentration considered as the minimum threshold dose for the trial) until week 5. Consequently, each batch was used for a maximum of 5 weeks after production.

No differences in bacterial counts or in pH were observed between batches within each sampling time (p>0.175 and p>0.353, respectively; Kruskal-Wallis tests), indicating the reproducibility of the manufacturing process.

Only one colony morphology (compatible with that of L. salivarius MP101/MP98) was observed on MRS plates. The identification of the isolates revealed that all of them belonged to the species L. salivarius (>99% identity in the sequence). RAPD genotyping of the L. salivarius isolates revealed that all of them shared the same RAPD profile as the inoculated strain (L. salivarius MP101).

### 9.2.7. Evolution of the COVID-19, Functional, Cognitive and Nutritional Status of the Participants

A total of 22 out of the 25 recruited participants finished the trial (Table 16). One participant died during the first weeks of the trial but she already was in a terminal stage when the trial started (98-years-old; BI: 0; GDS/FAST: 7; MNA: 12.5; skin cancer, COVID-19-positive), while the other two participants moved from the nursing home to a relative's house.

TABLE 16
Main characteristics of the elderly that completed the trial (n = 22).

| Participant Characteristic | Mean (95% CI) or n (%) |
|---|---|
| Age (years) | 84.95 (81.41-88.49) |
| Gender | |
| Male | 11 (50%) |
| Female | 11 (50%) |
| Type of diet | |
| Normal | 10 (45%) |
| Normal/Diabetes | 7 (32%) |
| Pureed foods | 5 (23%) |
| BMI (kg/m²) | |
| Day 0 | 24.82 (22.88-26.751) |
| Day 120 | 24.71 (23.27-26.16) |
| SARS-CoV-2 (positive PCR/total) | |
| Day 0 | 18/22 (81%) |
| Day 120 | 0/22 (0%) |

BMI, body mass index; SARS-CoV-2, Severe Acute Respiratory Syndrome Coronavirus 2.

All the recruited elderly suffered from several, often severe, diseases and were polymedicated. In addition, a high percentage of participants (81%) were SARS-COV-2-positive (mild or asymptomatic disease) at the beginning of the trial while all of them were negative at day 120. Interestingly, none of them became infected or re-infected with SARS-COV-2 throughout the trial despite of the fact that the village where the nursing home is located was confined for several weeks during the period of the trial because the rates were higher than 500 cases per 100,000 inhabitants and/or the rapid spread of the British variant (50%). All the participants received the Pfizer-BioNTech COVID-19 vaccine on 19^{th} January 2021 (first dose) and 9^{th} February 2021 (second dose).

In relation to the functional, cognitive and nutritional status of the participants, there was no change in the GDS/FAST scores of each patient before and after the trial (Table 17). In contrast, the mean or median values for the BI index and the MNA score improved significantly from day 0 (36 and 20.70, respectively) to day 120 (42 and 22.63, respectively) (Table 17).

TABLE 17
Assessment of the functional, cognitive and nutritional status
of the participants at the start and at the end of the trial.
Values are expressed as mean (95% CI) or median [IQR].

| Index or score¹ | Day 0 | Day 120 | p-value |
|---|---|---|---|
| BI | 36.00 [22.75-80.50] | 42.00 [25.25-84.25] | 0.022 |
| GDS/FAST | 4.00 [3.25-6.00] | 4.00 [3.25-6.00] | — |
| MNA | 20.70 (18.80-22.60) | 22.63 (21.20-24.06) | 0.001 |

¹BI, Barthel index; GDS/FAST, Global Deterioration Scale and Functional Assessment Staging; MNA, Mini Nutritional Assessment.

### 9.2.8. Evolution of the Immunological Parameters in the Nasal Samples

In relation to the nasal immunological profiles, the frequency of detection was high (>75% of the samples at both sampling times) for the following immune factors: BAFF/TNFSF13B (which was the only one detected in 100% of the nasal samples), APRIL/TNFSF13, chitinase 3-like 1, IL8, IL32, osteopontin, pentraxin 3, TWEAK/TNFSF12, and gp130/sIL-6Rb (Table 18). In contrast, IFNγ, IL2, IL10, IL20, IL27p28, IL28A/IFNγ2 and IL29A/IFNγ1 were not detected in any of the samples. There were statistically significant differences in the prevalence of several immune factors when samples from day 0 and day 120 were compared. The prevalence decreased over time for IL11, IL12 p70, LIGHT/TNFSF14, MMP-1, TSLP, sTNF-R1 and sTNF-R2, while increased in the case of pentraxin 3 (p≤0.03; Fisher exact tests) (Table 18). BAFF/TNFSF13B, APRIL/TNFSF13 and chitinase 3-like 1 were the immune compounds present at the highest concentrations (μg/L range) in comparison with the rest of the detected factors (ng/L range) (Table 18). The concentrations for BAFF/TNFSF13B, APRIL/TNFSF13, 1, IL8, IL32, osteopontin and sTNF-R1 were lower at the end of the trial (day 120) when compared to the starting time (day 0), and the opposite was observed for chitinase 3-like 1, pentraxin 3, IL19 and IL35 (p≤0.04; paired t-tests or Wilcoxon signed-rank tests) (Table 18).

TABLE 18
Frequency of detection (%) and concentration of immune factors in nasal samples from
the recruited participants before (day 0) and at the end (day 120) of the trial.

| | Day 0 | | Day 120 | | | |
|---|---|---|---|---|---|---|
| | Frequency | Median [IQR] | Frequency | Median [IQR] | | |
| | of | or | of | or | p- | p- |
| Immune factor | detection | Mean (95% CI) | detection | Mean (95% CI) | value^{a} | value |
| APRIL/TNFSF13, [symbol] | 22 (100%) | 7.35 [3.52- [symbol] | 21 (96%) | 6.80 [3.00- [symbol] | >0.99 | <0.00 |
| BAFF/TNFSF13B, [symbol] | 22 (100%) | 2.76 (2.35- [symbol] | 22 [symbol] | 0.89 (0.79- [symbol] | >0.99 | <0.00 |
| Chitinase 3-like 1, [symbol] | 17 (77%) | 0.10 [0.68- [symbol] | 17 (77%) | 0.95 [0.78- [symbol] | >0.99 | 0.040 |
| IFNα2, ng/L | 2 (9%) | 20.05 | 2 (9%) | 20.40 [19.92- [symbol] | >0.99 | — |
| IFNβ, ng/L | 4 (18%) | 2.03 [1.90- [symbol] | 1 (5%) | 1.47 | 0.4 | — |
| IL8, ng/L | 18 (82%) | 2.56 [2.42- [symbol] | 17 (77%) | 0.49 [0.27- [symbol] | >0.99 | <0.00 |
| IL11, ng/L | 7 (32%) | 0.66 [0.52- [symbol] | 0 (0%) | 0 | 0.008 | — |
| IL12p40, ng/L | 4 (18%) | 2.32 [2.14- [symbol] | 0 (0%) | 0 | 0. | — |
| IL12p70, ng/L | 6 (27%) | 0.13 [0.12- [symbol] | 0 (0%) | 0 | 0.02 | — |
| IL19, ng/L | 8 (36%) | 1.55 [0.96- [symbol] | 15 (68%) | 4.76 [3.85- [symbol] | 0.068 | 0.008 |
| IL22, ng/L | 2 (9%) | 2.77 | 2 (9%) | 2.76 | >0.99 | — |
| IL26, ng/L | 0 (0%) | 0 | 1 (5%) | 0.46 | >0.99 | — |
| IL32, ng/L | 18 (82%) | 6.37 (5.32- [symbol] | 17 (77%) | 3.10 (2.29- [symbol] | >0.99 | <0.00 [symbol] |
| IL34, ng/L | 6 (27%) | 20.71 [18.72- [symbol] | 4 (18%) | 12.5 [11.31- [symbol] | 0.72 | — |
| IL35, ng/L | 8 (36%) | 0.64 [0.52- [symbol] | 8 (36%) | 1.14 [1.07- [symbol] | >0.99 | 0.008 [symbol] |
| LIGHT/TNFSF14, [symbol] | 13 (59%) | 1.19 (0.93- [symbol] | 5 (23%) | 0.63 (0.00- [symbol] | 0.03 | 0.684 [symbol] |
| MMP-1, ng/L | 6 (27%) | 17.60 [15.00- [symbol] | 0 (0%) | 0 | 0.02 | — |
| MMP-2, ng/L | 6 (27%) | 34.69 [24.06- [symbol] | 2 (9%) | 14.54 | 0.24 | — |
| MMP-3, ng/L | 4 (18%) | 31.65 [30.05- [symbol] | 0 (0%) | 0 | 0.1 | — |
| Osteocalcin, ng/L | 3 (14%) | 2.24 [1.52- [symbol] | 1 (5%) | 2.97 | 0.61 | — |
| Osteopontin, ng/L | 22 (100%) | 81.22 (69.51- [symbol] | 18 (82%) | 61.74 (49.02- [symbol] | 0.1 | <0.00 [symbol] |
| Pentraxin 3, ng/L | 7 (77%) | 0.71 [0.63- [symbol] | 22 [symbol] | 2.75 [2.28- [symbol] | <0.001 | 0.016 [symbol] |
| TSLP, ng/L | 17 (77%) | 0.73 (0.50- [symbol] | 4 (18%) | 0.42 (0.00- [symbol] | <0.001 | 0.075 [symbol] |
| TWEAK/TNFSF1 [symbol] | 17 (77%) | 2.15 [0.72- [symbol] | 19 (86%) | 2.28 [0.80- [symbol] | 0.69 | 0.579 [symbol] |
| gp130/sIL-6Rb, [symbol] | 22 (100%) | 155.88 [symbol] | 20 (91%) | 157.79 | 0.48 | 0.154 [symbol] |
| sCD30/TNFRSF8, [symbol] | 4 (18%) | 40.84 [14.45- [symbol] | 4 (18%) | 44.02 [19.45- [symbol] | >0.99 | — |
| sCD163, ng/L | 0 (0%) | 0 | 4 (18%) | 2.88 [2.22- [symbol] | 0.1 | — |
| sIL-6Ra, ng/L | 4 (18%) | 10.75 (0-26.46) | 8 (36%) | 11.47 (1.17- [symbol] | 0.31 | 0.351 [symbol] |
| sTNF-R1, ng/L | 21 (96%) | 34.64 (22.76- [symbol] | 13 (59%) | 13.79 (2.58- [symbol] | 0.009 | <0.00 [symbol] |
| sTNF-R2, ng/L | 21 (96%) | 12.82 (9.43- [symbol] | 6(27%) | 4.50 (1.62- [symbol] | <0.001 | 0.212 [symbol] |

^{a}Fisher exact tests were used to evaluate differences in prevalence/detection rates of the analyzed parameters.
ⁿWilcoxon signed-rank tests were used to evaluate differences in concentration of the analyzed parameters with a non-normal distribution.
^{s}Paired t-tests were used to evaluate differences in concentration of the analyzed parameters with a normal distribution.
Bold font indicates values having statistically significant differences. [symbol]
indicates data missing or illegible when filed

### 9.2.9. Evolution of the Immunological Parameters in the Fecal Samples

In relation to the fecal immunological profiles, the frequency of detection was high (>75% of the samples at both sampling times) in a narrower spectrum of immune factors: BAFF/TNFSF13B (the only one detected in 100% of the fecal samples), chitinase 3-like 1, IL32, osteopontin, and gp130/sIL-6Rb (Table 19). Only IL2, IL10, and IL29A/IFNγ1 were not detected in any of the fecal samples. The prevalence decreased from day 0 and day 120 for sTNF-R1 and sTNF-R2, while increased for IFNα2 (p≤0.048; Fisher exact tests) (Table 19).

Similarly to the nasal samples, the immunological compounds BAFF/TNFSF13B, APRIL/TNFSF13 and chitinase 3-like 1 had the highest concentrations (μg/L range) in fecal samples (Table 19). The concentrations of BAFF/TNFSF13B, APRIL/TNFSF13, chitinase 3-like 1, IL32, IL34, gp130/sIL-6Rb, sTNF-R1 and sTNF-R2 decreased from day 0 to day 120 time, while those of pentraxin 3, MMP2, IL19, IL35, and sCD163 increased (p≤0.040; paired t-tests or Wilcoxon signed-rank tests) (Table 19).

TABLE 19
Frequency of detection (%) and concentration of immune factors in fecal samples from the
recruited participants before (day 0) and at the end (day 120) of the trial cited.

| | Day 0 | | Day 120 | | | |
|---|---|---|---|---|---|---|
| | | Median [IQR] | | Median [IQR] | | |
| | Frequency | or | Frequency | or | | |
| | of | Mean | of | Mean | p- | value |
| Immune factor | detection | (95% CI) | detection | (95% CI) | value^{a} | p- |
| APRIL/TNFSF13, [symbol] | 12 (55%) | 1.72 (1.22- [symbol] | 11 (50%) | 1.48 (0.99- [symbol] | >0.99 | 0.0111^{s} |
| BAFF/TNFSF13B, [symbol] | 22 (100%) | 1.21 [0.96- [symbol] | 22 (100%) | 0.49 [0.31- [symbol] | >0.99 | <0.001 |
| Chitinase 3-like 1, [symbol] | 21 (95%) | 0.35 [0.21- [symbol] | 22 (100%) | 0.31 [0.20- [symbol] | >0.99 | 0.0822 |
| IFNα2, ng/L | 0 (0%) | 0 | 5 (23%) | 15.56 [12.12- [symbol] | 0.048 | — |
| IFNβ, ng/L | 6 (27%) | 2.72 ( 1.52- [symbol] | 9 (41%) | 2.82 (1.81- [symbol] | 0.52 | 0.407^{s} |
| IFNγ, ng/L | 4 (18%) | 5.42 [4.09- [symbol] | 0 (0%) | 0 | 0.100 | — |
| IL8, ng/L | 8 (36%) | 3.79 [2.20- [symbol] | 7 (32%) | 1.15 [0.51- [symbol] | >0.99 | — |
| IL11, ng/L | 5 (23%) | 0.36 (0.24- [symbol] | 5 (23%) | 0.36 (0.23- [symbol] | >0.99 | 0.825^{s} |
| IL12p40, ng/L | 8 (36%) | 4.99 (1.89- [symbol] | 9 (41%) | 4.29 (1.59- [symbol] | >0.99 | 0.845^{s} |
| IL12p70, ng/L | 11 (50%) | 0.21 (0.11- [symbol] | 10 (45%) | 0.22 (0.09- [symbol] | >0.99 | 0.823^{s} |
| IL19, ng/L | 7 (32%) | 1.60 [1.19- [symbol] | 14 (64%) | 4.05 [3.54- [symbol] | 0.21 | 0.016ⁿ |
| IL20, ng/L | 1 (5%) | 0.32 | 0 (0%) | 0 | >0.99 | — |
| IL22, ng/L | 8 (36%) | 2.37 (1.21- [symbol] | 10 (45%) | 1.39 (0.66- [symbol] | 0.75 | 0.079^{s} |
| IL26, ng/L | 11 (50%) | 1.28 (0.41- [symbol] | 12 (55%) | 0.91 (0.41- [symbol] | >0.99 | 0.675^{s} |
| IL27p28, ng/L | 9 (41%) | 0.55 [0.44- [symbol] | 8 (36%) | 0.37 [0.21- [symbol] | >0.99 | 0.813ⁿ |
| IL28A/IFNγ2, ng/L | 3 (14%) | 4.52 [4.225- [symbol] | 3 (14%) | 0.73 [0.565- [symbol] | >0.99 | — |
| IL32, ng/L | 21 (95%) | 6.50 [5.89- [symbol] | 20 (91%) | 3.47 [2.67- [symbol] | >0.99 | <0.001 |
| IL34, ng/L | 16 (73%) | 40.57 (30.55- [symbol] | 16 (73%) | 19.19 (11.03- [symbol] | >0.99 | 0.002^{s} |
| IL35, ng/L | 16 (73%) | 7.79 [4.23- [symbol] | 16 (73%) | 10.33 [6.39- [symbol] | >0.99 | 0.005ⁿ |
| LIGHT/TNFSF14, [symbol] | 12 (55%) | 1.39 (0.81- [symbol] | 14 (64%) | 0.87 (0.43- [symbol] | 0.750 | 0.228^{s} |
| MMP-1, ng/L | 7 (32%) | 26.85 [22.37- [symbol] | 3 (14%) | 6.30 [3.96- [symbol] | 0.280 | — |
| MMP-2, ng/L | 10 (45%) | 50.10 (23.35- [symbol] | 6 (27%) | 70.08 (44.45- [symbol] | 0.340 | 0.016^{s} |
| MMP-3, ng/L | 4 (18%) | 36.61 [29.21- [symbol] | 1 (5%) | 23.39 | 0.340 | — |
| Osteocalcin, ng/L | 2 (9%) | 3.27 | 4 (18%) | 2.58 [1.99- [symbol] | 0.660 | — |
| Osteopontin, ng/L | 22 (100%) | 88.61 [75.96- [symbol] | 21 (95%) | 80.70 [74.87- [symbol] | >0.99 | 0.128ⁿ |
| Pentraxin 3, ng/L | 12 (55%) | 0.97 (0.39- [symbol] | 22 (100%) | 2.84 (2.38- [symbol] | 0.34 | <0.001 |
| TSLP, ng/L | 19 (86%) | 0.94 [0.73- [symbol] | 16 (73%) | 0.86 [0.60- [symbol] | 0.450 | 0.551ⁿ |
| TWEAK/TNFSF12, [symbol] | 21 (95%) | 3.19 [2.32- [symbol] | 21 (95%) | 3.69 [2.62- [symbol] | >0.99 | 0.063ⁿ |
| gp130/sIL-6Rb, [symbol] | 20 (91%) | 79.57 (67.48 [symbol] | 20 (91%) | 71.80 (60.79- [symbol] | >0.99 | <0.001 |
| sCD30/TNFRSF8, [symbol] | 11 (50%) | 22.41 [13.51- [symbol] | 13 (59%) | 14.87 [4.45- [symbol] | 0.76 | 0.831ⁿ |
| sCD163, ng/L | 4 (18%) | 5.07 (3.72- [symbol] | 5 (23%) | 6.57(5.72- [symbol] | >0.99 | 0.037^{s} |
| sIL-6Ra, ng/L | 1 (5%) | 10.03 | 6 (27%) | 2.69 [2.35- [symbol] | 0.090 | — |
| sTNF-R1, ng/L | 22 (100%) | 29.62 [19.33- [symbol] | 16 (73%) | 4.98 [1.13- [symbol] | 0.020 | <0.001 |
| sTNF-R2, ng/L | 22 (100%) | 19.72 (15.22- [symbol] | 14 (64%) | 3.25 (1.18- [symbol] | 0.003 | <0.001 |

^{a}Fisher exact tests were used to evaluate differences in prevalence/detection rates of the analyzed parameters.
ⁿWilcoxon signed-rank tests were used to evaluate differences in concentration of the analyzed parameters with a non-normal distribution.
^{s}Paired t-tests were used to evaluate differences in concentration of the analyzed parameters with a normal distribution.
Bold font indicates values having statistically significant differences. [symbol]
indicates data missing or illegible when filed

### 9.2.10. Discussion

Although COVID-19 is a viral infection, emerging evidence indicates that the composition of the respiratory and gastrointestinal microbiotas may be associated to a higher or lower severity of the disease through their immunomodulation and barrier functions. As a consequence, the use of probiotics enhancing such functions has been postulated in the frame of the current pandemic. So far, two studies have found a positive effect of a probiotic product by shortening the duration of diarrheal episodes and decreasing the risk of respiratory failure or by decreasing the risk of death. In this study, a L. salivarius strain specifically selected for the prevention of a viral respiratory infection (RSV-associated bronchiolitis) was tested for the potential functional, nutritional and immunological benefits that might provide to a highly vulnerable elderly population living in a nursing home with a high rate of SARS-COV-2 positive individuals.

In order to minimize the management efforts in the nursing home, the strain was administered as a dairy product, which involved a previous development step. Microbiological analyses of six batches showed that the fermented product only contained L. salivarius MP101 and contained the appropriate amount of viable probiotic cells considered valid for the assay (>9 log₁₀ CFU/container) for five weeks, provided that the probiotic dairy product was stored at refrigeration temperatures. L. salivarius is a species that contains strains with a high probiotic potential as assessed by in vitro and in vivo assays and clinical trials. However, very few strains are commercially available because of their short stability at room temperature. Since the shelf life of fermented milks is usually 4 weeks under refrigeration conditions, the development of a L. salivarius-containing fermented milk may provide a suitable format to provide these benefits to consumers, especially for collectivities in which physical or mental disabilities are widespread, such as elderly nursing homes.

In this study, the daily administration of the probiotic product during four months coincided with a period in which all SARS-COV-2-positives participants became negative and none of them was (re) infected. This fact must be highlighted taking into account that the home was located in a village that was confined for some weeks because of the high rate of COVID-19 infections and that the residents were not vaccinated against SARS-CoV-2 until a few days after the end of the trial.

In addition, probiotic intake led to an improvement of the BI and the MNA score. This is also a relevant finding since studies performed so far have revealed that there is a significant worsening of functional ability in activities of daily living in COVID-19 patients after the infection, regardless of the scale applied, and that older patients are associated to the worse results. Some of these studies were performed using the same tool (BI) employed in our work. Similarly, previous studies have shown that there is a high nutritional risk among COVID-19 patients and that this risk is higher among the elderly. Strengthening of nutritional support during and after COVID-19 seems essential, especially for older adults with diabetes mellitus. Interestingly, the nutritional status (as assessed using the BI) of all the diabetic elderly that participated in our trial improved after probiotic consumption.

In relation to the immunological profiles, the concentrations of a few immune factors changed significantly after the trial. Among them, the decrease in the concentrations of BAFF/TNFSF13B, APRIL/TNFSF13, IL-8, IL31, osteopontin, sTNF-R1 and sTNF-R2, and the increase in those of chitinase 3-like 1, IL19, IL35 and pentraxin 3, seemed particularly relevant since most of them were present in a (relatively) high percentage of both types of samples. The concentration of BAFF/TNFSF13B was significant higher in both nasal and fecal samples before the probiotic intervention. Most participants were SARS-COV-2-positive when recruited and, in addition, some of them suffered from chronic respiratory diseases. BAFF is highly expressed in the airways during infectious and inflammatory processes, including cystic fibrosis and RSV infections, and as a result its concentration increases in samples of bronchoalveolar and nasal lavage fluid obtained from these patients. Increased BAFF levels in fecal samples seem to be a feature of gut inflammation and, in fact, this immune factor may be used as a biomarker for inflammatory bowel diseases. RSV-associated bronchiolitis has also been linked to increased IL8 and sTNF.R1 levels in nasal samples.

The results presented in this work are focused on nasal and fecal samples from elderly living in nursing homes. This is a novelty in the field but at the same time limits the comparison with results obtained by other authors. The nasal environment plays a central role in the acquisition and modulation of SARS-COV-2 infection but information on immune responses developed in the upper respiratory tract against this virus is very scarce. It must be taken into account that COVID-19-related immunoprofiling has been mostly limited to blood samples of hospitalized patients during acute phase infections and that blood and mucosal compartments may behave differently. As an example, high blood levels of pentraxin 3, IL19, and chitinase 3-like 1 have been associated with poor outcome in SARS-COV-2 patients. In contrast, the results of this study showed that the nasal and fecal levels of these immune compounds increased after the probiotic trial although the final values were much lower than those related to poor outcomes in blood samples. In the mucosal compartments, their increases may contribute to clearance of or resistance to pathogens, to tissue repair and to a decrease of the inflammatory state that usually characterize the respiratory and gastrointestinal tracts in elderly. Such health-promoting activities at the respiratory and gut environments may explain the improvements in the functional and nutritional scores observed after the probiotic intervention.

Our results suggest that some immune factors might be used as nasal or fecal biomarkers of probiotic efficacy in SARS-COV-2 elderly but this has to be confirmed in future studies. However, despite the heterogeneity of the recruited participants, a general improvement in the functional, nutritional and immunological status was observed after the trial, which is uncommon in elderly nursing homes highly affected by the COVID-19 pandemic. In conclusion, L. salivarius MP101 (=MP98) is a promising strain as an aid for improving or maintaining health in this highly vulnerable population.

### Example 9.3. Administration of Ligilactobacillus salivarius MP98 to Patients with Recurrent Gout and Comparison with Patients Treated with Allopurinol (Control Group)

A total of 30 patients participated in the study. All of them shared hyperuricemia (>7 mg/dL), a history of recurrent gout episodes (≥3 episodes per year), characterized by acute arthritis and requiring treatment with colchicine despite of taking allopurinol (100-300 mg/daily) as a preventive measure. Definition of case (gout) was performed following the criteria of the Spanish Society for Rheumatology. Half of the patients (n=15; probiotic group) consumed daily, for one year, a sachet containing ~9 log₁₀ CFU of L. salivarius MP98 (Table 20) while the other half of the patients (n=15; control group) consumed allopurinol (100-300 mg/daily) for the same period of time (Table 21).

TABLE 20
Age, BMI and blood biochemical parameters in the probiotic group (MP98) before starting the study.

| | | | | AOPPs | | SH | | | |
|---|---|---|---|---|---|---|---|---|---|
| | | | | (nmol/ | SH | groups | | | 8- |
| | | | AOPPs | mg | groups | (nmol/mg | TBARM | MDA | isoprostaglandin |
| Patient | Age | BMI | (μmol/L) | protein) | (μmol/L) | protein) | (μmol/L) | (μmol/L) | F_{2α} (pg/mL) |
| 1 | 56 | 36.29 | 65.19 | 0.85 | 444.1 | 6.04 | 29.70 | 2.35 | 209.7 |
| 2 | 49 | 29.78 | 59.23 | 0.77 | 430.2 | 5.34 | 25.29 | 1.89 | 169.8 |
| 3 | 51 | 35.16 | 62.40 | 0.82 | 441.8 | 5.67 | 28.27 | 2.21 | 200.1 |
| 4 | 58 | 29.33 | 58.56 | 0.76 | 429.7 | 5.39 | 24.84 | 1.84 | 164.7 |
| 5 | 54 | 32.46 | 61.82 | 0.81 | 433.2 | 5.84 | 27.94 | 2.07 | 197.2 |
| 6 | 50 | 28.74 | 57.21 | 0.75 | 428.5 | 5.27 | 24.03 | 1.80 | 163.6 |
| 7 | 56 | 31.64 | 60.59 | 0.78 | 437.1 | 5.41 | 26.91 | 1.99 | 182.9 |
| 8 | 52 | 28.52 | 56.74 | 0.72 | 427.6 | 5.17 | 23.69 | 1.75 | 166.0 |
| 9 | 58 | 34.98 | 63.04 | 0.81 | 438.3 | 5.47 | 27.03 | 2.14 | 192.7 |
| 10 | 57 | 31.07 | 61.36 | 0.78 | 432.8 | 5.43 | 26.33 | 1.96 | 201.5 |
| 11 | 50 | 33.87 | 60.58 | 0.80 | 435.2 | 5.58 | 27.47 | 2.11 | 190.3 |
| 12 | 48 | 28.02 | 56.72 | 0.71 | 424.4 | 5.11 | 22.52 | 1.72 | 160.4 |
| 13 | 59 | 31.49 | 61.03 | 0.79 | 436.4 | 5.52 | 27.14 | 2.02 | 184.0 |
| 14 | 54 | 35.07 | 61.95 | 0.82 | 439.9 | 5.79 | 29.06 | 2.29 | 204.8 |
| 15 | 56 | 29.20 | 58.76 | 0.76 | 435.0 | 5.44 | 26.01 | 1.92 | 170.1 |

| | | | | | Total | | | |
|---|---|---|---|---|---|---|---|---|
| | NOX | Nitrite | Nitrate | Triglycerides | cholesterol | GOT | GPT | Uric acid |
| Patient | (μmol/L) | (μmol/L) | (μmol/L) | (mg/dl) | (mg/dl) | (UI/L) | (UI/L) | (mg/dl) |
| 1 | 29.97 | 2.26 | 30.12 | 197.20 | 217.03 | 47.64 | 69.27 | 11.35 |
| 2 | 21.03 | 1.90 | 18.91 | 160.11 | 189.03 | 29.11 | 29.20 | 8.04 |
| 3 | 27.98 | 2.21 | 27.47 | 169.09 | 200.07 | 43.20 | 48.21 | 11.42 |
| 4 | 19.95 | 1.86 | 18.07 | 159.27 | 199.06 | 27.08 | 25.09 | 7.58 |
| 5 | 25.90 | 2.18 | 25.08 | 165.44 | 181.29 | 40.57 | 40.03 | 9.87 |
| 6 | 19.64 | 1.77 | 17.54 | 158.17 | 174.82 | 22.14 | 27.48 | 7.24 |
| 7 | 23.51 | 2.00 | 21.73 | 162.85 | 185.04 | 29.07 | 40.07 | 7.42 |
| 8 | 18.89 | 1.75 | 16.48 | 157.52 | 172.15 | 19.05 | 22.73 | 7.29 |
| 9 | 27.02 | 2.20 | 26.88 | 167.93 | 199.24 | 27.07 | 51.98 | 10.91 |
| 10 | 22.69 | 1.98 | 20.07 | 162.10 | 173.27 | 33.62 | 33.45 | 7.97 |
| 11 | 24.79 | 2.14 | 23.14 | 165.83 | 182.41 | 37.08 | 45.51 | 10.23 |
| 12 | 18.03 | 1.71 | 15.74 | 146.89 | 175.04 | 19.76 | 19.01 | 7.11 |
| 13 | 24.67 | 2.10 | 22.40 | 163.97 | 192.82 | 35.90 | 38.22 | 9.02 |
| 14 | 28.86 | 2.24 | 29.07 | 169.81 | 212.07 | 41.33 | 59.96 | 10.88 |
| 15 | 21.99 | 1.92 | 18.99 | 160.93 | 189.99 | 30.24 | 31.62 | 9.25 |

TABLE 21
Age, BMI and blood biochemical parameters in the control group (allopurinol) before starting the study.

| | | | | | | SH | | | |
|---|---|---|---|---|---|---|---|---|---|
| | | | | AOPPs | SH | groups | | | 8- |
| | | | AOPPs | (nmol/mg | groups | (nmol/mg | TBARM | MDA | isoprostaglandin |
| Patient | Age | BMI | (μmol/L) | protein) | (μmol/L) | protein) | (μmol/L) | (μmol/L) | F_{2α} (pg/mL) |
| 1 | 58 | 27.94 | 55.91 | 0.73 | 426.6 | 5.14 | 23.16 | 1.74 | 162.2 |
| 2 | 54 | 30.75 | 57.87 | 0.74 | 425.7 | 5.23 | 22.93 | 1.86 | 161.7 |
| 3 | 53 | 35.41 | 65.77 | 0.83 | 442.0 | 5.84 | 31.02 | 2.41 | 209.0 |
| 4 | 47 | 34.03 | 59.02 | 0.75 | 427.9 | 5.37 | 24.76 | 1.78 | 162.6 |
| 5 | 51 | 32.86 | 62.29 | 0.78 | 441.5 | 5.44 | 26.91 | 2.02 | 177.1 |
| 6 | 59 | 33.91 | 64.83 | 0.81 | 432.7 | 5.45 | 28.14 | 1.99 | 195.2 |
| 7 | 56 | 29.84 | 60.36 | 0.77 | 433.9 | 5.39 | 27.31 | 1.92 | 207.3 |
| 8 | 56 | 28.30 | 58.95 | 0.73 | 425.9 | 5.11 | 24.97 | 1.79 | 160.9 |
| 9 | 52 | 35.87 | 65.81 | 0.84 | 448.3 | 6.10 | 27.28 | 2.41 | 202.4 |
| 10 | 49 | 34.72 | 63.52 | 0.82 | 434.9 | 5.54 | 27.99 | 2.17 | 207.5 |
| 11 | 58 | 34.69 | 61.07 | 0.79 | 438.4 | 5.59 | 27.43 | 2.09 | 194.8 |
| 12 | 57 | 29.33 | 59.50 | 0.75 | 432.7 | 5.42 | 23.07 | 1.83 | 166.1 |
| 13 | 51 | 30.03 | 61.92 | 0.80 | 434.9 | 5.48 | 28.11 | 2.19 | 184.9 |
| 14 | 55 | 30.67 | 64.34 | 0.81 | 435.1 | 5.79 | 28.00 | 2.00 | 195.7 |
| 15 | 57 | 31.40 | 59.71 | 0.77 | 432.2 | 5.51 | 25.79 | 1.87 | 174.4 |

| | | | | | Total | | | Uric |
|---|---|---|---|---|---|---|---|---|
| | NOx | Nitrite | Nitrate | Triglycerides | cholesterol | GOT | GPT | acid |
| Patient | (μmol/L) | (μmol/L) | (μmol/L) | (mg/dl) | (mg/dl) | (UI/L) | (UI/L) | (mg/dl) |
| 1 | 19.01 | 1.73 | 16.85 | 152.07 | 170.45 | 20.94 | 19.32 | 7.03 |
| 2 | 18.98 | 1.81 | 16.77 | 151.28 | 179.39 | 24.52 | 25.75 | 7.53 |
| 3 | 25.67 | 2.41 | 31.12 | 172.29 | 208.16 | 38.57 | 47.78 | 9.97 |
| 4 | 21.48 | 1.79 | 19.64 | 155.83 | 195.37 | 36.71 | 29.19 | 8.44 |
| 5 | 21.66 | 2.12 | 22.48 | 166.74 | 188.31 | 35.83 | 42.81 | 7.56 |
| 6 | 25.18 | 2.11 | 23.99 | 162.41 | 192.43 | 25.11 | 55.07 | 9.99 |
| 7 | 24.07 | 2.06 | 20.29 | 168.02 | 177.63 | 35.37 | 30.64 | 8.44 |
| 8 | 20.01 | 1.71 | 16.93 | 162.33 | 169.04 | 21.62 | 25.55 | 7.81 |
| 9 | 28.23 | 2.14 | 27.46 | 187.99 | 229.46 | 42.75 | 54.98 | 10.64 |
| 10 | 27.09 | 2.27 | 28.65 | 172.25 | 197.18 | 40.28 | 49.91 | 11.69 |
| 11 | 25.56 | 2.14 | 20.92 | 160.86 | 195.37 | 37.01 | 37.56 | 9.20 |
| 12 | 22.29 | 1.84 | 17.03 | 162.25 | 184.41 | 30.17 | 30.07 | 8.24 |
| 13 | 26.86 | 2.21 | 22.52 | 167.27 | 176.25 | 34.88 | 40.39 | 10.05 |
| 14 | 24.48 | 2.11 | 24.81 | 162.91 | 184.30 | 39.64 | 45.29 | 9.92 |
| 15 | 23.16 | 1.92 | 20.02 | 168.28 | 185.19 | 30.53 | 30.37 | 9.06 |

Consumption of drugs used for prevention (allopurinol) or treatment/alliviation (cochicine, NAIDs) of gout was recorded through the study (Tables 22 and 23). Blood samples were obtained at the beginning (T1) and at the end (T2; 12 months) of the study. Blood samples were extracted at Unilabs (Madrid, Spain). The first 8 mL-fraction was collected into a Na-heparin tube to analyze oxidative stress (OS)-related parameters in plasma; a second 4 mL-fraction was used to obtain serum for standard biochemistry. Hematology and biochemical analyses of blood samples were performed by Unilabs. Metabolites related to OS and nitric oxide metabolism end products (NOx) (listed in Table 20 to 23) were measured in duplicate as described previously [39, 40]. The statistic analysis was performed by comparison of repeated measures variances (Friedman's test) using statgraphics.

The results showed that there were no differences in any parameter between T1 and T2 samples for patients belonging to the control group (Table 23). In contrast, there were significant reductions in the levels of several parameters (AOPPs, AOPPs, MDA, 8-isoprostaglandin, triglycerides, total cholesterol, got and uric acid), in the number of gout episodes, and in the consumption of gout-related drugs among patients that received the L. salivarius MP98 strain (Tables 23 and 24; and Table 25).

TABLE 22
Age, recording of gout episodes and drugs intake, and blood biochemical
parameters in the probiotic group (MP98) after finishing the study.

| | | Gout | Gout | | | Doses | | | | | |
|---|---|---|---|---|---|---|---|---|---|---|---|
| | | episodes | episodes | | | ibuprofen | | | | SH | |
| | | (9 | (during | | | and | | AOPPs | SH | groups | |
| | | previous | study | Doses | Doses | similar | AOPPs | (nmol/mg | groups | (nmol/mg | TBARM |
| Patient | Age | months) | period) | Allopurinol | Colchicin | AINS | (μmol/L) | protein) | (μmol/L) | protein) | (μmol/L) |
| 1 | 56 | 3 | 1 | 7 | 8 | 6 | 57.78 | 0.76 | 447.3 | 6.21 | 28.20 |
| 2 | 49 | 2 | 0 | 0 | 0 | 0 | 48.43 | 0.66 | 423.9 | 5.27 | 25.41 |
| 3 | 51 | 1 | 1 | 0 | 4 | 5 | 54.51 | 0.73 | 442.7 | 5.42 | 27.98 |
| 4 | 58 | 1 | 0 | 0 | 0 | 0 | 47.99 | 0.67 | 417.9 | 5.45 | 25.11 |
| 5 | 54 | 1 | 0 | 0 | 0 | 0 | 50.94 | 0.70 | 438.1 | 5.79 | 28.15 |
| 6 | 50 | 2 | 0 | 0 | 0 | 0 | 49.82 | 0.67 | 430.8 | 5.29 | 23.88 |
| 7 | 56 | 1 | 0 | 0 | 0 | 0 | 48.60 | 0.68 | 436.0 | 5.45 | 27.21 |
| 8 | 52 | 0 | 0 | 0 | 0 | 0 | 46.85 | 0.62 | 423.5 | 5.26 | 21.42 |
| 9 | 58 | 3 | 1 | 7 | 6 | 7 | 57.04 | 0.70 | 439.6 | 5.27 | 27.43 |
| 10 | 57 | 1 | 0 | 0 | 0 | 0 | 47.69 | 0.67 | 424.7 | 5.38 | 25.76 |
| 11 | 50 | 1 | 0 | 0 | 0 | 0 | 48.71 | 0.71 | 437.1 | 5.51 | 26.57 |
| 12 | 48 | 1 | 0 | 0 | 0 | 0 | 47.99 | 0.62 | 429.7 | 5.23 | 22.93 |
| 13 | 59 | 2 | 1 | 0 | 4 | 5 | 51.14 | 0.67 | 431.8 | 5.57 | 27.00 |
| 14 | 54 | 2 | 1 | 10 | 8 | 10 | 57.01 | 0.72 | 432.6 | 5.60 | 29.07 |
| 15 | 56 | 2 | 0 | 0 | 0 | 0 | 48.09 | 0.68 | 437.3 | 5.41 | 24.82 |

| | | 8- | | | | | Total | | | Uric |
|---|---|---|---|---|---|---|---|---|---|---|
| | MDA | isoprostaglandin | NOX | Nitrite | Nitrate | Triglycerides | cholesterol | GOT | GPT | acid |
| Patient | (μmol/L) | F_{2α} (pg/ml) | (μmol/L) | (μmol/L) | (μmol/L) | (mg/dl) | (mg/dl) | (UI/L) | (UI/L) | (mg/dl) |
| 1 | 2.09 | 180.1 | 29.07 | 2.29 | 30.87 | 149.09 | 187.46 | 45.35 | 62.83 | 8.42 |
| 2 | 1.82 | 151.7 | 21.24 | 2.00 | 18.07 | 139.29 | 154.13 | 27.19 | 30.27 | 7.49 |
| 3 | 2.08 | 178.9 | 26.07 | 2.14 | 26.21 | 127.91 | 182.12 | 41.03 | 46.12 | 8.95 |
| 4 | 1.79 | 145.6 | 21.06 | 1.91 | 19.14 | 134.32 | 164.27 | 28.14 | 26.91 | 7.04 |
| 5 | 1.97 | 162.5 | 23.89 | 2.07 | 24.85 | 140.01 | 159.10 | 28.86 | 37.94 | 8.69 |
| 6 | 1.85 | 145.7 | 20.73 | 1.72 | 17.45 | 136.26 | 150.92 | 21.84 | 26.39 | 6.74 |
| 7 | 1.83 | 164.7 | 20.91 | 2.13 | 22.93 | 128.74 | 145.04 | 27.97 | 38.96 | 7.07 |
| 8 | 1.71 | 142.1 | 19.07 | 1.71 | 16.82 | 131.36 | 166.01 | 20.56 | 22.88 | 6.94 |
| 9 | 1.96 | 170.4 | 26.23 | 2.12 | 25.95 | 127.69 | 161.35 | 26.12 | 46.07 | 9.02 |
| 10 | 1.82 | 180.6 | 21.78 | 2.07 | 20.74 | 135.54 | 151.36 | 30.37 | 33.71 | 7.01 |
| 11 | 1.90 | 169.2 | 23.80 | 2.01 | 21.21 | 148.47 | 157.52 | 35.83 | 42.06 | 9.00 |
| 12 | 1.67 | 144.6 | 19.00 | 1.79 | 15.98 | 122.04 | 142.29 | 21.45 | 21.11 | 6.72 |
| 13 | 1.93 | 162.1 | 21.78 | 2.04 | 22.11 | 141.80 | 167.07 | 32.78 | 35.53 | 8.29 |
| 14 | 2.05 | 174.3 | 27.65 | 2.29 | 26.69 | 133.83 | 188.58 | 36.97 | 49.69 | 8.94 |
| 15 | 1.84 | 142.9 | 20.12 | 1.86 | 19.29 | 127.94 | 147.84 | 29.64 | 29.46 | 8.14 |

TABLE 23
Age, recording of gout episodes and drugs intake, and blood biochemical parameters in the control group (allopurinol) after finishing the study.

| | | Gout | Gout | | | | | | | | |
|---|---|---|---|---|---|---|---|---|---|---|---|
| | | episodes | episodes | | | Doses | | | | SH | |
| | | (9 | (during | | | ibuprofen | | AOPPs | SH | groups | |
| | | previous | study | Doses | Doses | and similar | AOPPs | (nmol/mg | groups | (nmol/mg | TBARM |
| Patient | Age | months) | period) | Allopurinol | Colchicin | AINS | (μmol/L) | protein) | (μmol/L) | protein) | (μmol/L) |
| 1 | 58 | 1 | 0 | 180 | 0 | 0 | 54.87 | 0.72 | 429.5 | 5.19 | 22.89 |
| 2 | 54 | 2 | 1 | 175 | 4 | 8 | 57.96 | 0.75 | 423.6 | 5.21 | 23.48 |
| 3 | 53 | 2 | 2 | 174 | 8 | 12 | 66.12 | 0.83 | 448.1 | 5.72 | 29.92 |
| 4 | 47 | 1 | 0 | 168 | 0 | 0 | 58.27 | 0.76 | 429.7 | 5.41 | 27.01 |
| 5 | 51 | 0 | 1 | 178 | 4 | 6 | 62.35 | 0.78 | 435.4 | 5.42 | 27.83 |
| 6 | 59 | 2 | 2 | 180 | 8 | 16 | 64.76 | 0.80 | 430.2 | 5.41 | 27.59 |
| 7 | 56 | 2 | 1 | 179 | 4 | 6 | 60.56 | 0.77 | 431.0 | 5.36 | 29.04 |
| 8 | 56 | 1 | 1 | 172 | 2 | 4 | 57.71 | 0.75 | 426.3 | 5.23 | 25.67 |
| 9 | 52 | 2 | 3 | 174 | 12 | 1 | 65.70 | 0.83 | 451.8 | 6.02 | 25.91 |
| 10 | 49 | 3 | 2 | 175 | 8 | 6 | 63.68 | 0.82 | 436.7 | 5.59 | 28.75 |
| 11 | 58 | 2 | 1 | 176 | 4 | 3 | 60.94 | 0.80 | 436.1 | 5.48 | 26.32 |
| 12 | 57 | 1 | 1 | 179 | 4 | 6 | 59.37 | 0.76 | 433.4 | 5.47 | 24.81 |
| 13 | 51 | 2 | 1 | 177 | 4 | 3 | 61.97 | 0.81 | 436.8 | 5.43 | 27.09 |
| 14 | 55 | 2 | 2 | 180 | 6 | 12 | 64.14 | 0.81 | 432.7 | 5.74 | 28.27 |
| 15 | 57 | 1 | 1 | 180 | 2 | 4 | 59.82 | 0.78 | 431.3 | 5.52 | 24.86 |

| | | 8- | | | | | Total | | | Uric |
|---|---|---|---|---|---|---|---|---|---|---|
| | MDA | isoprostaglandin | NOx | Nitrite | Nitrate | Triglycerides | cholesterol | GOT | GPT | acid |
| Patient | (μmol/L) | Fzs (pg/ml) | (μmol/L) | (μmol/L) | (μmol/L) | (mg/dl) | (mg/dl) | (UI/L) | (UI/L) | (mg/dl) |
| 1 | 1.78 | 166.1 | 21.32 | 1.82 | 16.92 | 157.81 | 172.63 | 20.05 | 19.97 | 7.11 |
| 2 | 1.83 | 162.9 | 19.26 | 1.87 | 16.86 | 153.17 | 176.04 | 22.63 | 27.46 | 7.46 |
| 3 | 2.37 | 205.9 | 23.87 | 2.29 | 28.79 | 169.90 | 206.72 | 37.84 | 44.90 | 9.68 |
| 4 | 1.84 | 164.8 | 22.75 | 1.67 | 19.85 | 158.47 | 196.82 | 37.28 | 27.61 | 8.87 |
| 5 | 2.14 | 175.0 | 21.13 | 2.03 | 21.04 | 165.58 | 183.24 | 33.57 | 43.27 | 7.64 |
| 6 | 2.09 | 192.4 | 24.97 | 2.24 | 23.87 | 164.25 | 194.32 | 27.60 | 53.45 | 9.61 |
| 7 | 1.84 | 204.7 | 24.59 | 2.27 | 22.81 | 162.81 | 176.47 | 37.24 | 34.73 | 8.69 |
| 8 | 1.81 | 165.2 | 22.33 | 1.84 | 17.40 | 165.45 | 170.51 | 21.94 | 27.08 | 7.90 |
| 9 | 2.29 | 205.1 | 26.43 | 2.02 | 26.73 | 189.72 | 224.95 | 40.68 | 59.99 | 10.92 |
| 10 | 2.22 | 207.1 | 28.07 | 2.22 | 27.47 | 171.63 | 199.08 | 41.07 | 53.87 | 10.87 |
| 11 | 2.01 | 195.9 | 24.07 | 2.19 | 22.10 | 154.57 | 193.84 | 39.21 | 35.57 | 9.12 |
| 12 | 1.74 | 164.5 | 24.81 | 1.77 | 17.49 | 165.16 | 181.94 | 31.82 | 27.61 | 8.57 |
| 13 | 2.27 | 186.3 | 25.79 | 2.26 | 21.36 | 163.83 | 178.63 | 32.99 | 39.40 | 10.49 |
| 14 | 2.08 | 191.9 | 25.95 | 2.20 | 25.27 | 160.02 | 187.14 | 38.57 | 44.47 | 9.38 |
| 15 | 1.81 | 175.8 | 22.68 | 1.88 | 20.31 | 168.91 | 183.02 | 32.42 | 33.84 | 9.12 |

TABLE 24
Episodes of gout during the 9 months previous to
the study and during the 9 months of the study.

| | Previous | Study | P-value* (study 9 |
|---|---|---|---|
| | 9 months | 9 months | months) |
| Control group | 24 | 19 | 0.03857 |
| Probiotic group | 23 | 5 | |

*Two-tailed Fisher exact test.

TABLE 25
Concentration of several oxidative stress (OS)-related parameters in
plasma samples obtained from the patients recruited in this study.

| | Control | Probiotic | Control | Probiotic | P- | P- |
|---|---|---|---|---|---|---|
| | group | group | group | group | value | value |
| | (initial) | (initial) | (final) | (final) | 1^{a} | 2^{a} |
| AOPPs (μmol/L) | 61.07 | 60.59 | 60.94 | 48.71 | 0.796 | <0.001 |
| | [59.26-63.93] | [58.66-61.89] | [58.82-63.91] | [48.04-52.83] | | |
| AOPPs | 0.78 | 0.78 | 0.78 | 0.68 | 0.206 | <0.001 |
| (nmol/mg | [0.75-0.81] | [0.76-0.81] | [0.76-0.81] | [0.67-0.71] | | |
| protein) | | | | | | |
| SH groups | 433.90 | 435.00 | 432.70 | 432.60 | 0.796 | 0.796 |
| (μmol/L) | [430.05-436.75] | [429.95-437.70] | [429.95-436.40] | [427.20-437.70] | | |
| SH groups | 5.45 | 5.44 | 5.43 | 5.42 | 0.439 | 0.796 |
| (nmol/mg | [5.38-5.57] | [5.37-5.63] | [5.39-5.56] | [5.28-5.54] | | |
| protein) | | | | | | |
| TBARM | 27.28 | 26.91 | 27.01 | 26.57 | 0.796 | 0.796 |
| (μmol/L) | [24.86-28.00] | [25.07-27.71] | [25.27-28.05] | [24.97-27.71] | | |
| MDA (μmol/L) | 1.99 | 1.99 | 2.01 | 1.85 | 0.796 | <0.001 |
| | [1.85-2.13] | [1.87-2.13] | [1.82-2.18] | [1.82-1.97] | | |
| 8-isoprostaglandin | 184.90 | 184.00 | 186.30 | 162.50 | 0.796 | <0.001 |
| F2 (pg/mL) | [164.35-199.05] | [167.90-198.65] | [165.65-200.30] | [145.65-172.35] | | |
| NOx (μmol/L) | 24.07 | 23.51 | 24.07 | 21.78 | 0.796 | 0.197 |
| | [21.57-25.615] | [20.49-26.46] | [22.50-25.38] | [20.82-24.98] | | |
| Nitrite (μmol/L) | 2.11 | 2.00 | 2.03 | 2.04 | 0.796 | 0.796 |
| | [1.825-2.14] | [1.88-2.19] | [1.86-2.23] | [1.89-2.13] | | |
| Nitrate (μmol/L) | 20.92 | 21.73 | 21.36 | 21.21 | 0.439 | 0.796 |
| | [18.33-24.4] | [18.49-25.98] | [18.67-24.57] | [18.61-25.40] | | |
| Triglycerides | 162.91 | 162.85 | 164.25 | 134.32 | 0.796 | <0.001 |
| (mg/dL) | [161.55-168.15] | [159.69-166.88] | [159.25-167.25] | [128.34-139.65] | | |
| Total colesterol | 185.19 | 189.03 | 183.24 | 159.10 | 0.796 | <0.001 |
| (mg/dL) | [178.51-195.37] | [178.165-199.15] | [177.55-195.57] | [151.14-166.54] | | |
| GOT (UI/L) | 35.37 | 30.24 | 33.57 | 28.86 | 0.796 | 0.02 |
| | [27.64-37.79] | [27.08-38.83] | [29.71-38.21] | [26.66-34.31] | | |
| GPT (UI/L) | 37.56 | 38.22 | 35.57 | 35.53 | 0.796 | 0.197 |
| | [29.63-46.54] | [28.34-46.86] | [27.61-44.69] | [28.19-44.07] | | |
| Uric acid | 9.06 | 9.02 | 9.12 | 8.14 | 0.438 | <0.001 |
| (mg/dL) | [8.025-9.98] | [7.50-10.56] | [8.24-9.65] | [7.03-8.82] | | |

^{a}P-value 1: Comparison between Control (initial) and Control (final); P-value 2: Comparison between Probiotic (initial) and Probiotic (final). Analysis performed by using the Friedman's test and Statgraphics. There were no statistically significant differences between the control group (initial) and the probiotic group (initial) for any of the tested parameters,

### Example 10. Comparison of the Vaginal Effects of L. salivarius CECT5713 and MP98

Comparison on some vaginal parameters on women who undertook the clinical trials with L. salivarius MP98 and L. salivarius CECT5713 is shown in table 26. Parameters as the Nugent score, TGF-β1, TGF-β2, VEGF, viable counts in L. salivarius MP98 are closer to the control cases than those with L. salivarius CECT5713. Afterall, the pregnancy effectiveness and the reproductive success are higher when L. salivarius MP98 is used (67.5% vs 66%, and 62.5% vs 57%)

TABLE 26
Comparison of the vaginal parameters between women who were able to complete a
full-term pregnancy (n = 25) and those who did not (n = 19) after the intervention
with L. salivarius MP98 (n = 40) or L. salivarius CECT 5713 (n = 44).

| | | Probiotic intervention | | Probiotic intervention | |
|---|---|---|---|---|---|
| | | resulted in pregnancy | | resulted in pregnancy (L. | |
| | Control | (L. salivarius MP98) | | salivarius CECT5713)* | |
| | (n = 14) | Yes (n = 25) | No (n = 15) | Yes (n = 25) | No (n = 19) |
| | Mean | Mean | Mean | Mean | Mean |
| | (95% CI) | (95% CI) | (95% CI) | (95% CI) | (95% CI) |
| pH | 4.53 (0.15) | 4.44 (0.12) | 5.80 (0.22) | 4.48 (0.23) | 5.78 (0.34) |
| Nugent score | 1.79 (0.52) | 2.04 (0.49) | 5.87 (0.62) | 2.36 (1.08) | 5.84 (1.01) |
| TGF-β1 (pg/mL) | 4.83 (0.18) | 4.40 (0.21) | 2.21 (0.19) | 4.36 (0.39) | 2.27 (0.43) |
| TGF-β2 (pg/mL) | 3.22 (0.12) | 2.94 (0.17) | 1.33 (0.15) | 2.88 (0.32) | 1.27 (0.26) |
| VEGF (pg/mL) | 406.0 (84) | 662.72 (121.35) | 121.87 (25.85) | 755 (285) | 103 (43) |
| Lactobacilli | 14 (100) | 25 (100) | 6 (40) | 25 (100) | 10 (46) |
| Positive | | | | | |
| women [n (%)] | | | | | |
| Viable counts | 7.24 (0.35) | 7.04 (0.3) | 4.82 (0.77) | 6.47 (0.60) | 4.87 (1.45) |
| (log₁₀ | | | | | |
| CFU/swab) | | | | | |
| L. salivarius | — | 25 (100) | 7 (47) | 25 (100) | 7 (37) |
| qPCR [n (%)] | | | | | |
| L. salivarius | — | 7.51 (0.68) | 3.37 (0.71) | 6.70 (0.56) | 3.16 (0.88) |
| qPCR | | | | | |
| (copies/swab) | | | | | |
| Pregnancy | — | 27/40 | 13/40 | 29/44 | 15/44 |
| (events/total | | (total RA + | | (total RA + | |
| events) | | INF) | | INF) | |
| Pregnancy | — | 67.5% | — | 66% | — |
| effectiveness | | | | | |
| Successful | — | 25/40 | 15/40 | 25/44 | 19/44 |
| pregnancy | | (total RA + | | (total RA + | |
| (events/total | | INF) | | INF) | |
| events) | | | | | |
| Reproductive | — | 62.5% | — | 57% | — |
| success | | | | | |

*Source WO 2021/250289
RA: repetitive abortion
INF: Infertility

### Example 11

### 10.1 Origin and Characterization of Strain ES43

The strain ES43, deposited at the “Colección Española de Cultivos Tipo (CECT)” under the accession number CECT 30641, was isolated from breast milk in a previous project of the research group. Its taxonomic identification was carried out by means of the partial sequencing of the 16S rDNA gene. To that end, DNA was extracted from a colony of a pure culture of the strain using a commercial kit (DNA DNEASY, QIAGEN). A PCR was then performed to amplify the desired fragment and after confirming that the extraction and amplification were performed successfully by means of gel electrophoresis, the PCR product was purified with a commercial kit (QIAPREP, QIAGEN). The resulting DNA was delivered to the company Stab Vidas (Portugal) to obtain the nucleotide sequence of the amplified fragment. The FASTA file provided was entered in the NCBI BLASTn database for aligning the sequences with those existing in the database. This analysis confirmed that the isolated strain belonged to the species Ligilactobacillus salivarius (query cover (% of the query sequence which is in alignment with the identified sequence >99%).

The 16sRNA gene of L. salivarius CECT 30641 has the sequence shown in SEQ ID NO: 3

(SEQ ID NO: 3)
ACCTTATGACAATTCTCATCACTCGTGTTCTTCTCTAACAACAGAGTTTTACGATCC
GAAGACCTTCTTCACTCACGCGGCGTTGCTCCATCAGACTTGCGTCCATTGTGGA
AGATTCCCTACTGCTGCCTCCCGTAGGAGTTTGGGCCGTGTCTCAGTCCCAATGT
GGCCGATCAACCTCTCAGGTCGGCTACGTATCATCACCTTGGTAGGCCGTTACCC
CACCAACTAGTTAATACGCCGCGGGGACCTCTAAAAGCGATAGCAGAAACCTCTT
TCTTCTAAGGAACCCGCGATCCTCAAAAG

A total of 11 strains were selected for determination of metabolic activity of estrogens. The selection derives from a preliminary analysis where the growth kinetics (umax), the generation rate (g) and microbial counts of the maximum cell population reached after 24 h of incubation (24 h, log ufc/ml) of several lactic bacterial strains were determined. Strains with slow and moderate growth were excluded at this stage. Strains were also evaluated for the resistance to the most widely used antibiotics. The enzymatic activities evaluated were the β-glucosidase and β-glucuronidase. Strains with different patterns of enzymatic activities, were selected for determination of their capacity for estrogens metabolic activity. +++means the highest enzymatic activity and/-the lower enzymatic activity (Table 27).

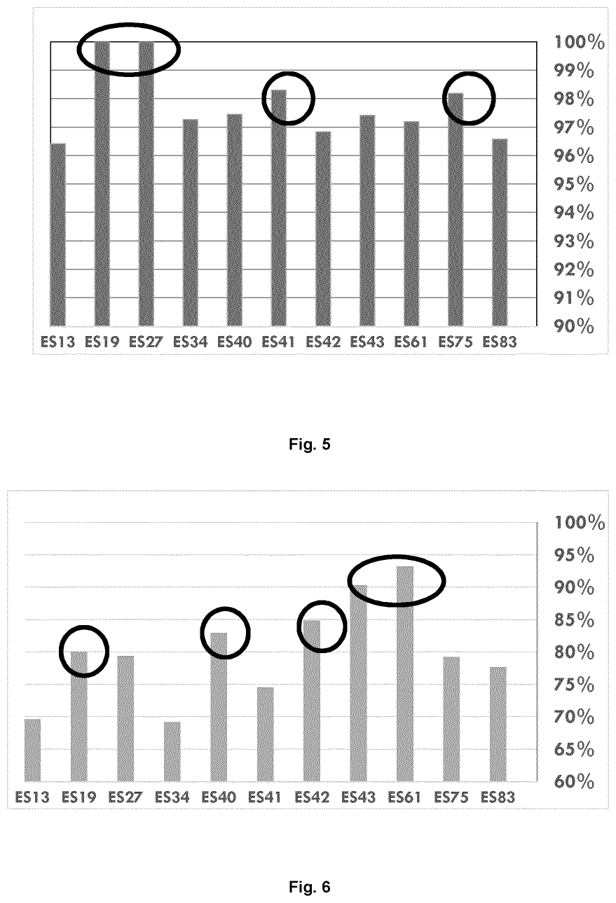

Table 28, Table 29 and FIG. 5 show the estrone determination measured by HPLC/MS or metabolization (M in ppms) of estrone in the different strains selected. M is considered to be the initial estrogens (i)—the free estrogens (L). % or metabolization rate is considered as metabolized estrogens/initial estrogens. ES19, ES27 corresponding to “MP 98” strain), ES 41 and ES75 are highlighted because they are 100% or very close to 100% metabolization. No biotransformation of estrone to the other primary estrogens were detected.

TABLE 28
Estrone (i = 26 ppm)

| | | | C | | M | % M |
|---|---|---|---|---|---|---|
| Strain | Code | L | (T − L) | T | (i − L) | (M/i) |
| Lactobacillus | ES13 | 0.93 | 0.03 | 0.96 | 25.07 | 96% |
| gasseri | | | | | | |
| Lactobacillus | ES19 | 0.00 | 0.94 | 0.94 | 26.00 | 100% |
| johnsonii | | | | | | |
| Ligilactobacillus | ES27 | 0.00 | 0.84 | 0.84 | 26.00 | 100% |
| salivarius | | | | | | |
| Ligilactobacillus | ES34 | 0.71 | 0.65 | 1.36 | 25.29 | 97% |
| salivarius | | | | | | |
| Ligilactobacillus | ES40 | 0.66 | 0.94 | 1.60 | 25.34 | 97% |
| salivarius | | | | | | |
| Ligilactobacillus | ES41 | 0.44 | 0.02 | 0.46 | 25.56 | 98% |
| salivarius | | | | | | |
| Ligilactobacillus | ES42 | 0.82 | 0.13 | 0.95 | 25.18 | 97% |
| salivarius | | | | | | |
| Ligilactobacillus | ES43 | 0.67 | 0.16 | 0.83 | 25.33 | 97% |
| salivarius | | | | | | |
| Limosilactobacillus | ES61 | 0.73 | 0.22 | 0.95 | 25.27 | 97% |
| reuteri | | | | | | |
| Pediococcus | ES75 | 0.47 | 0.22 | 0.69 | 25.53 | 98% |
| acidilactici | | | | | | |
| Lactobacillus | ES83 | 0.89 | 0.23 | 1.12 | 25.11 | 97% |
| crispatus | | | | | | |

Tables 30, 31 and FIG. 6 show the estriol determination measured by HPLC/MS or metabolization (M in ppms) of estriol in the different strains selected. M is considered to be the initial estrogens (i)—the free estrogens (L). % or metabolization rate is considered as metabolized estrogens/initial estrogens. ES19, ES40, ES 42, ES43 and ES61 are highlighted because they shows the highest rate of metabolization of estriol. It was detected biotransformation of estriol to 17β-estradiol for the strain ES75.

TABLE 30
Results estriol (i = 28 ppm)

| | | | C | | M | % M |
|---|---|---|---|---|---|---|
| Strain | Code | L | (T − L) | T | (i − L) | (M/i) |
| Lactobacillus gasseri | ES13 | 8.52 | 3.18 | 11.70 | 19.48 | 70% |
| Lactobacillus | ES19 | 5.60 | 6.39 | 11.99 | 22.40 | 80% |
| johnsonii | | | | | | |
| Ligilactobacillus | ES27 | 5.79 | 5.18 | 10.97 | 22.21 | 79% |
| salivarius | | | | | | |
| Ligilactobacillus | ES34 | 8.64 | 0.18 | 8.82 | 19.36 | 69% |
| salivarius | | | | | | |
| Ligilactobacillus | ES40 | 4.79 | 5.74 | 10.53 | 23.21 | 83% |
| salivarius | | | | | | |
| Ligilactobacillus | ES41 | 7.14 | 4.71 | 11.85 | 20.86 | 75% |
| salivarius | | | | | | |
| Ligilactobacillus | ES42 | 4.25 | 2.03 | 6.28 | 23.75 | 85% |
| salivarius | | | | | | |
| Ligilactobacillus | ES43 | 2.73 | 5.53 | 8.26 | 25.27 | 90% |
| salivarius | | | | | | |
| Limosilactobacillus | ES61 | 1.91 | 1.16 | 3.07 | 26.09 | 93% |
| reuteri | | | | | | |
| Pediococcus | ES75 | 5.83 | 1.85 | 7.68 | 22.17 | 79% |
| acidilactici | | | | | | |
| Lactobacillus | ES83 | 6.27 | 0.76 | 7.03 | 21.73 | 78% |
| crispatus | | | | | | |

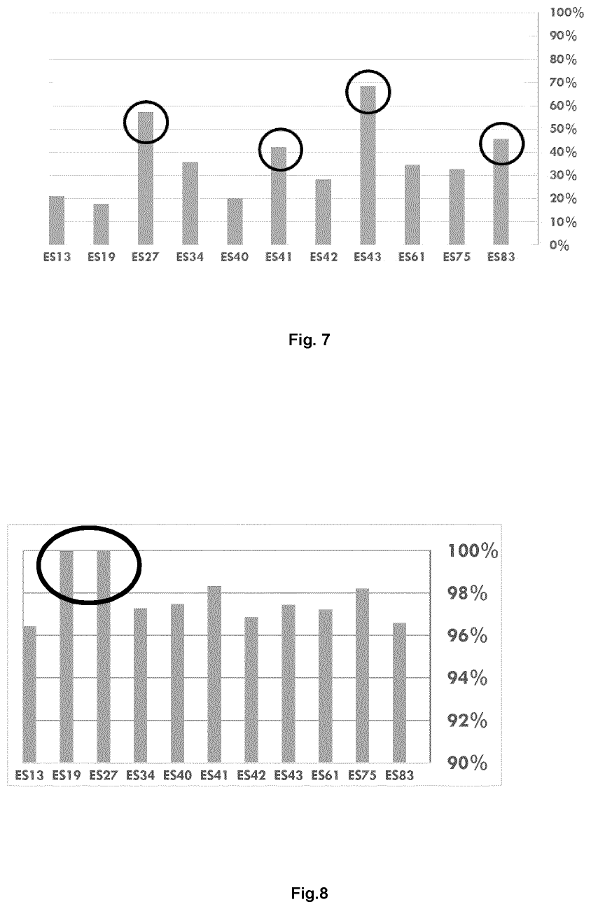

Tables 32, 33 and FIG. 7 show the 17β-estradiol metabolization (M in ppms) measured by HPLC/MS in the different strains selected, M is considered to be the initial estrogens (i)—the free estrogens (L). % or metabolization rate is considered as metabolized estrogens/initial estrogens. ES27 corresponding to “MP 98” strain), ES41, ES43 and ES83 are highlighted because they shows the highest rate of metabolization of estriol. It was detected and quantified biotransformation of 17β-estradiol to estriol by strains ES27 corresponding to “MP 98” strain), 34, 40, 41, 42, 43, 61 and 75.

TABLE 32
Results 17β-estradiol (i = 30 ppm)

| | | | C | | M | % M |
|---|---|---|---|---|---|---|
| Strain | Code | L | (T − L) | T | (i − L) | (M/i) |
| Lactobacillus | ES13 | 23.68 | 30.41 | 54.09 | 6.32 | 21% |
| gasseri | | | | | | |
| Lactobacillus | ES19 | 24.69 | 15.38 | 40.07 | 5.31 | 18% |
| johnsonii | | | | | | |
| Ligilactobacillus | ES27 | 12.90 | 10.28 | 23.18 | 17.10 | 57% |
| salivarius | | | | | | |
| Ligilactobacillus | ES34 | 19.34 | 12.85 | 32.19 | 10.66 | 36% |
| salivarius | | | | | | |
| Ligilactobacillus | ES40 | 23.96 | 11.39 | 35.35 | 6.04 | 20% |
| salivarius | | | | | | |
| Ligilactobacillus | ES41 | 17.37 | 11.29 | 28.66 | 12.63 | 42% |
| salivarius | | | | | | |
| Ligilactobacillus | ES42 | 21.55 | 10.58 | 32.13 | 8.45 | 28% |
| salivarius | | | | | | |
| Ligilactobacillus | ES43 | 9.54 | 14.50 | 24.04 | 20.46 | 68% |
| salivarius | | | | | | |
| Limosilactobacillus | ES61 | 19.68 | 10.77 | 30.45 | 10.32 | 34% |
| reuteri | | | | | | |
| Pediococcus | ES75 | 20.25 | 15.98 | 36.23 | 9.75 | 33% |
| acidilactici | | | | | | |
| Lactobacillus | ES83 | 16.30 | 11.61 | 27.91 | 13.70 | 46% |
| crispatus | | | | | | |

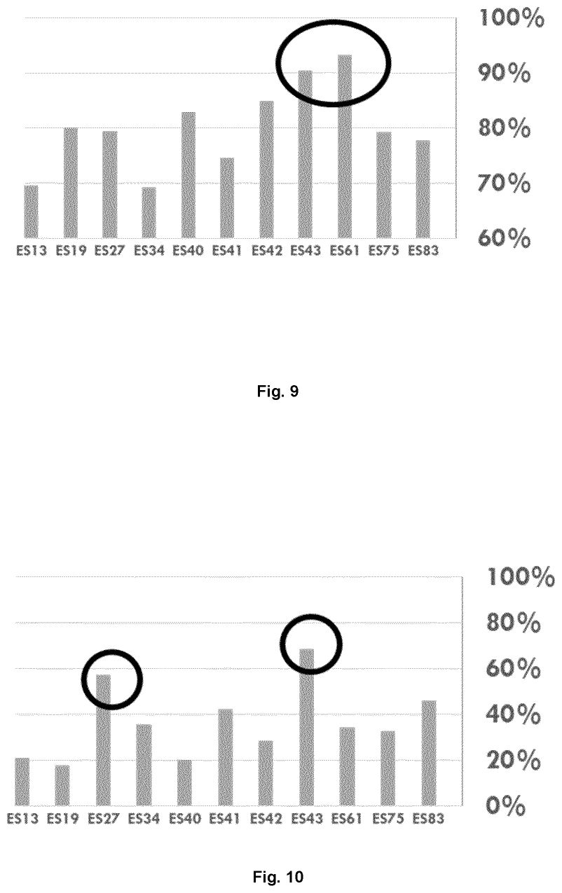

The global assessment of primary estrogen (estrone, estriol and 17β-estradiol) metabolization rate is shown in Tables 34-36 and FIGS. 8-10. Those strains with higher estrogen metabolization rates as a whole are highlighted.

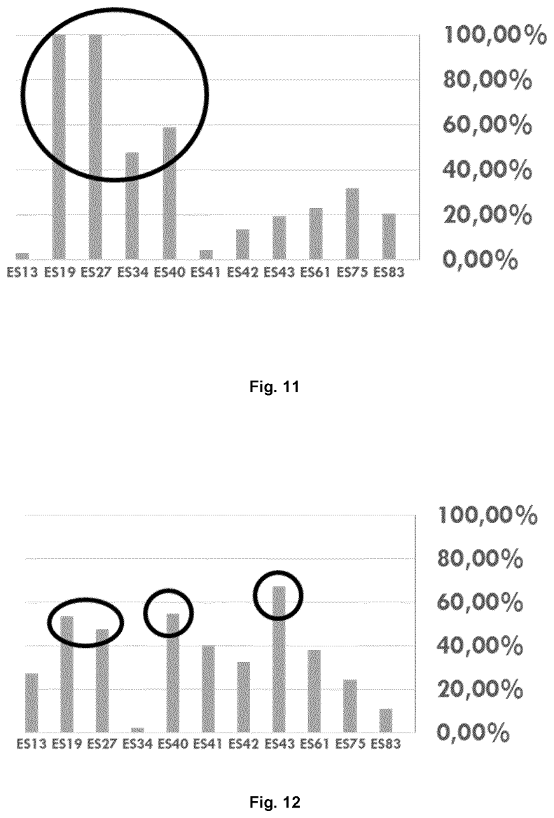

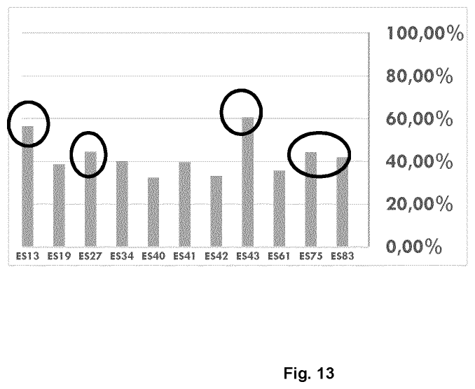

Tables 37-39 and FIGS. 11-13 show a global assessment of primary estrogen (estrone, estriol and 17β-estradiol) conjugation rate, wherein conjugated estrogens: total estrogens-free estrogens and % or rate of metabolism: conjugated estrogens/total estrogens. Those strains with higher estrogen conjugation rates as a whole are highlighted.

Table 40 shows a determination by HPLC/MS of primary estrogen and their derivatives (estrone, estriol, 17β-Estradiol, 2-hidroxi-estrone-3-metil-eter, 2-metoxi-estradiol, 4-metoxi-17β-estradiol, 4-metoxi-estrone, 16α-hydroxy-estrone, 2-hidroxi-estrone, 4-hidroxi-estrone, 17-epi-estriol, 2-metoxi-estrone) wherein the white color means qualitatively not detected; below the limit of detection [LOD] and the dark grey is when it has been qualitatively detected; above the limit of detection [LOD]).

Table 41. Concentration (mg/L) of free and conjugated estrogens present in the cell-free supernatants (SLCs) after 48 h of incubation in the presence of estrone and estradiol conjugated substrates E1G (E1G and E1) and E2G (E2G and E2) (n=3).

TABLE 41

| Strain | E1G | E1 | E2G | E2 |
|---|---|---|---|---|
| ES27 | 11.05 ± 1.68 | 1.62 ± 0.08 | nd | nd |
| | nd | nd | 0.90 ± 0.18 | 17.35 ± 3.15 |
| ES43 | 9.25 ± 1.53 | 1.04 ± 0.01 | nd | nd |
| | nd | 1.84 ± 0.03 | 0.66 ± 0.40 | 16.09 ± 1.74 |

E1: estrone, E1G: estrone-3 (β-glucuronide), E2: estradiol, E2G: estradiol-3-(β-D-glucuronide).

Table 41 shows the concentration (mg/L) of free and conjugated estrogens present in the SLC after 48 h of incubation of the selected strains in the presence of E1G (E1G and E1) and E2G (E2G and E2) (n=3).

Table 42. Percentage of free and conjugated estrogens present in the SLCs after 48 h of incubation in the presence of E1G (E1G and E1) and E2G (E2G and E2) (n=3). The percentages have been calculated taking into account that the initial concentration (Co) of E1G and E2G is 50 mg/L.

TABLE 42

| Strain | E1G | E1 | E2G | E2 |
|---|---|---|---|---|
| ES27 | 22 | 3 | nd | nd |
| | nd | nd | 2 | 35 |
| ES43 | 18 | 2 | nd | nd |
| | nd | 4 | 1 | 32 |

Table 42 shows the percentage of each of the estrogens quantified with respect to the initial concentration added. When E1G (50 mg/L) was added to the culture (results in E1G and E1 cells), part of it was deconjugated by both strains, as shown by the detection of E1 in both cultures. Most of the E1G is consumed as can be understood by the 22% and 18% of the initial amount quantified in the medium in the case of strains ES27 and ES43, respectively. No biotransformation of E1G into E2G or E2 was detected. It cannot be ruled out that part of the added E1G has been transformed into other intermediates that have not been quantified in this assay.

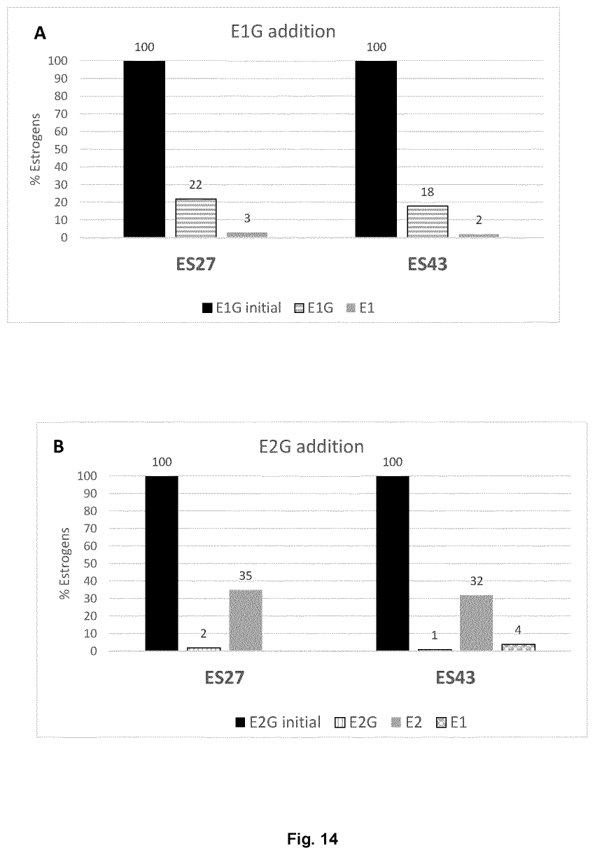

When E2G (50 mg/L) was added to the culture (results in E2G and E2 cells), only a small proportion of this compound was detected in the SLCs after 48 h of incubation with both strains, suggesting, as in the previous case, that either this compound has been consumed or metabolized to other intermediates not detected in this assay. In this case, both strains showed a very significant capacity to deconjugate estradiol, in fact very significant amounts of estradiol were detected in the SLCs. In addition, the ES43 strain was able to oxidize part of the E2G to estrone. A graphic representaion of these results are shown in FIGS. 14 (A) and (B).

The loss of E1G is accompanied by the formation of its primary degradation product E1, with the % of E1G converted to E1 being approximately 2-3%. It appears that estrone deconjugation metabolism is similar in both strains. Approximately 20% of the initially added E1G is not consumed during the first 48 h of incubation. As previously mentioned, it cannot be ruled out that other secondary metabolites not quantified in this assay may have been formed. (FIG. 14 (A)).

The loss of E2G is accompanied by the formation of the primary degradation product E2 in both cases, with the % of E2G converted to E2 being approximately 35% in both strains. In addition, the ES43 strain was able to oxidize part of this E2 to E1, a common mechanism that has been described as the preferred way to metabolize estradiol. Although this compound was not detected in the case of strain ES27, the low residual % of E2G detected after incubation with both strains could suggest that both are not only capable of deconjugating E2G but that they can metabolize this compound presumably to E1 and this compound in turn suffer further degradation (FIG. 14 (B)). FIGS. 14 (A) and (B) show that both strains (ES27 and ES 43) are capable of deconjugating both estrogen derivatives and especially E2G. Part of the parental estrogen released and in its free form could be further degraded via transformation to E1, as shown by the low proportion of estrogen detected in the cultures. It cannot be ruled out that in the case of the free form of E2, whose concentration was higher in the cultures, more time is needed for its degradation, since it first requires the transformation to its oxidized derivative, E1, as it is shown in the FIG. 14 (B).

Global results assesment shows that ES 27 and ES 43 strains are among the strains studied with higher estrogen conjugation rates and with the higher estrogen metabolization rates for the primary estrogens (estrone, estriol and 17β-estradiol). Among the estrogen derivatives analized herein, 2-metoxi-estradiol, 4-metoxi-estrone and 2-hidroxi-estrone are below the limit of detection (LOD), qualitatively not detected and 2-hidroxi-estrona-3-metil-eter, 4-metoxi-17-beta-estradiol, 16α-hidroxi-estrone and 4-hidroxi-estrone are qualitatively detected in low proportions for both strains, ES 27 and ES 43. Further, it is demostrated that both strains are able to deconjugate both estrogen derivatives studied (E1G and E2G) and especially E2G. Part of the parental estrogen released and in its free form could be further degraded via transformation to E1, as shown by the low proportion of estrone detected in the cultures. It cannot be ruled out that in the case of the free form of E2, whose concentration was higher in the cultures, more time is needed for its degradation, since it first requires the transformation to its oxidized derivative, E1.

All the tested L. salivarius strains could be considered as good candidates for metabolizing estrone and estriol. Although slight strain-associated differences were observed, all of them were able to metabolize more than 90% and 50% of the initial concentrations of estrone and estriol, respectively. It must be highlighted that only three out of the six L. salivarius strains were able to metabolize 17β-estradiol, being strains ES27 and ES43 those exhibiting the highest metabolization rates.

Microorganisms can degrade or biotransform steroid hormones by different mechanisms, being hydrolases and dioxigenases the most important enzymes that regulate these biotransformations. These mechanisms have been widely studied in microorganisms isolated from substrates (soil, sewage water) where the concentration of environmental estrogens may be relatively high. Contrary to our results, most of the bacteria-degrading estrogens that have been isolated from soils, sewage and water are especially active towards 17β-estradiol, the most abundant estrogen in environmental samples. This fact suggests that these microorganisms have developed more effective pathways for degrading or transforming this specific estrogen. In contrast, our human origin strains seem to prime conjugation of 17β-estradiol instead of degradation of this estrogen. Conjugated estrogens can be excreted through the urine or can act as reservoirs since they may be reuptaken trough enterohepatic circulation.

From a clinical perspective a wide range of hormonal dependant diseases can be positively influence due to the additional use of lactobacillus modulating oestrogens independent if together or not with classical anti estrogenic treatments. These can be benign or even malign diseases like for example the endometriosis or breast cancer.

Endometriosis is an estrogen-dependent disease which ethiology is largely unknown. It is characterized by the presence of endometrial tissue outside of the uterine cavity. The endometriosis lesions contain ERs and, therefore, the presence of circulating estrogens stimulates their growth. There are two reasons explaining why estrogen levels, and especially those of 17β-estradiol, are increased in this condition. On the one hand, the enzyme aromatase that catalyzes the conversion of androgens to estrogens is abnormally expressed in endometriotic stromal cells. On the other hand, endometriotic tissue is deficient in 17 β-hydroxysteroid dehydrogenase type 2, an enzyme that catalyses the conversion of 17β-estradiol into estrone. The fact that the L. salivarius strains tested in this study are especially prone to conjugate estradiol could help preventing the above mentioned above-estrogen-related gynaecological pathologies.

A wide range of compounds including hormones, environmental pollutants, neurotransmitters, and drugs are conjugated in the liver or gut via UDP-glucuronosyltransferase (UGT) enzymes. The capacity of L. salivarius to transfer glucuronic groups to estrogens has not been described yet. This suggests other mechanisms by which these strains may conjugate estrogens and, particularly, estradiol. In general, lactic acid bacteria have a highly conserved pathway for exopolysaccharides synthesis that contains a region in which glycosyl transferases (GTs) genes are included.

These enzymes catalyse the transference of sugar residues to a lipid carrier or to a growing polysaccharide chain. In this work, the inventors used a combination of β-glucuronidase (EC 3.2.1.31) and sulfatase as enzymatic hydrolysis buffer for quantifying total and conjugated estrogens. β-glucuronidases that belong to the family of glycoside hydrolases (GH) and that cleave glucuronic acid sugar moieties from the non-reducing end of glycosides despite their low substrate specificity toward glycosides (Lv et al. 2018 Journal of Biological Chemistry, 293(2), 433-443). We cannot rule out if our strains are effectively transferring glucuronic acid or other sugar residues to parental estrogens. In its conjugated form, estradiol is biologically inactive and unable to interact with ERS, suggesting these L. salivarius strains modulate the effect of this estrogen by blocking its action until required, probably through balancing conjugation-deconjugation mechanisms.

Nowadays, it is generally accepted that bacterial glucuronidases are a part of the physiological estrobolome; however, presence of this enzymatic activity was considered as a negative trait for selection of probiotic bacteria in the past since it was thought that the overabundance of bacteria exhibiting such activity could contribute to exacerbate estrogen-dependent pathological processes. Nevertheless, this traditional view was somehow biased since many studies describing this activity were performed in pathobionts. However, β-glucuronidases are ubiquitous enzymes that have been found in a variety of microorganisms, including lactic acid bacteria with a qualified presumption of safety and/or a generally recognized as safe status. Recently, it has been demonstrated that the administration of fecal samples rich in these enzymatic activities to mice did not correlate with an increase in tumorogenesis, suggesting the function of these enzymes could be a way of reactivating estrogens that can be further metabolized in different ways (e.g. hydrolyzed, epimerized, oxidized, methylated) in distal sites.

### BIBLIOGRAPHY

1. Kullen, M. J.; Sanozky-Dawes, R. B.; Crowell, D. C.; Klaenhammer, T. R. Use of DNA sequence of variable regions of the 16SrRNA gene for rapid and accurate identification of bacteria in the Lactobacillus acidophilus complex. J. Appl. Microbiol. 2000, 89, 511-518.
2. Bover-Cid, S.; Holzapfel, W. H. Improved screening procedure for biogenic amine production by lactic acid bacteria. Int. J. Food Microbiol. 1999, 53, 33-41.
3. 33. Lucas P, Lonvaud-funel A: Purification and partial gene sequence of the tyrosine decarboxylase of Lactobacillus brevis IOEB 9809. FEMS Microbiol Lett 2002, 211:85-89.
4. 34. Le Jeune C, Lonvaud-Funel A, ten Brink B, Hofstra H, van der Vossen JMBM: Development of a detection system for histidine decarboxylating lactic acid bacteria based on DNA probes, PCR and activity test. J Appl Bacteriol 1995, 78:316-326.
5. 35. Ladero V, Fernández M, Calles-Enríquez M, Sánchez-Llana E, Cañedo E, Martin M C, Alvarez M A: Is the production of the biogenic amines tyramine and putrescine a species-level trait in enterococci? Food Microbiol 2012, 30:132-138.
6. 36. García-Moruno E, Carrascosa A V, Muñoz R: A rapid and inexpensive method for the determination of biogenic amines from bacterial cultures by thin-layer chromatography. J Food Protect 2005, 68:625-629.
7. Zhou, J. S.; Gopal, P. K.; Hill, H. S. Potential probiotic lactic acid bacteria Lactobacillus rhamnosus (HN001), Lactobacillus acidophilus (HN017) and Bifidobacterium lactis (HN019) do not degrade gastric mucin in vitro. Int. J. Food Microbiol. 2001, 63, 81-90.
8. EFSA. Guidance on the characterisation of microorganisms used as feed additives or as production organisms. EFSA J. 2018, 16, e05206.
9. Marteau, P.; Minekus, M.; Havenaar, R.; Huis In′t Veld, J. H. J. Survival of lactic acid bacteria in a dynamic model of the stomach and small intestine: Validation and the effects of bile. J. Dairy Sci. 1997, 80, 1031-1037.
10. Martín, R.; Olivares, M.; Marín, M. L.; Fernández, L.; Xaus, J.; Rodríguez, J. M. Probiotic potential of 3 Lactobacilli strains isolated from breast milk. J. Hum. Lact. 2005, 21, 8-17.
11. Conway, P. L.; Gorbach, S. L.; Goldin, B. R. Survival of lactic acid bacteria in the human stomach and adhesion to intestinal cells. J. Dairy Sci. 1987, 70, 1-12.
12. Coconnier, M. H.; Klaenhammer, T. R.; Kernéis, S.; Bernet, M. F.; Servin, A. L. Protein-mediated adhesion of Lactobacillus acidophilus BG2FO4 on human enterocyte and mucus-secreting cell lines in culture. Appl. Environ. Microbiol. 1992, 58, 2034-2039.
13. Cohen, P. S.; Laux, D. C. Bacterial adhesion to and penetration of intestinal mucus in vitro. Methods Enzymol. 1995, 253, 309-314.
14. Juarez del Valle, M.; Laiño, J.; Savoy de Giori, G.; LeBlanc, J. G. Riboflavin producing lactic acid bacteria as a biotechnological strategy to obtain bio-enriched soymilk. Food Res. Int. 2014, 62, 1015-1019.
15. Laiño, J. E.; LeBlanc, J. G.; Savoy de Giori, G. Production of natural folates by lactic acid bacteria starter cultures isolated from artisanal Argentinean yogurts. Can. J. Microbiol. 2012, 58, 581-588.
16. Horwitz, W. Official Methods of Analysis of AOAC International; AOAC International: Gaithersburg, MD, USA, 2000.
17. Boris, S.; Suárez, J. E.; Vázquez, F.; Barbés, C. Adherence of human vaginal lactobacilli to vaginal epithelial cells and interaction with uropathogens. Infect. Immun. 1998, 66, 1985-1989.
18. Martín V, Cárdenas N, Ocaña S, Marín M, Arroyo R, Beltrán D, et al. Rectal and vaginal eradication of Streptococcus agalactiae (GBS) in pregnant women by using Lactobacillus salivarius CECT 9145, a target-specific probiotic strain. Nutrients. (2019) 11: E810.
19. Younes, J. A.; van der Mei, H. C.; van den Heuvel, E.; Busscher, H. J.; Reid, G. Adhesion forces and coaggregation between vaginal staphylococci and lactobacilli. PLOS ONE 2012, 7, e36917.
20. Padmavathi, T.; Bhargavi, R.; Priyanka, P. R.; Niranjan, N. R.; Pavitra, P. V. Screening of potential probiotic lactic acid bacteria and production of amylase and its partial purification. J. Genet. Eng. Biotechnol. 2018, 16, 357-362.
21. Narita, J.; Okano, K.; Kitao, T.; Ishida, S.; Sewaki, T.; Sung, M.-H.; Fukuda, H.; Kondo, A. Display of α-Amylase on the Surface of Lactobacillus casei Cells by Use of the PgsA Anchor Protein, and Production of Lactic Acid from Starch. Appl. Environ. Microbiol. 2006, 72, 269-275.
22. Martín R, Delgado S, Maldonado A, Jiménez E, Olivares M, Fernández L, Sobrino O J, Rodríguez J M. Isolation of lactobacilli from sow milk and evaluation of their probiotic potential. J Dairy Res. 2009 November; 76(4):418-25.
23. Brinton C C, Fusco D C. Vaccine composition for immunization against urinary tract infection caused by E. coli. U.S. Pat. No. 4,725,435, Feb. 16 1988.
24. Rafsanjany N, Lechtenberg M, Petereit F, Hensel A. Antiadhesion as a functional concept for protection against uropathogenic Escherichia coli: in vitro studies with traditionally used plants with antiadhesive activity against uropathognic Escherichia coli. J Ethnopharmacol. 2013 Jan. 30; 145(2):591-7.
25. Hilbert D W, Pascal K E, Libby E K, Mordechai E, Adelson M E, Trama J P. Uropathogenic Escherichia coli dominantly suppress the innate immune response of bladder epithelial cells by a lipopolysaccharide- and Toll-like receptor 4-independent pathway. Microbes Infect. 2008 February; 10(2):114-21.
26. de Llano D G, Esteban-Fernández A, Sánchez-Patán F, Martinlvarez P J, Moreno-Arribas M V, Bartolome B. Anti-Adhesive Activity of Cranberry Phenolic Compounds and Their Microbial-Derived Metabolites against Uropathogenic Escherichia coli in Bladder Epithelial Cell Cultures. Int J Mol Sci. 2015 May 27; 16(6):12119-30.
27. Li M, Yang D, Mei L, Yuan L, Xie A, Yuan J. Screening and characterization of purine nucleoside degrading lactic acid bacteria isolated from Chinese sauerkraut and evaluation of the serum uric acid lowering effect in hyperuricemic rats. PLOS One. 2014 Sep. 3; 9(9):e105577.
28. Nugent, R. P.; Krohn, M. A.; Hillier, S. L. Reliability of diagnosing bacterial vaginosis is improved by a standardized method of gram stain interpretation. J. Clin. Microbiol. 1991, 29, 297-301.
29. Mediano, P.; Fernández, L.; Jiménez, E.; Arroyo, R.; Espinosa-Martos, I.; Rodríguez, J. M.; Marin, M. Microbial Diversity in Milk of Women With Mastitis: Potential Role of Coagulase-Negative Staphylococci, Viridans Group Streptococci, and Corynebacteria. J. Hum. Lact. 2017, 33, 309-318.
30. Lackey, K. A.; Williams, J. E.; Meehan, C. L.; Zachek, J. A.; Benda, E. D.; Price, W. J.; Foster, J. A.; Sellen, D. W.; Kamau-Mbuthia, E. W.; Kamundia, E. W.; et al. What's Normal? Microbiomes in Human Milk and Infant Feces Are Related to Each Other but Vary Geographically: The INSPIRE Study. Front. Nutr. 2019, 6, 45.
31. Harrow, S. A.; Ravindran, V.; Butler, R. C.; Marshall, J. W.; Tannock, G. W. Real-Time Quantitative PCR Measurement of lleal Lactobacillus salivarius Populations from Broiler Chickens To Determine the Influence of Farming Practices. Appl. Environ. Microbiol. 2007, 73, 7123-7127.
32. Salvetti, E.; Harris, H. M. B.; Felis, G. E.; O'Toole, P. W. Comparative Genomics of the Genus Lactobacillus Reveals Robust Phylogroups That Provide the Basis for Reclassification. Appl. Environ. Microbiol. 2018, 84, 00993-18.
33. Ruiz-Barba, J. L.; Maldonado, A.; Jimenez-Díaz, R. Small-scale total DNA extraction from bacteria and yeast for PCR applications. Anal. Biochem. 2005, 347, 333-335.
34. Stewart, C. J.; Mansbach, J. M.; Wong, M. C.; Ajami, N.J.; Petrosino, J. F.; Camargo, C. A.; Hasegawa, K. Associations of Nasopharyngeal Metabolome and Microbiome with Severity among Infants with Bronchiolitis. A Multiomic Analysis. Am. J. Respir. Crit. Care Med. 2017, 196, 882-891.
35. Mahoney, F. L.; Barthel, D. W. Functional evaluation: The Barthel Index. Md. State Med. J. 1965, 14, 61-65.
36. Auer, S.; Reisberg, B. The GDS/FAST Staging System. Int. Psychogeriatr. 1997, 9, 167-171.
37. Bauer, J. M.; Kaiser, M. J.; Anthony, P.; Guigoz, Y.; Sieber, C. C. The Mini Nutritional Assessment-Its history, today's practice, and future perspectives. Nutr. Clin. Pract. 2008, 23, 388-396.
38. Aparicio, M.; Alba, C.; CAM Public Health Area 6 PSGO; Rodríguez, J. M.; Fernández, L. Microbiological and immunological markers in milk and infant feces for common gastrointestinal disorders: A pilot study. Nutrients 2020, 12, 634.
39. Codoner-Franch P, Tavarez-Alonso S, Murria-Estal R, Megías-Vericat J, Tortajada-Girbés M, Alonso-Iglesias E. Nitric oxide production is increased in severely obese children and related to markers of oxidative stress and inflammation. Atherosclerosis. 2011 April; 215(2):475-80.
40. Dabek, M., McCrae, S. I., Stevens, V. J., Duncan, S. H., & Louis, P. (2008). Distribution of β-glucosidase and β-glucuronidase activity and of β-glucuronidase gene gus in human colonic bacteria. FEMS microbiology ecology, 66(3), 487-495.
41. EFSA Panel on Additives and Products or Substances used in Animal Feed (FEEDAP), Rychen, G., Aquilina, G., Azimonti, G., Bampidis, V., Bastos, M. D. L., . . . & Galobart, J. (2018). Guidance on the characterisation of microorganisms used as feed additives or as production organisms. EFSA Journal, 16(3), e05206.
42. Xu, X., Roman, J. M., Issaq, H. J., Keefer, L. K., Veenstra, T. D., & Ziegler, R. G. (2007). Quantitative measurement of endogenous estrogens and estrogen metabolites in human serum by liquid Chromatography-Tandem mass spectrometry. Analytical chemistry, 79(20), 7813-7821.

What is claimed is:

1. An isolated strain of Ligilactobacillus salivarius capable of metabolizing circulating estrogens wherein the strain is the strain deposited at the “Colección Española de Cultivos Tipo (CECT)” under the accession number CECT 30632 or a variant thereof or the isolated strain of Ligilactobacillus salivarius deposited at the “Colección Española de Cultivos Tipo (CECT)” under the accession number CECT 30641 or a variant thereof.

2. The strain according to claim 1 wherein the estrogens are selected from the group consisting of estrone, estriol, 17 β-estradiol and any combination thereof.

3. A bacterial culture comprising at least one of the strains according to claim 1.

4. (canceled)

5. (canceled)

6. A pharmaceutical composition comprising a therapeutic effective dose of the isolated strain according to claim 1 and an acceptable adjuvant.

7. (canceled)

8. (canceled)

9. The method of claim 47 wherein the disease associated with high levels of estrogen is selected from a group consisting of: endometriosis, estrogen-dependent cancer, infertility, gynecomastia, erectile dysfunction, fibrocystic breast disease, hypoactive sexual desire disorder, menorrhagia, premenstrual syndrome, obesity, menopause associated hair loss and sleeping disorder.

10-22. (canceled)

23. The method according to claim 47 wherein the isolated strain comprises from about 8 log 10 CFU to about 10 log 10 CFU.

24. The method according to claim 23, wherein the isolated strain is administrated at least once a day and comprises around 9 log 10 CFU.

25. The method according to claim 47, wherein the isolated strain is administrated as a freeze-dried powder for at least 6 months or until pregnancy is achieved.

26. The method according to claim 25 wherein once pregnancy is achieved the administration of the isolated strain is continued until at least week 15 of pregnancy.

27. (canceled)

28. A food product comprising the isolated strain according to claim 1 and an appropriate amount of an edible ingredient.

29. (canceled)

30. A nutraceutical comprising the isolated strain according to claim 1.

31. A non-therapeutic method a) for the regulation of the epithelial mucosa pH in a subject, b) for the regulation of the microbiota cervicovaginal in a subject, c) for promoting an increase in the content of vaginal Lactobacillus species in a subject, d) for promoting an increase in soluble immune factors in the internal mucosal of a subject, e) for improving the mental well-being of a subject, f) for regulating the circulating estrogen levels in a subject, g) for increasing the levels of circulating riboflavin in a subject, h) for reducing the levels of circulating uric acid in a subject, i) for increasing the performance of activities of daily living in a subject, i) for increasing the nutritional state of a subject, k) for regulating the circulating levels of total cholesterol of a subject or l) for regulating the circulating levels of triglycerides of a subject which comprises the administration of the isolated strain according to claim 1 to the subject.

32-33. (canceled)

34. The non-therapeutic method according to claim 31 wherein the species of Lactobacillus increased are selected from a group consisting of: L. iners, L. crispatus, L. gasseri, L. jensenii, L. fermentum, L. salivarius and any combination thereof.

35. (canceled)

36. The non-therapeutic method according to claim 31 wherein the soluble immune factors are selected from a group consisting of TGF β1, TGF β2, VEGF and a combination thereof.

37-44. (canceled)

45. A medical device comprising an effective amount of the isolated strain according to claim 1 as an active ingredient.

46. A cosmetic composition having an effect of reducing vaginal discharge comprising the isolated strain according to claim 1 or the bacterial culture according to claim 3.

47. A method for a) the prevention and/or treatment of a disease associated with high levels of estrogen, b) the prevention and/or treatment of an infectious disease or an inflammatory disease, c) the prevention and/or treatment of a disease or syndrome selected from hyperuricemia, high cholesterol, high triglycerides and oxidative stress, and/or d) the prevention and/or treatment of primary or secondary infertility which comprises administering the isolated strain according to claim 1 to a subject in need thereof.

48. A method for increasing the rate of reproductive success in a patient who has suffered from recurrent pregnancy loss or for the prevention of miscarriage in the first trimester of pregnancy which comprises administering the isolated strain according to claim 1 to a subject in need thereof.

49. A medical device comprising an effective amount of the bacterial culture of claim 3 as an active ingredient.
